(19) United States
(12) Patent Application Publication
(10) Pub. No.: US 2024/0016956 A1
(43) Pub. Date: Jan. 18, 2024

(54) METHODS AND COMPOSITIONS FOR RESTRICTING BIODISTRIBUTION OF AAV

(71) Applicants: AFFINIA THERAPEUTICS INC., Waltham, MA (US); MASSACHUSETTS EYE AND EAR INFIRMARY, Boston, MA (US); THE SCHEPENS EYE RESEARCH INSTITUTE, INC., Boston, MA (US)

(72) Inventors: Roberto CALCEDO DEL HOYO, Waltham, MA (US); Luc H. VANDENBERGHE, Boston, MA (US)

(21) Appl. No.: 18/036,135

(22) PCT Filed: Nov. 9, 2021
(86) PCT No.: PCT/US2021/058572

§ 371 Date: May 9, 2023

Related U.S. Application Data
(60) Provisional application No. 63/256,437, filed on Oct. 15, 2021.
(60) Provisional application No. 63/111,496, filed on Nov. 9, 2020.

(51) Int. Cl. A61K 48/00; C12N 15/86; A61K 39/42; C07K 16/08; A61P 39/00

(52) U.S. Cl. CPC A61K 48/0083; C12N 15/86; A61K 39/42; C07K 16/081; A61P 39/00; C12N 2750/14143; C12N 2750/14171; A61K 2039/505

(57) ABSTRACT

The present disclosure relates to an improved method of treating a patient with a recombinant adeno-associated virus (rAAV) by adjunctively administering to the patient via systemic administration a composition comprising at least one antibody or antigen-binding fragments thereof that is capable of neutralizing the non-systemically administered rAAV. The present disclosure further provides kits for the combination therapy.

### 1. CROSS-REFERENCE TO RELATED APPLICATIONS

This application claims the benefit of U.S. Provisional Application Ser. Nos.: 63/111,496, filed Nov. 9, 2020 and 63/256,437, filed Oct. 15, 2021, the disclosures of which are hereby incorporated in their entireties by reference.

### 2. SEQUENCE LISTING

The instant application contains a Sequence Listing, which has been submitted via EFS-Web and is hereby incorporated herein by reference in its entirety. Said ASCII copy, created on Month XX, 2021, is named 47299WO_CRF_sequencelisting.txt, and is XXX bytes in size.

### 3. BACKGROUND

Adeno-associated virus (AAV) has become the vector system of choice for in vivo gene therapy. A growing variety of recombinant AAVs (rAAVs) engineered to deliver therapeutic nucleic acids have been developed and tested in in nonhuman primates and humans, and the FDA has recently approved two rAAV gene therapy products for commercialization.

Although AAV vectors are safer and less inflammatory than other viruses, toxicities have occurred following administration of high doses of rAAVs for gene therapy. Acute development of thrombocytopenia and transaminitis and liver damage have been observed in nonhuman primate studies, and liver dysfunction, acute elevation of liver enzymes, and reductions in platelets have been observed in human clinical studies. Recently, several deaths were reported in an rAAV gene therapy clinical trial for treatment of a genetic neuromuscular disorder.

While local administration of rAAVs to a target tissue or organ has been used to improve targeting and reduce systemic toxicity, complete localization is not possible, and several serotypes of AAV exhibited transduction in tissues distal from the injection site, risking systemic toxicity.

Accordingly, there is a need for methods of restricting AAV biodistribution to target tissue and organs following local or regional administration, while reducing or eliminating systemic circulation and transduction of non-target tissues and organs, such as the liver.

### 4. SUMMARY

The present disclosure relates to an improved method of treating a patient with an rAAV by adjunctively administering to the patient via systemic administration a composition comprising antibodies or antigen-binding fragments thereof that are capable of neutralizing the non-systemically administered rAAV.

Adjunctively administered neutralizing antibodies can restrict biodistribution of rAAVs to target tissues and organs by neutralizing AAVs in non-target organs and tissues. This adjunctive, systemic administration of neutralizing antibodies reduces: non-target transduction of rAAVs administered non-systemically that escape the target organs and tissues; systemic toxicity, and systemic immune responses that travel to the target tissue and organs transduced by rAAVs.

Aspects of the present disclosure include an improved method of treating a patient by non-systemic administration of a recombinant adeno-associated virus (rAAV). The improvement comprises adjunctively administering to the patient via systemic administration a composition comprising at least one antibody or antigen-binding fragment thereof that is capable of neutralizing the non-systemically administered rAAV.

In some embodiments, the at least one antibody or antigen-binding fragment binds to at least one capsid protein of the rAAV. In some embodiments, the rAAV has a capsid protein of an AAV selected from: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

In some embodiments, the at least one antibody is an IgG antibody.

In some embodiments, the composition is capable of neutralizing an rAAV selected from AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

In some embodiments, the composition comprises a monoclonal antibody.

In some embodiments, the composition comprises polyclonal antibodies comprising a mixture of monoclonal antibodies.

In some embodiments, the composition comprises polyclonal antibodies obtained from at least 100 donors. In some embodiments, the polyclonal antibodies obtained from at least 500, at least 1000, at least 5000, or at least 10,000 donors.

In some embodiments, the composition is intravenous immunoglobulin (IVIG). In some embodiments, the IVIG is a pool of antibodies obtained from: Gammagard™ Liquid® (Baxter HealthCare Corp), Gammagard™ S/D, Gammaplex™, Bivigam™, Carimune™ NF, Gamunex-C, Gammaked™, Flebogamma™ DIF, Octagam™, and Privigen™.

In some embodiments, the composition comprises recombinant polyclonal antibodies. In some embodiments, the composition comprises recombinant IVIG (rIVIG).

In some embodiments, the at least one antibody is an antigen-binding fragment selected from: a single chain FIT (scFv), a Fab, a (Fab′)₂, and an (ScFv)₂. In some embodiments, the composition comprises: a bispecific antibody, a single-domain antibody (dAb), a diabody (db), a nanobody, and a unibody.

In some embodiments, the composition is administered by intravenous injection or infusion.

In some embodiments, the composition is IVIG and the IVIG is administered at a dose ranging from 1-2000 mg/kg patient weight. In some embodiments, the IVIG is administered at a dose ranging from 1-2 mg/kg patient weight. In some embodiments, IVIG is administered at a dose ranging from 1-10 mg/ml. In some embodiments, the IVIG is administered at a dose ranging from 1-4 mg/ml. In some embodiments, IVIG is administered as a single-dose. In some embodiments, the IVIG is administered daily.

In some embodiments, the IVIG is administered 2 times a day, three times a day, or four times a day.

In some embodiments, the IVIG is administered 2 times a day, three times a day, or four times a day. In some embodiments, the IVIG is administered for no more than one week.

In some embodiments, the IVIG is administered for at least 5 minutes, at least 10 minutes, at least 15 minutes, at least 20 minutes, at least 25 minutes, or at least 30 minutes.

In some embodiments, the composition is administered before administration of the rAAV to the patient. In some embodiments, the composition is administered at least 30 minutes before, at least 1 hour before, at least 2 hours before, at least 4 hours before at least 6 hours before, at least 12 hours, or at least 24 hours before, administration of the rAAV to the patient. In some embodiments, the composition is administered 30 minutes before, 1 hour before, 2 hours before, 4 hours before, 6 hours before, 12 hours before, or 24 hours before, administration of the rAAV to the patient. In some embodiments, the composition is administered no more than one week before administration of the rAAV to the patient.

In some embodiments, the composition is administered after administration of the rAAV to the patient. In some embodiments, the composition is administered no more than 30 minutes after, at least 1 hour after, at least 2 hours after, at least 4 hours after, at least 6 hours after, at least 24 hours after, or no more than one week after administration of the rAAV to the patient.

In some embodiments, the composition is administered on the same day as administration of the rAAV to the patient.

In some embodiments, the composition is administered at a sufficient amount to alter biodistribution of the rAAV following administration to the patient.

In some embodiments, altering rAAV biodistribution reduces infection of liver cells. In some embodiments, altering rAAV biodistribution reduces infection of cells outside of the target site.

In some embodiments, the composition is administered at a sufficient amount to reduce liver toxicity of the rAAV following administration to the patient. In some embodiments, the composition is administered at a sufficient amount to reduce toxicity of the rAAV outside of the target site following administration to the patient.

In some embodiments, the composition is administered at a sufficient amount to neutralize rAAV released from the target site.

In some embodiments, the target site is selected from: brain, spinal cord, muscle, eye, and ear of the patient. In some embodiments, the target site is not within the central nervous system of the patient.

Aspects of the present disclosure include a method of treating a patient comprising the steps of: non-systemically administering to a target site of the patient a first pharmaceutical composition comprising a recombinant adeno associated virus (rAAV); and adjunctively administering to the patient via systemic administration a second pharmaceutical composition comprising at least one antibody that is capable of neutralizing the non-systemically administered rAAV.

In some embodiments, the at least one antibody binds to a capsid protein of the rAAV. In some embodiments, the rAAV has a capsid protein of an AAV selected from: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI. In some embodiments, the AAV is AAV8 or AAV9. In some embodiments, the AAV is rh.10. In some embodiments, the AAV is hu.68.

In some embodiments, the antibody is an IgG antibody.

In some embodiments, the second pharmaceutical composition is capable of neutralizing an AAV selected from AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

In some embodiments, the at least one antibody is a monoclonal antibody. In some embodiments, the second pharmaceutical composition comprises polyclonal antibodies comprising a mixture of monoclonal antibodies.

In some embodiments, the second pharmaceutical composition comprises polyclonal antibodies obtained from at least 100 donors. In some embodiments, the second pharmaceutical composition comprises polyclonal antibodies obtained from at least 500, 1000, 5000, or 10,000 donors.

In some embodiments, the second pharmaceutical composition comprises intravenous immunoglobulin (IVIG). In some embodiments, the IVIG is a pool of antibodies obtained from: Gammagard™ Liquid® (Baxter HealthCare Corp), Gammagard™ S/D, Gammaplex™, Bivigam™, Carimune™ NF, Gamunex-C, Gammaked™, Flebogamma™ DIF, Octagam™, and Privigen™.

In some embodiments, the second pharmaceutical composition comprises recombinant polyclonal antibodies.

In some embodiments, the second pharmaceutical composition comprises recombinant IVIG (rIVIG). In some embodiments, the at least one antibody comprises an antigen-binding fragment selected from: a single chain FIT (scFv), a Fab, a (Fab′)₂, and an (ScFv)₂.

In some embodiments, the at least one antibody comprises: a bispecific antibody, a single-domain antibody (dAb), a diabody (db), a nanobody, a unibody, and a diabody.

In some embodiments, the first pharmaceutical composition is administered locally.

In some embodiments, the target site is within the central nervous system. In some embodiments, the target site is selected from: brain, spinal cord, muscle, eye, and ear of the patient.

In some embodiments, the first pharmaceutical composition is administered by: intrathecal injection or infusion, intraocular injection, intravitreal injection or infusion, inner ear injection or infusion, intracerebroventricular injection or infusion, intracisternal magna injection (ICM) or infusion, subretinal injection or infusion, or intramuscular injection or infusion. In some embodiments, intrathecal injection or infusion is lumbar injection or infusion. In some embodiments, the first pharmaceutical composition is administered to the brain or spinal cord by intracerebral, intrathecal, intracranial, intracerebroventricular, or cisterna magna administration.

In some embodiments, the second pharmaceutical composition is administered systemically. In some embodiments, the second pharmaceutical composition is administered by intravenous injection or infusion.

In some embodiments, the second pharmaceutical composition comprises IVIG and the IVIG is administered at a dose ranging from 1-2000 mg/kg.

In some embodiments, the IVIG is administered at a dose ranging from 1-2 mg/kg. In some embodiments, the IVIG is administered at a dose ranging from 1-10 mg/ml. In some embodiments, the IVIG is administered at a dose ranging from 1-4 mg/ml. In some embodiments, IVIG is administered as a single-dose.

In some embodiments, the IVIG is administered daily. In some embodiments, the IVIG is administered at least 2 times a day.

In some embodiments, the second pharmaceutical composition is administered before administration of the first pharmaceutical composition to the patient.

In some embodiments, the second pharmaceutical composition is administered at least 30 minutes before, at least 1 hour before, at least 2 at least 6 hours, at least 12 hours or at least 24 hours before administration of the first pharmaceutical composition to the patient. In some embodiments, the second pharmaceutical composition is administered 30 minutes before, 1 hour before, 2 hours, 6 hours, 12 hours or 24 hours before administration of the first pharmaceutical composition to the patient.

In some embodiments, the second pharmaceutical composition is administered at least one day before, at least one week before, or at least two weeks before administration of the first pharmaceutical composition to the patient.

In some embodiments, the second pharmaceutical composition is administered after administration of the first pharmaceutical composition to the patient.

In some embodiments, the second pharmaceutical composition is administered at least 30 minutes after, at least 1 hour after, or at least 2 hours after administration of the first pharmaceutical composition to the patient.

In some embodiments, the second pharmaceutical composition is administered at least one day after, at least two days after, or at least one week after administration of the first pharmaceutical composition to the patient.

In some embodiments, the first pharmaceutical composition and the second pharmaceutical composition are administered on the same day.

In some embodiments, the second pharmaceutical composition is administered at a sufficient amount to alter biodistribution of the rAAV following administration to the patient.

In some embodiments, altering AAV biodistribution reduces rAAV infection of liver cells. In some embodiments, altering AAV biodistribution reduces rAAV infection of cells outside of the target site.

In some embodiments, the second pharmaceutical composition is administered at a sufficient amount to reduce liver toxicity of the rAAV following administration to the patient.

In some embodiments, the second pharmaceutical composition is administered at a sufficient amount to reduce toxicity of the rAAV outside of the target site following administration to the patient.

In some embodiments, the target site is selected from: brain, spinal cord, muscle, eye, and ear of the patient.

In some embodiments, the target site is not within the central nervous system.

Aspects of the present disclosure include a kit for a combination therapy comprising: a first pharmaceutical composition comprising a recombinant adeno associated virus (rAAV); and a second pharmaceutical composition comprising at least one antibody capable of neutralizing non-systemically administered rAAV of the first pharmaceutical composition.

In some embodiments, the at least one antibody binds to a capsid protein of the rAAV. In some embodiments, the capsid protein of an AAV is selected from AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

In some embodiments, the at least one antibody comprises IgG.

In some embodiments, the second pharmaceutical composition is capable of neutralizing an AAV selected from AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

In some embodiments, the at least one antibody is a monoclonal antibody.

In some embodiments, the second pharmaceutical composition comprises polyclonal antibodies obtained from at least 100 donors. In some embodiments, the second pharmaceutical composition comprises polyclonal antibodies obtained from at least 500, 1000, 5000, or 10,000 donors.

In some embodiments, the second pharmaceutical composition comprises intravenous immunoglobulin (IVIG).

In some embodiments, the second pharmaceutical composition comprises recombinant intravenous immunoglobulin (rIVIG).

In some embodiments, the IVIG is a pool of antibodies obtained from: Gammagard™ Liquid® (Baxter HealthCare Corp), Gammagard™ S/D, Gammaplex™, Bivigam™, Carimune™ NF, Gamunex-C, Gammaked™, Flebogamma™ DIF, Octagam™, and Privigen™.

In some embodiments, the second pharmaceutical composition comprises recombinant polyclonal antibodies.

In some embodiments, the second pharmaceutical composition comprises an AAV-neutralizing antigen-binding fragment selected from: a single chain Fv (scFv), a Fab, a (Fab′)₂, and an (ScFv)₂.

In some embodiments, the second pharmaceutical composition comprises: a bispecific antibody, a single-domain antibody (dAb), a diabody (db), a nanobody, a unibody, and a diabody.

### 5. BRIEF DESCRIPTION OF THE SEVERAL VIEWS OF THE DRAWINGS

These and other features, aspects, and advantages of the present invention will become better understood with regard to the following description, and accompanying drawings where:

FIGS. 1A-1G show vector genome copy biodistribution in various tissues of mice treated with AAV9.CAG.GFP with or without administration of IVIG, as measured by ddPCR. Y-axis represents the ratio of eGFP DNA copy number over RPP30 DNA copy number and x-axis represents treatment conditions. Each graph shows eGFP expression in brain (FIG. 1A), liver (FIG. 1B), spleen (FIG. 1C), cervical DRG (FIG. 1D), lumbar DRG (FIG. 1E), heart (FIG. 1F), or kidney (FIG. 1G). Data points indicate individual animals, line indicates mean. Statistics were performed using One-Way ANOVA and Tukey's multiple comparisons test.

FIGS. 2A-2G show eGFP mRNA expression in various tissues of mice treated with AAV9.CAG.GFP with or without administration of IVIG, as measured by RT-ddPCR. Y-axis represents the ratio of eGFP transcripts over RPP30 transcripts and x-axis represents treatment conditions. Each graph shows eGFP expression in brain (FIG. 2A), liver (FIG. 2B), spleen (FIG. 2C), cervical/thoracic DRG (FIG. 2D), lumbar DRG (FIG. 2E), heart (FIG. 2F), or kidney (FIG. 2G). Data points indicate individual animals, line indicates mean. Statistics were performed using One-Way ANOVA and Tukey's multiple comparisons test.

FIGS. 3A-3G show anti-GFP immunohistochemistry data in various tissues of mice treated with AAV9.CAG.GFP with or without treatment with IVIG. Anti-GFP immunohistochemistry was performed on cross sections of brain (FIG. 3A), liver (FIG. 3B), spleen (FIG. 3C), cervical/thoracic DRG (FIG. 3D), lumbar DRG (FIG. 3E), heart (FIG. 3F), or kidney (FIG. 3G) of animals transduced with: saline; AAV9.CAG.GFP ICV; AAV9.CAG.GFP ICV with 2 hour IVIG; AAV9.CAG.GFP with 24 hour IVIG; or AAV9.CAG.GFP IV. Cells positive for GFP are stained brown (indicated as dark gray cells on FIGS. 3A-3G). DR—dorsal root. DRG—dorsal root ganglion.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

The term “rAAV” as used herein refers to a virion comprising a recombinant nucleic acid construct packaged in an AAV capsid. The recombinant construct comprises a polynucleotide payload positioned between AAV inverted terminal repeats. The payload can be an expressible polynucleotide that encodes a protein (e.g., therapeutic protein) or a miRNA, siRNA, or guide RNA for gene editing machinery such as CRISPR. The payload can be a DNA homology construct for gene-editing promoted homology directed repair.

The term “neutralizing” or “neutralization” as used herein refers to a measurable decrease in the toxicity and/or circulating level of an AAV. The term “neutralizing antibody” refers to an antibody capable of decreasing the toxicity and/or circulating level of an AAV. Neutralizing antibodies can bind to a virus in a manner that blocks infection, blocks interactions with the receptor, inhibits uncoating of the genome, aggregates the virus, induces structural alteration in the capsid, prevents viral disassembly and uncoating (a step necessary to release the DNA), etc. Some neutralizing antibodies have several of these effects.

The term “antibody” (also used interchangeably with “immunoglobulin”) encompasses polyclonal, monoclonal, and allogeneic antibody preparations where the antibody may be of any class of interest (e.g., IgM, IgG, IgA, and subclasses thereof), as well as preparations including hybrid antibodies, altered antibodies, F(ab′)₂ fragments, F(ab) molecules, FIT fragments, single chain fragment variable displayed on phage (scFv), nanobodies, single chain antibodies, single domain antibodies, chimeric antibodies, humanized antibodies, and functional fragments thereof which exhibit immunological binding properties of the parent antibody molecule. The antibodies may be conjugated to other moieties, and/or may be bound to a support (e.g., a solid support), such as a polystyrene plate or bead, test strip, and the like.

Antibodies and fragments of the present disclosure encompass those that are bispecific or multispecific. Bispecific or multispecific antibodies useful in the methods described herein may have binding specificities for at least two different epitopes, at least one of which is an epitope of a rAAV. The rAAV binding antibodies and fragments can also be heteroantibodies. Heteroantibodies are two or more antibodies, or antibody binding fragments (e.g., Fab) linked together, each antibody or fragment having a different specificity.

The phrase “binds to”, “specifically binds to” or “specifically immunoreactive with”, when referring to an antibody refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to a protein or virion under such conditions may require an antibody that is selected for its specificity for a particular protein. For example, AAV-neutralizing antibodies can be raised to AAV7 that specifically bind to AAV7 virions, and not to other virions present in tissue, organs, or bodily fluids. A variety of immunoassay formats may be used to select antibodies specifically immunoreactive with a particular protein. For example, solid-phase ELISA immunoassays are routinely used to select monoclonal antibodies specifically immunoreactive with a protein.

The term “biodistribution” as used herein refers to the spread of AAV after administration, and its localization and persistence in tissues, body fluids, or organs. For example, biodistribution of the AAV can include the amount of vector uptake and transduction to and into cells in the liver or other organs, and expression of an expressible polynucleotide, such as a polynucleotide expressing a transgene, RNA, or DNA. In some embodiments, biodistribution analysis includes, but is not limited to, quantification of the expression of viral genomes by qPCR and deep sequencing, and AAV infection.

The term “AAV capsid protein” as used herein refers to a VP1, VP2, or VP3 capsid protein of a naturally occurring AAV serotype or a non-naturally occurring VP1, VP2, or VP3 capsid protein available or known in the art.

The term “adjunctive administration” or “adjunctively administering” refers to administering one therapeutic agent in sufficient temporal proximity to the other therapeutic agent to provide an effect. Adjunctive administration includes administration of one therapeutic agent concurrent with (at the same time), sequential to (at a different time but on the same day, e.g., during the same patient visit), or separate from (on a different day) administration of the other therapeutic agent.

The term “IVIG” or “intravenous immunoglobulin” as used herein has the meaning commonly understood in the clinical art. IVIG includes a concentrated and diverse collection of antibodies against many antigens. IVIG is usually administered to a subject intravenously but can be administered in a different route.

The term “polyclonal antibodies” as used herein refers to a population of heterogeneous antibodies which are usually produced by different B cell clones. They can recognize and bind to many different epitopes of a single antigen or to many antigens. Polyclonal antibodies can be either monospecific or polyspecific. Polyclonal antibodies can be, but are not limited to, a mixture of monoclonal antibodies.

The term “recombinant polyclonal antibodies” as used herein refers to a population of heterogenous antibodies produced by a library of recombinant constructs, each of the recombinant constructs containing a coding sequence of an antibody or an antigen binding fragment. The recombinant polyclonal antibodies can recognize and bind to many different epitopes of a single antigen. In some embodiments, the recombinant polyclonal antibodies recognize and bind to many different antigens.

The term “target site” as used herein refers to a cell, tissue, organ or body part where a composition is administered.

Throughout this specification and claims, “composition” and “pharmaceutical composition” are used interchangeably.

Throughout this specification and claims, the word “comprise” or variations such as “comprises” or “comprising”, will be understood to imply the inclusion of a stated integer or group of integers but not the exclusion of any other integer or group of integers.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

It must be noted that as used herein and in the appended claims, the singular forms “a,” “an,” and “the” include plural referents unless the context clearly dictates otherwise. Thus, for example, reference to “an antibody or antigen binding fragment” includes a plurality of such antibodies and antigen binding fragments and reference to “the recombinant adeno-associated virus” includes reference to one or more recombinant adeno-associated viruses and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 31, 32, 33, 34, 35, 36, 37, 38, 39, 40, 41, 42, 43, 44, 45, 46, 47, 48, 49, and 50.

6.3. Method of Treatment

In a first aspect, the current disclosure provides an improved method of treating a patient by non-systemic administration of a recombinant adeno-associated virus (rAAV). The improvement comprises adjunctively administering to the patient via systemic administration a composition comprising at least one antibody or antigen-binding fragment thereof that is capable of neutralizing the non-systemically administered rAAV.

The antibody or antigen-binding fragments thereof are capable of neutralizing the non-systemically administered rAAV, in particular rAAV that exits the site of administration and enters a systemic compartment. A systemic compartment of the patient can include, but is not limited to, a non-target organ or non-target tissue, or a location within the patient's circulatory system that is outside of the target site.

The adjunctively administered antibody composition reduces or eliminates transduction of tissues by rAAV that escapes the site of administration and reaches the systemic circulation. In particular, the systemic administration of antibodies or antigen binding fragments reduces non-target (e.g., liver) transduction by rAAV circulating systemically in the patient, reducing or eliminating toxicity (e.g., liver toxicity). The adjunctively administered antibody composition can also reduce or eliminate induction of systemic immune responses to the rAAV.

In another aspect of the present disclosure, methods of treating a patient are provided. The methods comprise the steps of non-systemically administering to the patient a first pharmaceutical composition comprising a recombinant adeno associated virus (rAAV); and adjunctively administering to the patient via systemic administration a second pharmaceutical composition comprising at least one antibody or antigen-binding fragment thereof that is capable of neutralizing the non-systemically administered rAAV.

In some embodiments, the patient has never been treated with a therapy comprising rAAV prior to being treated with the method described herein. In some embodiments, the patient has been treated with a therapy comprising rAAV prior to being treated with the method described herein.

6.3.1. Recombinant Adeno-Associated Virus (rAAV)

In the methods described herein, a first composition comprising recombinant AAV (rAAV) is administered non-systemically.

In some embodiments, the payload of the rAAV recombinant nucleic acid construct is an expressible polynucleotide. In certain embodiments, the expressible polynucleotide encodes a protein (e.g., encoding a therapeutic protein). In certain embodiments, the expressible polynucleotide can be transcribed to provide a guide RNA, a trans-activating CRISPR RNA (tracrRNA), a messenger RNA (mRNA), a microRNA (miRNA), or a shRNA.

In some embodiments, the payload provides a DNA homology construct for homology directed repair.

In some embodiments, the rAAV is Zolgensma, Luxturna, other therapeutically developed rAAV drug, or a variation thereof,

In some embodiments, the rAAV comprises a VP1, VP2, and/or VP3 capsid protein of a naturally occurring AAV serotype. In some embodiments, the rAAV comprises a non-naturally occurring VP1, VP2, and/or VP3 capsid protein. In certain of these embodiments, the non-naturally occurring VP1, VP2, or VP3 capsid protein differs in primary amino acid sequence from naturally occurring capsids. In certain embodiments, the non-naturally occurring capsid includes a biologic or chemical alteration or variation of a naturally occurring AAV capsid protein other than or in addition to a change in the primary amino acid sequence.

In various embodiments, the capsid proteins are those of an AAV1, AAV2, AAV3B, AAV5, AAV6, AAV8, or AAV9 naturally occurring AAV serotype. In various embodiments, the capsid protein is selected from capsid proteins disclosed in PCT/US2014/060163, U.S. Pat. No. 9,695,220, PCT/US2016/044819, PCT/US2018/032166, PCT/US2019/031851, and PCT/US2019/047546, which are incorporated herein by reference in their entireties.

In some embodiments, the capsid protein is a capsid protein of AAV9 (Genbank Ace. No: AAS99264.1), AAV1 (Genbank Ace. No: AAD27757.1), AAV2 (Genbank Ace. No: AAC03780.1), AAV3 (Genbank Ace. No: AAC55049.1), AAV3b (Genbank Ace. No: AF028705.1), AAV4 (Genbank Ace. No: AAC58045.1), AAV5 (Genbank Ace. No: AAD13756.1), AAV6 (Genbank Ace. No: AF028704.1), AAV7 (Genbank Ace. No: AAN03855.1), AAV 8 (Genbank Ace. No: AAN03857.1), AAV10 (Genbank Ace. No: AAT46337.1), AAVrh10 (Genbank Ace. No: AY243015.1), AAV11 (Genbank Ace. No: AAT46339.1), AAV12 (Genbank Ace. No: ABI16639.1), AAV13 (Genbank Ace. No: ABZ10812.1), or AAVpol (Genbank Ace. No: FJ688147.1).

In various embodiments, the AAV capsid protein is a VP1, VP2 or VP3 capsid protein of AAV selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh. 35-B; rh.58-B; rh.28-B; rh. 51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu. 54-C; hu.53-C; hu.60-C; hu. 55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh. 62-D; AAV7-D; rh.52-E; rh. 51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; and hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI, or a variant of thereof.

In some embodiments, the capsid protein is a vp1 capsid protein having a sequence selected from: SEQ ID NO: 54 (AAV1 (AAD27757)), SEQ ID NO: 55 (AAV2 (AAC03780)), SEQ ID NO: 56 (AAV3 (AAC55049)), SEQ ID NO: 57 (AAV5 (AAD13756)), SEQ ID NO: 58 (AAV6 (AAB95450)), SEQ ID NO: 59 (AAV7 (AF513851_2)), SEQ ID NO: 60 (AAV8 (AF513852_2)), SEQ ID NO: 61 (AAV9 (AAS99264)), SEQ ID NO: 62 (AAV10 (AAT46337)), SEQ ID NO: 63 (AAV hu.68), SEQ ID NO: 64 (AAV LK03), SEQ ID NO: 65 (AAV hu.1 (AAS99260)), SEQ ID NO: 66 (AAV hu.2 (AAS99270)), SEQ ID NO: 67 (AAV hu.3 (AAS99280)), SEQ ID NO: 68 (AAV hu.4 (AAS99287)), SEQ ID NO: 69 (AAV hu.6 (AAS99306)), SEQ ID NO: 70 (AAV hu.7 (AAS99313)), SEQ ID NO: 71 (AAV hu.9 (AAS99314)), SEQ ID NO: 72 (AAV hu.10 (AAS99261)), SEQ ID NO: 73 (AAV hu.11 (AAS99262)), SEQ ID NO: 74 (AAV hu.15 (AAS99265)), SEQ ID NO: 75 (AAV hu.16 (AAS99266)), SEQ ID NO: 76 (AAV hu.17 (AAS99267)), SEQ ID NO: 77 (AAV hu.18 (AAS99268)), SEQ ID NO: 78 (AAV hu.20 (AAS99271)), SEQ ID NO: 79 (AAV hu.21 (AAS99272)), SEQ ID NO: 80 (AAV hu.22 (AAS99273)), SEQ ID NO: 81 (AAV hu.23 (AAS99274)), SEQ ID NO: 82 (AAV hu.25 (AAS99276)), SEQ ID NO: 83 (AAV hu.27 (AAS99277)), SEQ ID NO: 84 (AAV hu.28 (AAS99278)), SEQ ID NO: 85 (AAV hu.29 (AAS99279)), SEQ ID NO: 86 (AAV hu.31 (AAS99281)), SEQ ID NO: 87 (AAV hu.32 (AAS99282)), SEQ ID NO: 88 (AAV hu.34 (AAS99283)), SEQ ID NO: 89 (AAVhu.37 (AAS99285)), SEQ ID NO: 90 (AAV hu.39 (AAS99286)), SEQ ID NO: 91 (AAV hu.41 (AAS99289)), SEQ ID NO: 92 (AAV hu.42 (AAS99290)), SEQ ID NO: 93 (AAV hu.43 (AAS99291)), SEQ ID NO: 94 (AAV hu.44 (AAS99292)), SEQ ID NO: 95 (AAV hu.45 (AAS99293)), SEQ ID NO: 96 (AAV hu.46 (AAS99294)), SEQ ID NO: 97 (AAV hu.47 (AAS99295)), SEQ ID NO: 98 (AAV hu.48 (AAS99296)), SEQ ID NO: 99 (AAV hu.51 (AAS99298)), SEQ ID NO: 100 (AAV hu.52 (AAS99299)), SEQ ID NO: 101 (AAV hu.53 (AAS99300)), SEQ ID NO: 102 (AAV hu.54 (AAS99301)), SEQ ID NO: 103 (AAV hu.55 (AAS99302)), SEQ ID NO: 104 (AAV hu.56 (AAS99303)), SEQ ID NO: 105 (AAV hu.57 (AAS99304)), SEQ ID NO: 106 (AAV hu.60 (AAS99307)), SEQ ID NO: 107 (AAV hu.61 (AAS99308)), SEQ ID NO: 108 (AAV hu.63 (AAS99309)), SEQ ID NO: 109 (AAV hu.66 (AAS99311)), SEQ ID NO: 110 (AAV hu.67 (AAS99312)), SEQ ID NO: 111 (AAV rh.10 (AA088201)), SEQ ID NO: 112 (AAV rh.13 (AA088199)), SEQ ID NO: 113 (AAV rh.19 (AA088194)), SEQ ID NO: 114 (AAV rh.22 (AA088192)), SEQ ID NO: 115 (AAV rh.23 (AA088191)), SEQ ID NO: 116 (AAV rh.24 (AA088190)), SEQ ID NO: 117 (AAV rh.35 (AA088186)), SEQ ID NO: 118 (AAV rh.43 (AAS99245)), SEQ ID NO: 119 (AAV rh.48 (AAS99246)), SEQ ID NO: 120 (AAV rh.49 (AAS99247)), SEQ ID NO: 121 (AAV rh.50 (AAS99248)), SEQ ID NO: 122 (AAV rh.51 (AAS99249)), SEQ ID NO: 123 (AAV rh.52 (AAS99250)), SEQ ID NO: 124 (AAV rh.53 (AAS99251)), SEQ ID NO: 125 (AAV rh.54 (AAS99252)), SEQ ID NO: 126 (AAV rh.55 (AAS99253)), SEQ ID NO: 127 (AAV rh.57 (AAS99254)), SEQ ID NO: 128 (AAV rh.58 (AAS99255)), SEQ ID NO: 129 (AAV rh.62 (AAS99258)), SEQ ID NO: 130 (AAV rh.64 (AAS99259)), SEQ ID NO: 131 (AAV rh.56 (JA400164)), SEQ ID NO: 143 (Anc80L1), SEQ ID NO: 144 (Anc80L27), SEQ ID NO: 145 (Anc80L33), SEQ ID NO: 146 (Anc80L36), SEQ ID NO: 147 (Anc80L44), SEQ ID NO: 148 (Anc80L59), SEQ ID NO: 149 (Anc80L60), SEQ ID NO: 150 (Anc80L62), SEQ ID NO: 151 (Anc82DI), and SEQ ID NO: 152 (AAV rh.74).

In some embodiments, the protein is a vp1 capsid protein having a sequence selected from: SEQ ID NO: 132 (Anc80), SEQ ID NO: 133 (Anc81 (AKU89596)), SEQ ID NO: 134 (Anc82 (AKU89597)), SEQ ID NO: 135 (Anc83 (AKU89598)), SEQ ID NO: 136 (Anc84 (AKU89599)), SEQ ID NO: 137 (Anc94) SEQ ID NO: 138 (Anc110 (AKU89600)), SEQ ID NO: 139 (Anc113 (AKU89601)), SEQ ID NO: 140 (Anc126 (AKU89602)), SEQ ID NO: 141 (Anc127 (AKU89603)), and SEQ ID NO: 142 (Anc80L65 (AKU89595)).

The AAV capsid protein can be a vp2 or vp3 protein having a part of one of the vp1 sequences. For example, vp2 protein can have a sequence corresponding to amino acids 138 to 736 of AAV9 vp1 and vp3 protein can have a sequence corresponding to amino acids 138 to 736 of AAV9 vp1 protein. When a SEQ ID NO for a library sequence is used in this disclosure, it refers to a sequence of any one member of the library. In some embodiments, the AAV capsid protein is a variant of the vp1, vp2, or vp3 protein described herein.

In some embodiments, the rAAV is administered in a first composition. The first pharmaceutical composition comprises a therapeutically effective amount of rAAV and a physiologically compatible carrier. Suitable carriers include saline, artificial cerebrospinal fluid, and other carriers designed for local or regional administration.

The dose of a viral vector administered to a subject will depend primarily on factors such as the clinical condition being treated, the route of administration, and the age, weight, and health of the subject. For example, a therapeutically effective dosage of a viral vector to be administered to a human subject generally is in the range of from about 0.1 ml to about 10 ml of a solution containing the viral vector. The therapeutically effective dosage can comprise about 1×10¹ to 2×10¹⁶ genome copies (GCs) of viruses (e.g., about 1×10¹ to 1×10¹⁶; about 1×10³ to 1×10¹⁵; about 1×10³ to 1×10¹⁴ GCs, etc.). In some embodiments, 1×10⁹ to 1×10¹⁵ genome copies (GCs) of viruses are administered per patient. In some embodiments, 1×10¹⁰ to 1×10¹⁶ genome copies (GCs) of viruses are administered per patient. In some embodiments, 1×10¹² to 1×10¹⁶ genome copies (GCs) of viruses are administered per patient. In some embodiments, 1×10¹² to 1×10¹⁴ genome copies (GCs) of viruses are administered per patient. In some embodiments, 1×10¹³ to 1×10¹⁴ genome copies (GCs) of viruses are administered per patient. In some embodiments, 1×10¹³ to 1×10¹⁸ genome copies (GCs) of viruses are administered per patient. In some embodiments, 1×10¹³ to 1×10²⁰ genome copies (GCs) of viruses are administered per patient.

In certain embodiments, a therapeutically effective dosage of a viral vector to be administered to a human subject generally is in the range of from about 0.1 ml to about 10 ml of a solution containing about 1×10¹ to 2×10¹⁶ genome copies (GCs) of viruses (e.g., about 1×10¹ to 1×10¹⁶; about 1×10³ to 1×10¹⁵; about 1×10³ to 1×10¹⁴ GCs, etc.).

Transduction and/or expression of a transgene can be monitored at various time points following administration by DNA, RNA, or protein assays. In some instances, the levels of expression of the transgene can be monitored to determine the frequency and/or amount of dosage. Dosage regimens similar to or lower than those described for therapeutic purposes can be utilized for immunization.

6.3.2. AAV-Neutralizing Antibodies

The method provided in the present disclosure comprises the step of systemically administering a composition comprising at least one antibody, or an antigen-binding fragment thereof, that is capable of neutralizing the non-systemically administered rAAV.

In various embodiments, the non-systemically administered rAAV includes a capsid protein of an AAV as described in section 5.3.1. and the composition comprising the at least one antibody or antigen binding fragment that is capable of neutralizing an AAV as described in section 5.3.1.

In some embodiments, the composition comprising at least one antibody includes at least two antibodies, at least three antibodies, at least four antibodies, at least five antibodies, at least six antibodies, at least seven antibodies, at least eight antibodies, at least nine antibodies, or at least ten antibodies. In various embodiments, the composition comprises at least 10 antibodies, at least 50 antibodies, at least 100 antibodies, at least 150 antibodies, at least 200 antibodies, at least 250 antibodies, at least 300 antibodies, at least 350 antibodies, at least 400 antibodies, at least 450 antibodies, at least 500 antibodies, at least 750 antibodies, at least 1000 antibodies, at least 1500 antibodies, at least 2000 antibodies, at least 2500 antibodies, at least 3000 antibodies, at least 3500 antibodies, at least 4000 antibodies, at least 4500 antibodies, at least 5000 antibodies, at least 5500 antibodies, at least 6000 antibodies, at least 6500 antibodies, at least 7000 antibodies, at least 7500 antibodies, at least 8000 antibodies, at least 8500 antibodies, at least 9000 antibodies, at least 9500 antibodies, or at least 10,000 antibodies. In various embodiments, the composition comprises at least 500 antibodies, at least 1000 antibodies, at least 5000 antibodies, at least 10,000 antibodies, at least 15,000 antibodies, at least 20,000 antibodies, at least 25,000 antibodies, at least 30,000 antibodies, at least 35,000 antibodies, at least 40,000 antibodies, at least 45,000 antibodies, at least 50,000 antibodies, at least 55,000 antibodies, at least 60,000 antibodies, at least 65,000 antibodies, or at least 70,000 antibodies.

In typical embodiments, the composition comprises a polyclonal antibody. In certain embodiments, the composition comprises polyclonal antibodies.

In some embodiments, the polyclonal antibodies are human antibodies. In typical embodiments, the human antibodies are allogeneic to the recipient. In some embodiments, the polyclonal antibodies are xenogeneic, but have been engineered to reduce, mitigate, or avoid recognition as a xeno-antibody. In certain of these embodiments, the antibodies are humanized antibodies.

In some embodiments, the composition comprises IgG (e.g., IgG1, IgG2, IgG3, IgG4) antibodies. In some embodiments, the composition comprises IgA antibodies.

In certain embodiments, immunoglobulin from pooled human plasma is used. In these embodiments, the immunoglobulin can be pooled from any number of individuals (e.g., donors), including 10 or more individuals, 100 or more individuals, 1000 or more individuals, or more. In certain embodiments, the composition includes a mixture of polyclonal antibodies obtained from at least 100 donors. In certain embodiments, the composition includes a mixture of polyclonal antibodies obtained from at least 500 donors, at least 1000 donors, at least 5000 donors, at least 10,000 donors, at least 15,000 donors, at least donors, at least 25,000 donors, at least 30,000 donors, at least 35,000 donors, at least donors, at least 45,000 donors, at least 50,000 donors, at least 55,000 donors, at least donors, at least 65,000 donors, or at least 70,000 donors.

In some embodiments, the antibodies are isolated or purified from serum prior to use. The purification can be conducted before or after pooling the antibodies from different individuals.

In some embodiments, the pooled immunoglobulin has been determined prior to administration to contain neutralizing antibodies to one or more rAAV capsid serotypes. In some embodiments, the composition comprises within the pool immunoglobulins from an individual or individuals previously determined to contain at least one antibody capable of neutralizing a specific serotype of AAV or an rAAV containing a specific capsid protein.

In some embodiments, the polyclonal composition comprises at least one species of recombinant antibody. In certain embodiments, the composition includes a mixture of recombinant antibodies. In certain embodiments, the mixture of recombinant antibodies includes 2 or more, 3 or more, 4 or more, 5 or more, 6 or more, 7 or more, 8 or more, 9 or more, or 10 or more, 100 or more, 1000 or more, 10,000 or more, or 100, 000 or more species of recombinant antibodies.

In some embodiments, a mixture of recombinant polyclonal antibodies comprises antibodies generated from a library of recombinant constructs containing coding sequences of two or more monoclonal antibodies or antigen binding fragment. In some embodiments, recombinant polyclonal antibodies are generated by mixing and combining antibodies generated from more than one recombinant construct, each encoding an antibody or antigen binding fragment. In some embodiments, recombinant polyclonal antibodies recognize and bind to different epitopes of a single antigen. In some embodiments, recombinant polyclonal antibodies recognize and bind to different antigens.

In certain embodiments, the at least one antibody is a monoclonal antibody. In these latter embodiments, the binding specificity of the monoclonal antibodies is chosen based on its ability to neutralize the administered rAAV.

In some embodiments, the at least one antibody is a monoclonal antibody of a defined sub-class (e.g., IgG1, IgG2, IgG3, IgG4, IgA1, or IgA2). If combinations of antibodies are used, the antibodies can be from the same subclass or from different subclasses. For example, the antibodies can be IgG1 antibodies. Various combinations of different subclasses, in different relative proportions, can be obtained by those of skill in the art.

In some embodiments, the antibody is a monoclonal antibody against an AAV or rAAV containing a capsid protein of an AAV selected from: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

In various embodiments, the antibody is a monoclonal antibody selected from: anti-AAV2 antibody; anti-AAV1 antibody; anti-AAV6 antibody; anti-AAV3 antibody; anti-AAV LK03 antibody; anti-AAV7 antibody; anti-AAV8 antibody; anti-AAV hu.37 antibody; anti-AAV rh.10 antibody; anti-AAV9 antibody; anti-AAV hu.68 antibody; anti-AAV10 antibody; anti-AAV5 antibody; anti-AAV3-3 antibody; anti-AAV4-4 antibody; anti-AAV1-A antibody; anti-hu.46-A antibody; anti-hu.48-A antibody; anti-hu.44-A antibody; anti-hu.43-A antibody; anti-AAV6-A antibody; anti-hu.34-B antibody; anti-hu.47-B antibody; anti-hu.29-B antibody; anti-rh.63-B antibody; anti-hu.56-B antibody; anti-hu.45-B antibody; anti-rh.57-B antibody; anti-rh.35-B antibody; anti-rh.58-B antibody; anti-rh.28-B antibody; anti-rh.51-B antibody; anti-rh.19-B antibody; anti-rh.49-B antibody; anti-rh.52-B antibody; anti-rh.13-B antibody; anti-AAV2-B antibody; anti-rh.20-B antibody; anti-rh.24-B antibody; anti-rh.64-B antibody; anti-hu.27-B antibody; anti-hu.21-B antibody; anti-hu.22-B antibody; anti-hu.23-B antibody; anti-hu.7-C antibody; anti-hu.61-C antibody; anti-rh.56-C antibody; anti-hu. 9-C antibody; anti-hu.54-C antibody; anti-hu.53-C antibody; anti-hu.60-C antibody; anti-hu.55-C antibody; anti-hu.2-C antibody; anti-hu.1-C antibody; anti-hu.18-C antibody; anti-hu.3-C antibody; anti-hu.25-C antibody; anti-hu.15-C antibody; anti-hu.16-C antibody; anti-hu.11-C antibody; anti-hu.10-C antibody; anti-hu.4-C antibody; anti-rh.54-D antibody; anti-rh.48-D antibody; anti-rh.55-D antibody; anti-rh.62-D antibody; anti-AAV7-D antibody; anti-rh.52-E antibody; anti-rh.51-E antibody; anti-hu.39-E antibody; anti-rh.53-E antibody; anti-hu.37-E antibody; anti-rh.43-E antibody; anti-rh.50-E antibody; anti-rh.49-E antibody; anti-rh.61-E antibody; anti-hu.41-E antibody; anti-rh.64-E antibody; anti-hu.42-E antibody; anti-rh.57-E antibody; anti-rh.40-E antibody; anti-hu.67-E antibody; anti-hu.17-E antibody; anti-hu.6-E antibody; anti-hu.66-E antibody; anti-rh.38-E antibody; anti-hu.32-F antibody; anti-AAV9/hu antibody; anti-hu.31-F antibody; anti-Anc80 antibody; anti-Anc81 antibody; anti-Anc82 antibody; anti-Anc83 antibody; anti-Anc84 antibody; anti-Anc94 antibody; anti-Anc113 antibody; anti-Anc126 antibody; anti-Anc127 antibody; anti-Anc80L27 antibody; anti-Anc80L59 antibody; anti-Anc80L60 antibody; anti-Anc80L62 antibody; anti-Anc80L65 antibody; anti-Anc80L33 antibody; anti-Anc80L36 antibody; anti-Anc80L44 antibody; anti-Anc80L1 antibody; anti-Anc110 antibody; and anti-Anc80DI antibody.

In some embodiments, the composition comprises at least one bispecific antibody. In certain embodiments, the bispecific antibody has a binding specificity for at least two different epitopes, at least one of which is an epitope of a rAAV.

In some embodiments, the antibody is a single-domain antibody (dAb). In some embodiments, the antibody is a diabody (db). In some embodiments, the antibody is a nanobody. In some embodiments, the antibody is a unibody, or a diabody.

In some embodiments, the antibody is administered in a second pharmaceutical composition. In some embodiments, the second composition comprises polyclonal antibodies purified from human serum or generated from recombinant constructs.

6.3.2.1. IVIG

In currently preferred embodiments, the composition comprising neutralizing antibodies is intravenous immunoglobulin (IVIG). In certain embodiments, the IVIG is selected from: Gammagard™ Liquid® (Baxter HealthCare Corp), Gammagard™ S/D, Gammaplex™, Bivigam™, Carimune™ NF, Gamunex-C, Gammaked™, Flebogamma™ DIF, Octagam™, and Privigen™.

In some embodiments, recombinant IVIG (rIVIG) generated from recombinant constructs is used. In these embodiments, IVIG is produced in recombinant cells rather than extracted from donor plasma. In these embodiments, primary B cells are collected from thousands of human donors to generate a library of constructs or cell lines expressing rIVIG. In some embodiments of the invention, the library of protein expression constructs is then introduced into a population of cells to produce a library of engineered cells that express a library of hundreds, thousands, or millions of antibody proteins. In some embodiments, these antibody proteins are substantially equivalent to the antibodies produced by the original primary B cells.

In some embodiments, massively parallel DNA sequencing is used to determine the diversity of the B cells, the library of protein expression constructs, and/or the engineered host cells. Methods of making rIVIG can be found in US PreGrant Publication No. 2016/0362470, which is hereby incorporated by reference in its entirety.

In some embodiments, the IVIG or rIVIG is administered in a second pharmaceutical composition.

6.3.2.2. AAV-Neutralizing Antigen Binding Fragments

In some embodiments, the second composition comprises AAV-neutralizing antigen-binding fragments.

In certain embodiments, the second composition comprises a mixture of AAV-neutralizing antigen-binding fragments. In certain embodiments, the mixture comprises 1 or more, 2 or more, 3 or more, 4 or more, 5 or more, 6 or more, 7 or more, 8 or more, 9 or more, or more, 11 or more, 12 or more, 13 or more, 14 or more, 15 or more, 16 or more, 17 or more, 18 or more, 19 or more, 20 or more, 100 or more, 1000 or more, 10,000 or more, or 100,000 or more AAV-neutralizing antigen-binding fragments.

In certain embodiments, the antigen-binding fragment is selected from: a single chain Fv (scFv), a Fab, a (Fab′)₂, and an (ScFv)₂.

In certain embodiments, the antigen-binding fragment is an scFv. In certain embodiments, the antigen-binding fragment is an (ScFv)₂.

In certain embodiments, the antigen-binding fragment is a Fab. In certain embodiments, the antigen-binding fragment is a (Fab′)₂.

In some embodiments, an antibody or antigen-binding fragment thereof is administered in a second composition. In some embodiments, the second composition is a second pharmaceutical composition. In certain embodiments, the second pharmaceutical composition comprises an antibody or antigen-binding fragment thereof that is capable of neutralizing the non-systemically administered rAAV and a pharmaceutically acceptable carrier.

In some embodiments, the second pharmaceutical composition comprises at least 10, at least 100, at least 1000, at least 10,000 or at least 100,000 antigen-binding fragments. In some embodiments, the second pharmaceutical composition comprises at least 10, at least 100, at least 1000, at least 10,000 or at least 100,000 antigen-binding fragments.

6.3.3. Routes and Schedule of Administration

The present disclosure provides a method comprising administering a first pharmaceutical composition comprising a rAAV, and adjunctively administering a second pharmaceutical composition comprising at least one antibody or antigen-binding fragment thereof that is capable of neutralizing the rAAV. The first pharmaceutical composition comprising rAAV is administered non-systemically. The second pharmaceutical composition comprising at least one antibody or antigen-binding fragment thereof that is capable of neutralizing the non-systemically administered rAAV is administered systemically.

In some embodiments, the second pharmaceutical composition comprising the antibody or antigen-binding fragment thereof is administered before administration of the first pharmaceutical composition comprising the rAAV to the patient.

In certain embodiments, the second pharmaceutical composition is administered at least 30 minutes before, at least 1 hour before, at least 2 hours before, at least 4 hours before, at least 6 hours before, at least 8 hours before, at least 10 hours before, at least 12 hours before, at least 14 hours before, at least 16 hours before, at least 18 hours before, at least 20 hours before, at least 22 hours before, or at least 24 hours before, administration of the first pharmaceutical composition to the patient. In certain embodiment, the second pharmaceutical composition is administered at least 1 day before, 2 days before, 3 days before, 4 days before, 5 days before, 6 days before, or 7 days before administration of the first pharmaceutical composition to the patient.

In certain embodiments, the second pharmaceutical composition is administered at least one day before, at least one week before, or at least two weeks before administration of the first pharmaceutical composition to the patient. In some embodiments, the second pharmaceutical composition comprising the antibody or antigen-binding fragment thereof is administered after administration of the first pharmaceutical composition comprising the rAAV to the patient.

In certain embodiments, the second pharmaceutical composition is administered at least 30 minutes after, at least 1 hour after, at least 2 hours after, at least 4 hours after, at least 6 hours after, at least 8 hours after, at least 10 hours after, at least 12 hours after, at least 14 hours after, at least 16 hours after, at least 18 hours after, at least 20 hours after, at least 22 hours after, or at least 24 hours after, administration of the first pharmaceutical composition to the patient. In certain embodiments, the second pharmaceutical composition is administered at 30 minutes after, 1 hour after, 2 hours after, 4 hours after, 6 hours after, 8 hours after, 10 hours after, 12 hours after, 14 hours after, 16 hours after, 18 hours after, 20 hours after, 22 hours after, or 24 hours after, administration of the first pharmaceutical composition to the patient. In certain embodiments, the second pharmaceutical composition is administered at least one day after, at least one week after, at least two weeks after, or at least one month after administration of the first pharmaceutical composition to the patient. In certain embodiments, the second pharmaceutical composition is administered at least one day after, at least two days after, at least three days after, at least four days after, at least five days after, at least six days after, or at least seven days after administration of the first pharmaceutical composition to the patient.

In some embodiments, the first pharmaceutical composition comprising the rAAV and the second pharmaceutical composition comprising the antibody or antigen-binding fragment thereof are administered on the same day.

In some embodiments, the second pharmaceutical composition comprising the antibody or antigen-binding fragment thereof is administered at a sufficient amount to alter biodistribution of the rAAV following administration to the patient. Administration of the second pharmaceutical composition reduces rAAV infection of non-target tissues, cells, and/or organs. In certain embodiments, administration of the second pharmaceutical composition reduces rAAV infection of liver cells, skeletal muscle cells, myocardial cells, lung tissue, or other non-target organ cells outside of the non-systemic compartment into which the rAAV was administered. In certain embodiments, the second pharmaceutical composition is administered in a sufficient amount to reduce liver toxicity or other non-target organ toxicity outside of the non-systemic compartment of the rAAV following administration to the patient. In certain embodiments, the second pharmaceutical composition is administered in a sufficient amount to reduce toxicity in dorsal root ganglion (DRG). In some embodiments, the second pharmaceutical composition is administered at a sufficient amount to reduce systemic toxicity of the rAAV following administration to the patient.

In some embodiments, the method further comprises administering an additional pharmaceutical composition comprising an rAAV. In some embodiments, the additional pharmaceutical composition is administered prior to administration of the first pharmaceutical composition and the second pharmaceutical composition. In some embodiments, the additional pharmaceutical composition is administered after administration of the first pharmaceutical composition and the second pharmaceutical composition. In some embodiments, the additional pharmaceutical composition is administered with a pharmaceutical composition comprising at least one antibody or antigen-binding fragment thereof.

Accordingly, also provided herein are methods comprising administering a third pharmaceutical composition comprising a rAAV, and adjunctively administering a fourth pharmaceutical composition comprising at least one antibody or antigen-binding fragment thereof that is capable of neutralizing the rAAV. The rAAV in the third pharmaceutical composition can be same as or different from the rAAV in the first pharmaceutical composition. In some embodiments, the third pharmaceutical composition is identical to the first pharmaceutical composition. In some embodiments, the third pharmaceutical composition is different from the first pharmaceutical composition. In some embodiments, the fourth pharmaceutical composition is identical to the second pharmaceutical composition. In some embodiments, the fourth pharmaceutical composition is different from the second pharmaceutical composition.

In certain embodiments, a patient having received a first dose of the first pharmaceutical composition comprising a rAAV may need a second dose (a third dose, a fourth dose, or a fifth or more doses) of the first pharmaceutical composition so that a therapeutically effective amount of the rAAV is administered to the patient. In such embodiments, administering the second pharmaceutical composition prior to, contemporaneously with (e.g., on the same day), or after the second dose of the first pharmaceutical compound enables a therapeutically effective amount of the first pharmaceutical composition to be delivered to the patient.

In some embodiments, the second dose of the first pharmaceutical composition is administered to the patient at a lower dose than the first dose of the first pharmaceutical composition administered to the patient. In some embodiments, the second dose of the first pharmaceutical composition is administered to the patient at a higher dose than the first dose of the pharmaceutical composition administered to the patient. In some embodiments, the second dose of the first pharmaceutical composition is administered to the patient at the same dose as the first dose of the pharmaceutical composition administered to the patient.

In some embodiments, the second dose of the second pharmaceutical composition is administered to the patient at a lower dose than the first dose of the second pharmaceutical composition administered to the patient. In some embodiments, the second dose of the second pharmaceutical composition is administered to the patient at a higher dose than the first dose of the second pharmaceutical composition administered to the patient. In some embodiments, the second dose of the second pharmaceutical composition is administered to the patient at the same dose as the first dose of the second pharmaceutical composition administered to the patient.

In some embodiments, the first or second dose of the second pharmaceutical composition is administered before administration (e.g., any of the exemplary timings described herein) of the first or second dose of the first pharmaceutical composition to the patient. In some embodiments, the first or second dose of the second pharmaceutical composition is administered after administration (e.g., any of the exemplary timings described herein) of the first or second dose of the first pharmaceutical composition to the patient. In some embodiments, the first or second dose of the first pharmaceutical composition and the second dose of the second pharmaceutical composition are administered to the patient on the same day.

Also provided herein are methods comprising administering a second, third, fourth, fifth, sixth, seventh, eighth, ninth, tenth, or more doses of a first pharmaceutical composition comprising a rAAV and adjunctively administering a second, third, fourth, fifth, sixth, seventh, eighth, ninth, tenth, or more doses of a second pharmaceutical composition comprising at least one antibody or antigen-binding fragment thereof that is capable of neutralizing the rAAV.

6.3.3.1. Non-Systemic Administration of rAAV

As described herein, rAAV or a first pharmaceutical composition comprising rAAV is administered via a non-systemic route of administration to the patient. In some embodiments, rAAV or a composition comprising the rAAV is administered to the brain, spinal cord, muscle, eye, ear, or any other organ by local or regional delivery.

In some embodiments, rAAV or the first pharmaceutical composition is non-systemically administered to the central nervous system by intrathecal administration, intracerebroventricular administration, or intracisternamagna administration. In certain embodiments, rAAV or the first pharmaceutical composition comprising rAAV is administered to the brain, spinal cord, retina, olfactory epithelium, eye, or ear. In certain embodiments, the target organ is the brain.

In certain embodiments, rAAV or the first pharmaceutical composition is administered to a target tissue selected from the hippocampus, cortex, thalamus (including the central thalamus), sensory cortex, ventral tegmental area (VTA), prefrontal cortex (PFC), nucleus accumbens (NAc), amygdala, substantia nigra, ventral pallidum, global pallidus, dorsal striatum, ventral striatum, subthalamic nucleus, dentate gyrus, cingulate gyms, entorhinal cortex, olfactory cortex, primary motor cortex, and the cerebellum. In certain embodiments, the target tissue in the brain is selected from the group consisting of: hippocampus, cortex, amygdala, and the basal ganglion.

In certain embodiments, the target organ is the eye.

In certain embodiments, the target organ is the muscle.

In some embodiments, rAAV or the first pharmaceutical composition is administered locally (e.g., non-systemic). In certain embodiments, rAAV or the first pharmaceutical composition is administered by intrathecal injection or infusion, intraocular injection, intravitreal injection or infusion, inner ear injection or infusion, intracerebroventricular injection or infusion, intracisternal magna injection (ICM) or infusion, intramuscular injection or infusion, subretinal injection or infusion, or by other routes of non-systemic administration. In certain embodiments, the first pharmaceutical composition is administered by intraarticular injection or infusion, intracardiac injection or infusion, intradermal or intracutaneous injection, or intrathecal injection or infusion.

In certain embodiments, the first pharmaceutical composition is administered by intrathecal injection. In certain embodiments, the first pharmaceutical composition is administered by intrathecal infusion.

In certain embodiments, the first pharmaceutical composition is administered by intraocular injection. In certain embodiments, the first pharmaceutical composition is administered by intraocular infusion.

In certain embodiments, the first pharmaceutical composition is administered by intravitreal injection. In certain embodiments, the first pharmaceutical composition is administered by intravitreal infusion.

In certain embodiments, the first pharmaceutical composition is administered by intracerebroventricular injection. In certain embodiments, the first pharmaceutical composition is administered by intracerebroventricular infusion.

In certain embodiments, the first pharmaceutical composition is administered by subretinal injection or infusion.

In certain embodiments, the first pharmaceutical composition is administered by intracisternal magna injection. In certain embodiments, the first pharmaceutical composition is administered by intracisternal magna infusion.

In certain embodiments, the first pharmaceutical composition is administered by inner ear injection. In certain embodiments, the first pharmaceutical composition is administered by inner ear infusion.

In certain embodiments, the first pharmaceutical composition is administered by intramuscular injection. In certain embodiments, the first pharmaceutical composition is administered by intramuscular infusion.

6.3.3.2. Systemic Administration of Neutralizing Antibodies

In the methods described herein, the second pharmaceutical composition is administered via systemic route of administration. In some embodiments, the second pharmaceutical composition is administered via intramuscular injection, intraosseous injection, intratracheal injection, intravenous injection, or subcutaneous injection. In certain embodiments, systemic route of administration is intravenous injection or infusion. In some embodiments, systemic route of administration is, but is not limited to, or transmucosal, sublingual, or rectal administration.

The second pharmaceutical composition is administered systemically in the present methods for the purpose of neutralizing and/or clearing rAAVs that escape the target tissues and organs and are circulating systemically within the patient. For example, the rAAV can escape to a systemic compartment that includes non-target tissues and organs or a location within the patient outside of the central nervous system.

In some embodiments, neutralizing antibodies or fragments thereof are administered at a dosage ranging from 1-2000 mg/kg patient weight. In certain embodiments, the neutralizing antibodies or fragments thereof are administered at a dose ranging from 1-2 mg/kg, 1-3 mg/kg, 1-5 mg/kg, 1-6 mg/kg, 1-7 mg/kg, 1-8 mg/kg, or 1-9 mg/kg, patient weight. In certain embodiments, the neutralizing antibodies or fragments thereof are administered at a dose of at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least mg/kg, at least 11 mg/kg, at least 12 mg/kg, at least 13 mg/kg, at least 14 mg/kg, at least mg/kg, at least 16 mg/kg, at least 17 mg/kg, at least 18 mg/kg, at least 19 mg/kg, or at least 20 mg/kg, patient weight.

In some embodiments, IVIG is administered at a dose ranging from 1-2000 mg/kg patient weight. In some embodiments, IVIG is administered at a dose ranging from 1-200 mg/kg patient weight. In some embodiments, IVIG is administered at a dose ranging from 1-100 mg/kg patient weight. In some embodiments, IVIG is administered at a dose ranging from 1-50 mg/kg patient weight. In some embodiments, IVIG is administered at a dose ranging from 1-10 mg/kg patient weight. In some embodiments, IVIG is administered at a dose ranging from 1-2 mg/kg patient weight. In some embodiments, IVIG is administered at a dose ranging from 1-10 mg/ml. In some embodiments, IVIG is administered at a dose ranging from 1-4 mg/ml.

In certain embodiments, the neutralizing antibodies or fragments thereof are administered at a dose of at least 20 mg/kg, at least 30 mg/kg, at least 40 mg/kg, at least 50 mg/kg, at least 60 mg/kg, at least 70 kg/mg, at least 80 kg/mg, at least 90 mg/kg, or at least 100 mg/kg, patient weight. In certain embodiments, the neutralizing antibodies or fragments thereof are administered at a dose of at least 110 mg/kg, at least 125 mg/kg, at least 150 mg/kg, at least 175 mg/kg, at least 200 mg/kg, at least 225 mg/kg, at least 250 mg/kg, at least 275 mg/kg, at least 300 mg/kg, at least 325 mg/kg, at least 350 mg/kg, at least 375 mg/kg, at least 400 mg/kg, at least 425 mg/kg, at least 450 mg/kg, at least 475 mg/kg, at least 500 mg/kg, at least 525 mg/kg, at least 550 mg/kg, at least 575 mg/kg, at least 600 mg/kg, at least 625 mg/kg, at least 650 mg/kg, at least 675 mg/kg, at least 700 mg/kg, at least 725 mg/kg, at least 50 mg/kg, at least 775 mg/kg, at least 800 mg/kg, at least 835 mg/kg, at least 850 mg/kg, at least 875 mg/kg, at least 900 mg/kg, at least 925 mg/kg, at least 950 mg/kg, at least 950 mg/kg, at least 975 mg/kg, or at least 1000 mg/kg, patient weight.

In certain embodiments, the neutralizing antibodies or fragments thereof are administered at a dose of at least 1100 mg/kg, at least 1200 mg/kg, at least 1300 mg/kg, at least 1400 mg/kg, at least 1500 mg/kg, at least 1600 mg/kg, at least 1700 mg/kg, at least 1800 mg/kg, at least 1900 mg/kg, or at least 2000 mg/kg, patient weight.

In some embodiments, the neutralizing antibodies or fragments thereof are administered at a dose ranging from 1-10 g, 100 mg-1 g, 1-100 mg, 1-75 mg, 1-50 mg, 1-25 mg, 1-10 mg, 1-5 mg, 1-4 mg, or 1-2 mg. In some embodiments, the second pharmaceutical composition comprises neutralizing antibodies or fragments thereof at a concentration ranging from 1-10 g/ml, 100 mg/ml-1 g/ml, 1-100 mg/ml, 1-75 mg/ml, 1-50 mg/ml, 1-25 mg/ml, 1-10 mg/ml, 1-5 mg/ml, 1-4 mg/ml, or 1-2 mg/ml.

In some embodiments, the second pharmaceutical composition is administered as a single-dose. In some embodiments, the second pharmaceutical composition is administered daily. In some embodiments, the second pharmaceutical composition is administered 1 time a day, 2 times a day, 3 times a day, 4 times a day, 5 times a day, 6 times a day, or 7 times a day. In certain embodiments, the second pharmaceutical composition is administered for no more than 1 week, no more than 6 days, no more than 5 days, no more than 4 days, no more than 3 days, or no more than 2 days.

In certain embodiments, administration of the second pharmaceutical composition takes at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 15 minutes, at least 20 minutes, at least 25 minutes, at least 30 minutes, at least 45 minutes, at least 50 minutes, or at least 60 minutes. In certain embodiments, administration of the second pharmaceutical composition takes 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 45 minutes, 50 minutes, 60 minutes, 70 minutes, 80 minutes, 90 minutes, 100 minutes, 110 minutes, or 120 minutes.

6.3.4. Effective Amounts of Neutralizing Antibodies

In preferred embodiments, an effective amount of an AAV-neutralizing antibody or antigen-binding fragment is adjunctively administered to a patient via systemic administration. An effective amount as provided herein refers to an amount sufficient to reduce biodistribution of AAV or rAAV outside of a target tissue or target organ.

In some embodiments, the effective amount is an amount sufficient to reduce detection of AAV or rAAV in the liver, spleen, cervical DRG, lumbar DRG, heart, and/or kidney. In some embodiments, the effective amount is an amount sufficient to reduce detection of AAV or rAAV in the liver, spleen and/or heart. In some embodiments, the effective amount is an amount sufficient to reduce detection of AAV or rAAV in the DRG.

In some embodiments, the effective amount is an amount sufficient to reduce levels of binding antibodies (BAb) (e.g., IgG and/or IgM) developed to AAV or rAAV in the cerebrospinal fluid (CSF) as compared to levels in the serum. In some embodiments, the effective amount is an amount sufficient to reduce levels of IgM and/or IgG antibodies developed to AAV or rAAV in the CSF as compared to levels in the serum.

Biodistribution of rAAVs can be tested in an experimental animal by measuring presence of rAAV genome, rAAV infection or expression of an expressible polynucleotide. In some embodiments, targeting is measured in a non-human primate (NHP), mice, rats, birds, rabbits, guinea pigs, hamsters, farm animals (including pigs and sheep), dogs, or cats.

In some embodiments, specific and non-specific targeting of rAAVs to tissues or organs can be measured after non-systemic administration of rAAVs, and/or after non-systemic administration of rAAV and systemic administration of the neutralizing antibody or antigen-binding fragment thereof. In some embodiments, targeting of rAAVs is measured after non-systemic administration of rAAV and after systemic administration of an antibody or antigen binding fragment capable of neutralizing the rAAV.

6.4. Kit

In one aspect, the present disclosure provides a kit for combination therapy, comprising: a first pharmaceutical composition comprising a recombinant adeno associated virus (rAAV) as described herein; and a second pharmaceutical composition comprising an AAV-neutralizing antibody or antigen-binding fragment thereof as described herein.

In some embodiments, the antibody or antigen-binding fragments thereof binds to a capsid protein of the rAAV as described in Section 5.3.1.

In some embodiments, the antibody or antigen-binding fragment thereof is an IgG.

In some embodiments, the second pharmaceutical composition is capable of neutralizing the rAAV. In certain embodiments, the second pharmaceutical composition is capable of neutralizing an AAV or rAAV containing a capsid protein of an AAV, selected from AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI, or a variation thereof.

In some embodiments, the second pharmaceutical composition comprises polyclonal antibodies obtained from at least 100 donors. In some embodiments, second pharmaceutical composition comprises polyclonal antibodies obtained from at least 500, at least 1000, at least 5000, or at least 10,000 donors.

In some embodiments, the second pharmaceutical composition comprises intravenous immunoglobulin (IVIG). In some embodiments, the second pharmaceutical composition comprises recombinant intravenous immunoglobulin (rIVIG). In certain embodiments, the IVIG is selected from: Gammagard™ Liquid® (Baxter HealthCare Corp), Gammagard™ S/D, Gammaplex™, Bivigam™, Carimune™ NF, Gamunex-C, Gammaked™, Flebogamma™ DIF, Octagam™, and Privigen™.

In some embodiments, the second pharmaceutical composition comprises recombinant polyclonal antibodies.

In some embodiments, the second pharmaceutical composition comprises an AAV-neutralizing antigen-binding fragment selected from: a single chain Fv (scFv), a Fab, a (Fab″)₂, and an (ScFv)₂. In some embodiments, the second pharmaceutical composition comprises: a bispecific antibody, a single-domain antibody (dAb), a diabody (db), a nanobody, a unibody, and a diabody.

In some embodiments, the rAAV has a capsid protein of an AAV as described in Section 5.3.1.

### 6.5. EXAMPLES

The following examples are provided by way of illustration not limitation.

### 6.5.1. Example 1: Adjunct Administration of IVIG with Non-Systemic Administration of rAAV

This experiment was performed to test the hypothesis that systemic administration of IVIG restricts the biodistribution of ICV administered AAV9.CAG.GFP. In particular, the experiment was designed to evaluate whether IVIG administration prior to ICV administration of AAV9.CAG.GFP limits AAV vector dissemination from the CNS into peripheral organs, such as the liver, heart, and kidney. After ICV administration of AAV, vector is often found in peripheral organs such as the liver, as well as in the DRGs. How vector escapes the CNS is unclear. IVIG, a purified mix of IgG obtained from thousands of healthy donors, contains neutralizing antibodies against many AAV serotypes, including AAV9. The hypothesis was that systemic administration of IVIG prior to ICV administration of AAV9.CAG.GFP will neutralize vector escaping the CNS and prevent transduction of peripheral organs such as the liver. As shown below, this experiment proved that IVIG administration limits vector dissemination from the CNS into peripheral organs.

6.5.1.1. Experimental Design

Mice to be treated were divided into five treatment Groups (Groups 1-5). Group 1 received an ICV saline injection and served as a vehicle control. Group 2 received AAV9.CAG.GFP by ICV injection. Group 3 was administered 20 mg of IVIG (10 mg IVIG/mL blood) by IV injection 2 hour prior to ICV injection of AAV9.CAG.GFP. Group 4 was administered 20 mg of IVIG by IV injection 24 hour prior to ICV injection of AAV9.CAG.GFP. Group 5 was administered the same dose of AAV9.CAG.GFP by IV injection as a control for vector transduction of peripheral organs. Table 1 provides a summary of the experimental design including experimental conditions for Groups 1-5.

TABLE 1
Experimental Design

| Group | | | | | | Dose Level |
|---|---|---|---|---|---|---|
| No. | n | Test Material | IVIG | | Route | (per animal) |
| 1 | 1 | Vehicle Control | — | | ICV | 0 |
| 2 | 5 | AAV9.CAG.GFP | — | | ICV | 5 × 10¹⁰ |
| 3 | 5 | AAV9.CAG.GFP | 2 | h | ICV | 5 × 10¹⁰ |
| 4 | 5 | AAV9.CAG.GFP | 24 | h | ICV | 5 × 10¹⁰ |

| | | | | | |
|---|---|---|---|---|---|
| 5 | 3 | AAV9.CAG.GFP | — | IV | 5 × 10¹⁰ |

Tissues were collected at necropsy (day 14) to assess vector biodistribution (AAV genomic DNA) and eGFP expression (eGFP mRNA). In addition, blood was collected prior to vector administration (day 0) to validate IVIG administration via neutralizing antibody (NAb) titer assay. Blood and CSF were also collected at necropsy. Upon harvesting, tissue was immediately placed into the preservative RNAlater, after which the RNAlater was removed and the tissue flash frozen or placed into lysis buffer containing proteinase K for DNA isolation. Additional tissue material from the same tissues was fixed and embedded for sectioning and anti-GFP staining by immunohistochemistry (IHC).

6.5.1.2. Neutralizing Antibody Titer Analysis

Neutralizing antibody (NAb) titer analysis was performed on serum from study animals using an in vitro NAb assay. AAV NAb testing was performed by the Immunology Core at the Gene Therapy Program of the University of Pennsylvania as previously described (Calcedo et al. Human Gene Ther Methods. 2018 Apr.; 29(2): 86-95, which is herein incorporated by reference in its entirety).

As shown in Table 1, the mice did not have preexisting antibodies to AAVs and were seronegative prior to study enrollment. On the day of vector administration (Day 1, 2 hour, or 24 hour post-IVIG administration in applicable groups) serum samples were collected prior to vector administration and evaluated for NAbs. The NAb titers are shown in Table 2. Animals administered IVIG, Animal ID #7-#16 had similar NAb titers to IVIG controls (fresh IVIG samples not administered to animals). As expected, animals not administered IVIG had NAb titers below the threshold of detection.

TABLE 2
Levels of anti-AAV9 Neutralizing Antibody Prior to Vector Dosing

| | | | | | AAV9 NAb₅₀ |
|---|---|---|---|---|---|
| | | | | | Titer in |
| | | | | Sample | HEK293 |
| Animal # | Vector Treatment | IVIG | | Type | cells^{1,2} |
| MR-18-2 | AAV9.CAG.GFP, ICV | — | | Serum | <10* |
| MR-18-3 | AAV9.CAG.GFP, ICV | — | | Serum | <5 |
| MR-18-4 | AAV9.CAG.GFP, ICV | — | | Serum | <10* |
| MR-18-5 | AAV9.CAG.GFP, ICV | — | | Serum | <5 |
| MR-18-6 | AAV9.CAG.GFP, ICV | — | | Serum | <5 |
| MR-18-7 | AAV9.CAG.GFP, ICV | 2 | h | Serum | 320 |
| MR-18-8 | AAV9.CAG.GFP, ICV | 2 | h | Serum | 160 |
| MR-18-9 | AAV9.CAG.GFP, ICV | 2 | h | Serum | 40 |
| MR-18-10 | AAV9.CAG.GFP, ICV | 2 | h | Serum | 80 |
| MR-18-11 | AAV9.CAG.GFP, ICV | 2 | h | Serum | 160 |
| MR-18-12 | AAV9.CAG.GFP, ICV | 24 | h | Serum | 80 |
| MR-18-13 | AAV9.CAG.GFP, ICV | 24 | h | Serum | 80 |
| MR-18-14 | AAV9.CAG.GFP, ICV | 24 | h | Serum | 80 |
| MR-18-15 | AAV9.CAG.GFP, ICV | 24 | h | Serum | 160 |
| MR-18-16 | AAV9.CAG.GFP, ICV | 24 | h | Serum | 80 |

| | | | | |
|---|---|---|---|---|
| MR-18-17 | AAV9.CAG.GFP, IV | — | Serum | <40** |
| MR-18-18 | AAV9.CAG.GFP, IV | — | Serum | <10* |
| MR-18-19 | AAV9.CAG.GFP, IV | — | Serum | <10* |
| — | — | — | IVIG | 160 |
| — | — | — | IVIG | 320 |

¹MR-18-7 to MR-18-16 and IVIG sample types (bottom two rows) had positive NAb responses and MR-18-2 to MR-18-6 and MR-18-17 to MR-18-19 had negative NAb responses.
²Values are the serum reciprocal dilution at which relative luminescence units (RLUs) were reduced 50% compared to virus control wells (no test sample). Limit of detection ⅕ dilution.
*Limit of detection 1/10 dilution- not enough serum available for ⅕ dilution.
**Limit of detection 1/40 dilution- not enough serum available for ⅕ dilution.

6.5.1.3.Binding Antibody Titer Analysis

Murine B cell responses were evaluated in serum and CSF using an in vitro Binding antibody (BAb) ELISA. BAb titer analysis was performed on serum and cerebrospinal fluid from study animals using the in vitro BAb ELISA assay at day 14 (necropsy). AAV9 BAb testing was performed as previously described (Calcedo et al. Human Gene Ther Methods. 2018 April; 29(2): 86-95), using anti-mouse IgM-HRP (Invitrogen, Cat. No. 31456) or anti-mouse IgG-HRP (Promega, Cat. No. W402B). Groups were pooled for analysis of IgG in serum and CSF.

IgM BAb titers are shown in Table 3 for individual animals.

TABLE 3
Mouse IgM Bab Titer 14 days Post-Dosing

| | | | | | IgM BAb |
|---|---|---|---|---|---|
| Sample | Groups | Vector Treatment | IVIG | | Titer (1:X) |
| 1 | 1 | Saline | — | | 20 |
| 2 | 2 | AAV9.CAG.GFP, ICV | — | | >1280 |
| 3 | 2 | AAV9.CAG.GFP, ICV | — | | 640 |
| 4 | 2 | AAV9.CAG.GFP, ICV | — | | 640 |
| 5 | 2 | AAV9.CAG.GFP, ICV | — | | 640 |
| 6 | 2 | AAV9.CAG.GFP, ICV | — | | ND |
| 7 | 3 | AAV9.CAG.GFP, ICV | 2 | h | 160 |
| 8 | 3 | AAV9.CAG.GFP, ICV | 2 | h | 160 |
| 9 | 3 | AAV9.CAG.GFP, ICV | 2 | h | 320 |
| 10 | 3 | AAV9.CAG.GFP, ICV | 2 | h | 40 |
| 11 | 3 | AAV9.CAG.GFP, ICV | 2 | h | ND |
| 12 | 4 | AAV9.CAG.GFP, ICV | 24 | h | 160 |
| 13 | 4 | AAV9.CAG.GFP, ICV | 24 | h | 80 |
| 14 | 4 | AAV9.CAG.GFP, ICV | 24 | h | 80 |
| 15 | 4 | AAV9.CAG.GFP, ICV | 24 | h | 160 |
| 16 | 4 | AAV9.CAG.GFP, ICV | 24 | h | 80 |

| | | | | |
|---|---|---|---|---|
| 17 | 5 | AAV9.CAG.GFP, IV | — | 320 |
| 18 | 5 | AAV9.CAG.GFP, IV | — | 40 |
| 19 | 5 | AAV9.CAG.GFP, IV | — | 40 |

IgG BAb titers are shown in Table 4 from pooled groups.

TABLE 4
Mouse IgG BAb Titers 14 Days Post-Dosing

| | | | Sample | IgG BAb |
|---|---|---|---|---|
| Group # | Vector Treatment | IVIG | Type | Titer (1:X) |
| 1 | Saline | — | Serum | 20 |
| 2 | AAV9.CAG.GFP, ICV | — | Serum | 64000 |
| 3 | AAV9.CAG.GFP, ICV | — | Serum | 64000 |
| 4 | AAV9.CAG.GFP, ICV | — | Serum | 32000 |
| 5 | AAV9.CAG.GFP, ICV | — | Serum | >1280 |
| 1 | AAV9.CAG.GFP, ICV | — | CSF | <100 |
| 2 | AAV9.CAG.GFP, ICV | 2 h | CSF | 400 |
| 3 | AAV9.CAG.GFP, ICV | 2 h | CSF | 200 |
| 4 | AAV9.CAG.GFP, ICV | 2 h | CSF | 100 |
| 5 | AAV9.CAG.GFP, ICV | 2 h | CSF | 400 |

As shown in Tables 3 and 4, mice treated with IVIG prior to dosing of AAV developed lower levels of IgM and/or IgG antibodies to AAV in the CSF as compared to levels of IgM and/or IgG antibodies in the serum. This suggests that IVIG protects the CNS from developing high levels of IgM and/or IgG antibodies to administered AAV capsids.

6.5.1.4. Vector Genome Biodistribution

Vector genome distribution (i.e., AAV vector genomic DNA) in various tissues was measured by ddPCR and presented as the ratio of eGFP DNA copy number over RPP30 DNA copy number (eGFP/RPP30), a rough indicator of vector genome copies per cell. Tissues were harvested 14 days after AAV9.CAG.GFP injection and DNA was isolated. DNA was analyzed using the Bio-Rad ddPCR Supermix for Probes (no dUTP) (Bio-Rad 1863024) in combination with primers and probes specific for DNA encoding the eGFP transgene and DNA encoding the non-human primate RPP30 reference. Primers and probes were designed to include intronic sequences to prevent contaminating RNA from interfering with accurate quantitation of vector genomes. After thermal cycling, samples were analyzed on the Bio-Rad QX200 Droplet Reader instrument using the Absolute Quantitation program.

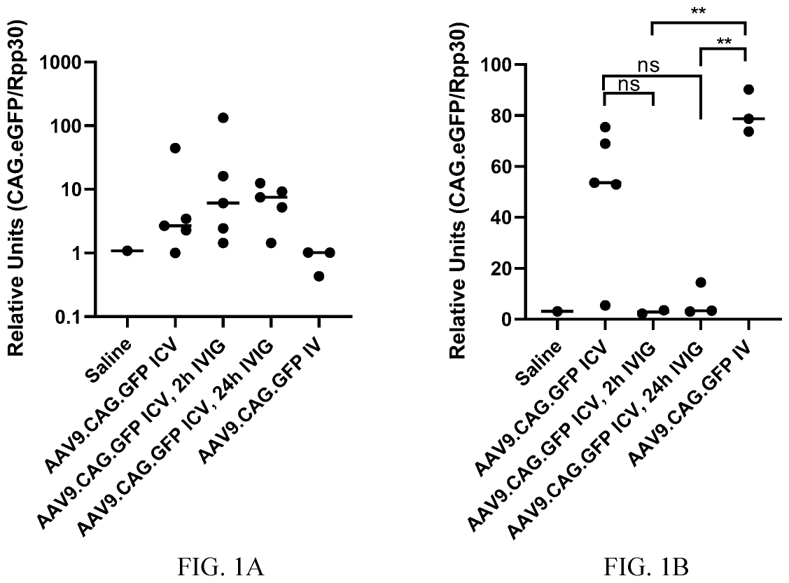

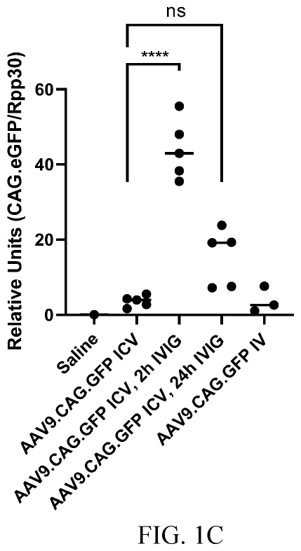

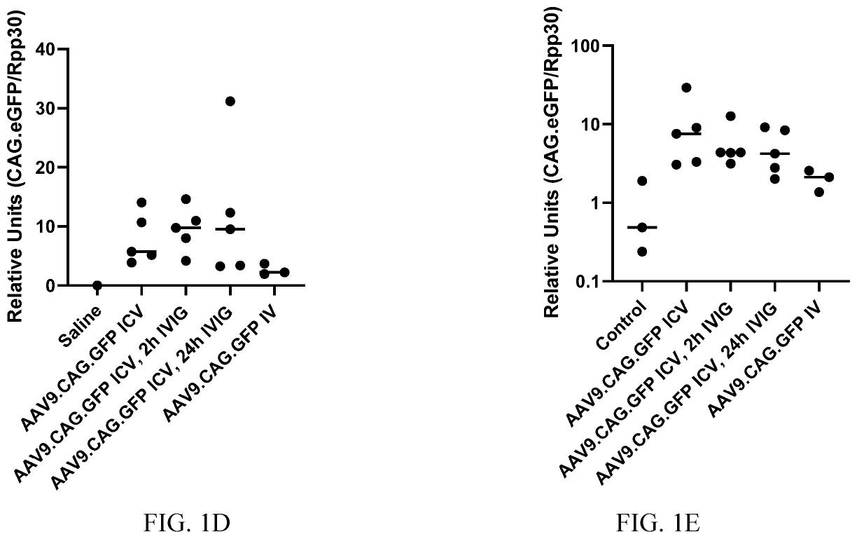

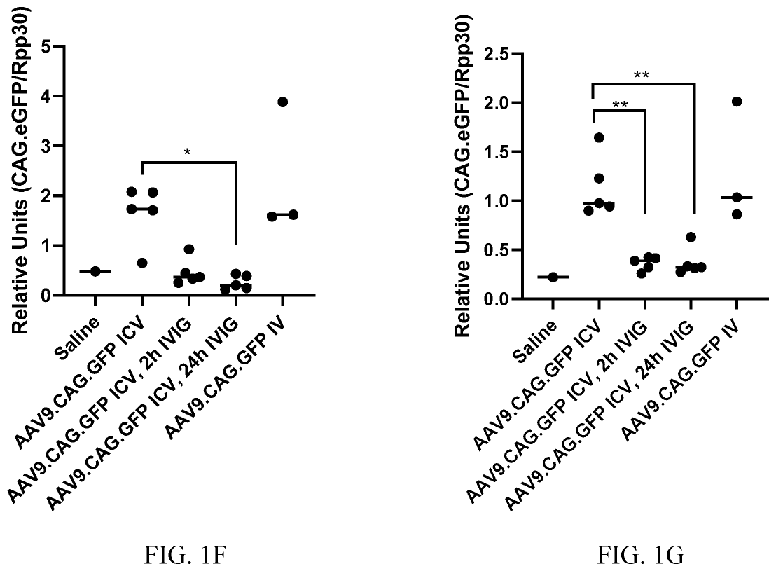

The results of the vector genome biodistribution measurements are provided in FIGS. 1A (brain), 2B (liver), 2C (spleen), 2D (cervical DRG), 2E (lumbar DRG), 2F (heart), and 2G (Kidney). For example, the copies (cp)/μL quantities for the DNA encoding the eGFP transgene were normalized to the cp/μL quantities of the DNA encoding the RPP30 reference and multiplied by 100 to generate a ratio of eGFP DNA copy number over RPP30 DNA copy number. For each tissue, results from three biological replicates were provided for each treatment. Statistically significant differences were determined by an ANOVA 1-way test with P-values and indicated with asterisks. * P<0.05, ** p<0.002; *** P<0.001; **** p<0.0001, ns=not significant.

FIG. 1A shows the ratio of eGFP to RPP30 DNA copy number in the brain. No significant difference was observed between any treatment groups.

FIG. 1B shows the ratio of eGFP to RPP30 DNA copy number in the liver. IVIG administration 2 hours or 24 hours prior to AAV9.CAG.GFP ICV administration resulted in a decrease in vector transduction.

FIG. 1C shows the ratio of eGFP to RPP30 DNA copy number in the spleen. Animals dosed with AAV9 via tail vein IV-injection had similar copies of vector genomes compared to ICV-injected controls, suggesting that vector is escaping the CNS into circulation. Animals administered IVIG 2 hours prior to AAV9.CAG.GFP ICV administration led to a significant increase in vector genomes, while the group administered IVIG 24 hours prior to AAV9.CAG.GFP ICV administration displayed an elevated but not significant increase in vector genomes. This is possibly due to clearance or degradation of some IVIG in the circulation prior vector administration in the 24 h treatment. This result is expected, as antibody-bound capsid is targeted for engulfment by phagocytic cells in the circulation. Thus, this increase in vector genomes in the spleen is likely inactivated AAV vector in macrophages, neutrophils, and dendritic cell populations present in the spleen at the time of necropsy (day 14).

FIGS. 1D-1E shows the ratio of eGFP to RPP30 DNA copy number in cervical DRGs (FIG. 1D) and lumbar DRGs (FIG. 1E).

FIG. 1F shows the ratio of eGFP to RPP30 DNA copy number in the heart. Animals administered AAV9.CAG.GFP via ICV or IV injection had similar vector transduction of heart muscle. Animals administered IVIG either 2 hours or 24 hours prior to AAV9.CAG.GFP ICV administration had reduced vector transduction (i.e., fewer vector genome copies per cell) in the heart, which was statistically significantly at 24 hours.

FIG. 1G shows the ratio of eGFP to RPP30 DNA copy number in the kidney. Animals administered AAV9.CAG.GFP via ICV or IV had similar levels of vector transduction. Animals administered IVIG either 2 hours or 24 hours prior to AAV9.CAG.GFP ICV administration had significantly reduced vector transduction (i.e., fewer vector genome copies per cell) in the kidney.

6.5.1.5. Gene Expression

eGFP mRNA expression in various tissues was measured by RT-ddPCR and presented as the ratio of eGFP transcripts over RPP30 transcripts (eGFP/RPP30), a rough indicator of eGFP mRNA copies per cell. Tissues were harvested 14 days after injection and RNA was isolated. RNA from samples were analyzed using the Bio-Rad One-Step RT-ddPCR Advanced Kit for Probes (Bio-Rad 1864022) in combination with primers and probes specific for the eGFP transgene and the non-human primate RPP30 reference gene. The reverse primer for both targets acted as the reverse transcription primer for the reverse transcription step. Where possible, primers and probes were designed across exon-exon junctions to prevent cross-reactivity with contaminating DNA. After thermal cycling, samples were analyzed on the QX200 Droplet Reader instrument using the Absolute Quantitation program.

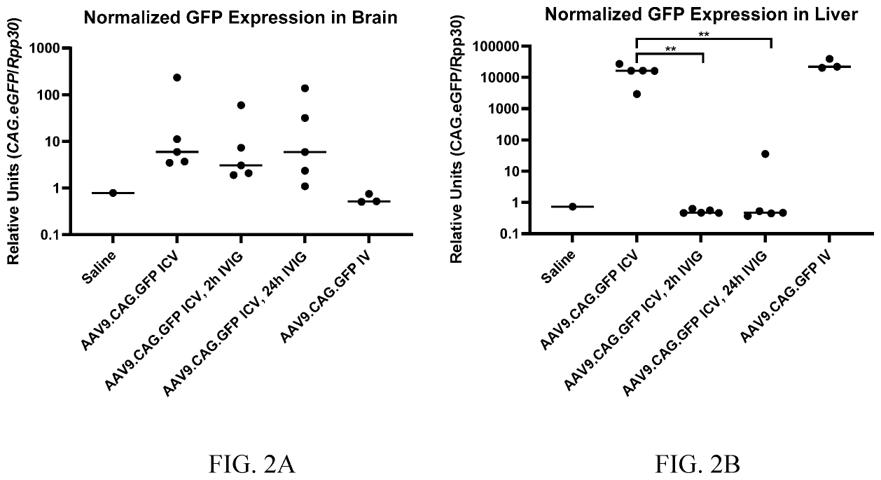

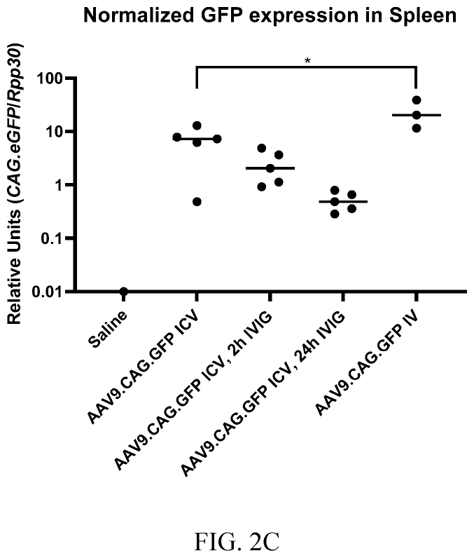

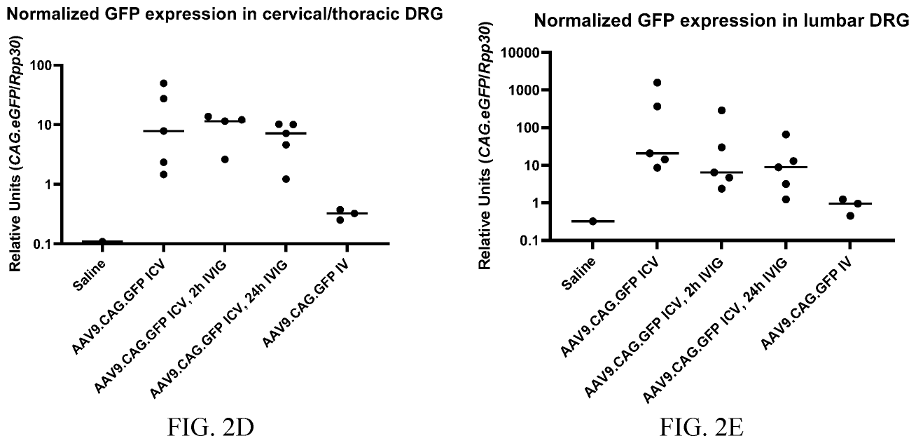

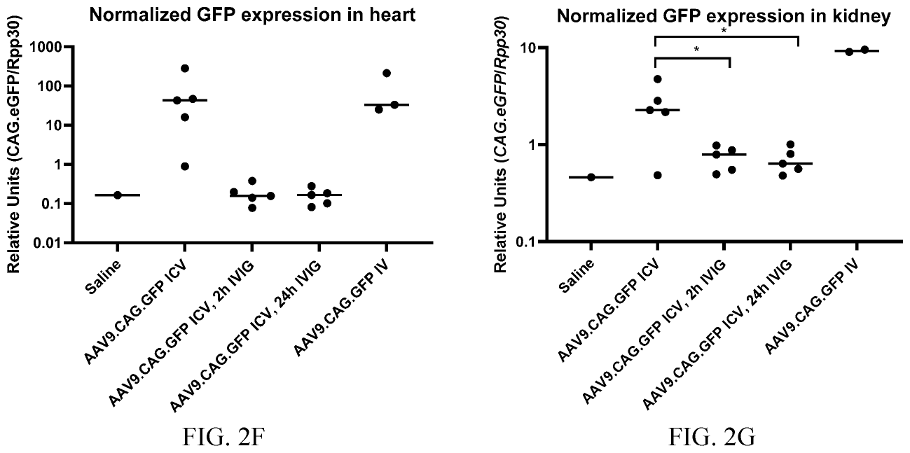

The results of the eGFP mRNA expression measurements are provided in FIGS. 2A (brain), 2B(liver), 2C (spleen), 2D (cervical/thoracic DRG), 2E (lumbar DRG), 2F (heart), and 2G (Kidney). For example, the cp/μL quantities for the eGFP transgene were normalized to the cp/μL quantities of the RPP30 reference gene and multiplied by 100 to generate a ratio of eGFP transcripts over RPP30 transcripts (eGFP/RPP30). For each tissue, results from three biological replicates were provided for each condition. Statistically significant differences were determined by an ANOVA 1-way test with P-values and indicated with asterisks. * P<0.05, ** p<0.002; *** P<0.001; **** p<0.0001, ns=not significant.

FIG. 2A shows the ratio of eGFP to RPP30 transcripts in the brain. Animals treated with IVIG showed no difference in eGFP mRNA expression compared to animals dosed with AAV9.CAG.GFP via ICV administration.

FIG. 2B shows the ratio of eGFP to RPP30 transcripts in the liver. Animals dosed with AAV9.CAG.GFP via ICV displayed similar levels of eGFP mRNA expression to animals dosed with AAV9.CAG.GFP via IV tail vein injection. Animals administered IVIG either 2 hours or 24 hours prior to AAV9.CAG.GFP ICV administration showed significantly reduced eGFP mRNA expression. This expression data is in line with the vector genome biodistribution data.

FIG. 2C shows the ratio of eGFP to RPP30 transcripts in the spleen. Animals dosed with AAV9.CAG.GFP by ICV administration had reduced expression of GFP compared to animals dosed with AAV9.CAG.GFP by IV administration. Animals administered IVIG either 2 hours or 24 hours prior to AAV9.CAG.GFP ICV administration showed slight reduction in GFP expression compared to the ICV-only dosed group, but the reduction was not statistically significant.

FIGS. 2D-2E show the ratio of eGFP to RPP30 transcripts in cervical/thoracic DRG (FIG. 2D) and lumbar DRG (FIG. 2E). In both cervical/thoracic and lumbar DRG groups, animals dosed with AAV9.CAG.GFP via ICV administration showed no difference in expression of eGFP mRNA compared to animals treated with IVIG prior to AAV9.CAG.GFP ICV administration. Animals dosed with AAV9.CAG.GFP via ICV administration do have slightly increased (but not statistically significant) GFP expression compared to animals dosed with AAV9.CAG.GFP via IV administration.

FIG. 2F shows the ratio of eGFP to RPP30 transcripts in the heart. Animals administered IVIG either 2 hours or 24 hours prior to AAV9.CAG.GFP ICV administration showed reduced, but not statistically significant, GFP expression.

FIG. 2G shows the ratio of eGFP to RPP30 transcripts in the kidney. Animals administered IVIG either 2 hours or 24 hours prior to AAV9.CAG.GFP ICV administration showed significantly reduced GFP expression.

6.5.1.6. Protein Expression

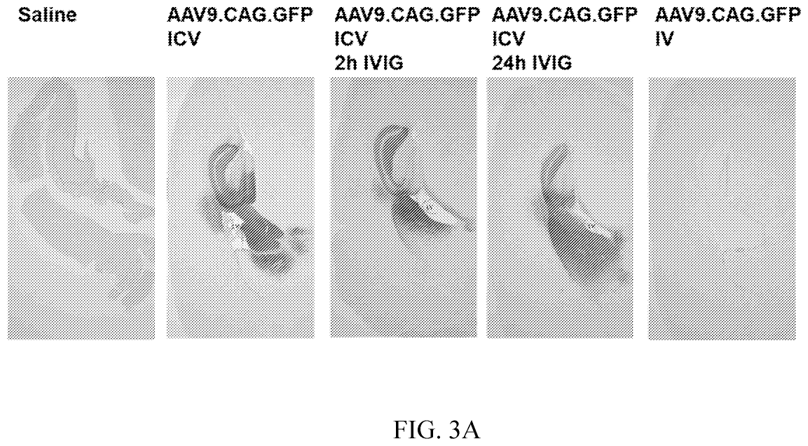

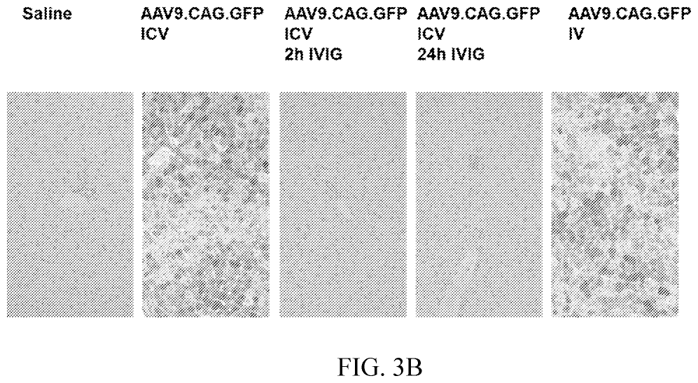

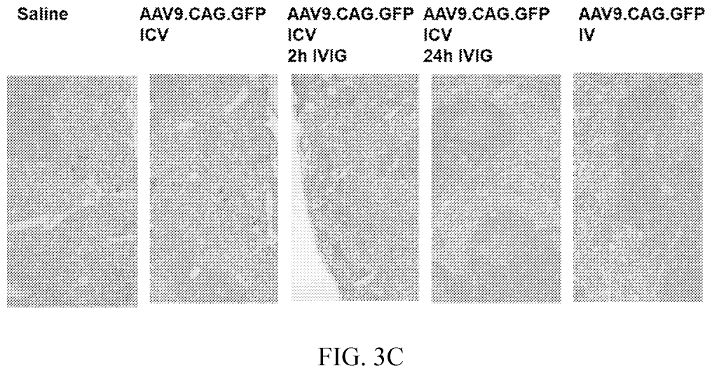

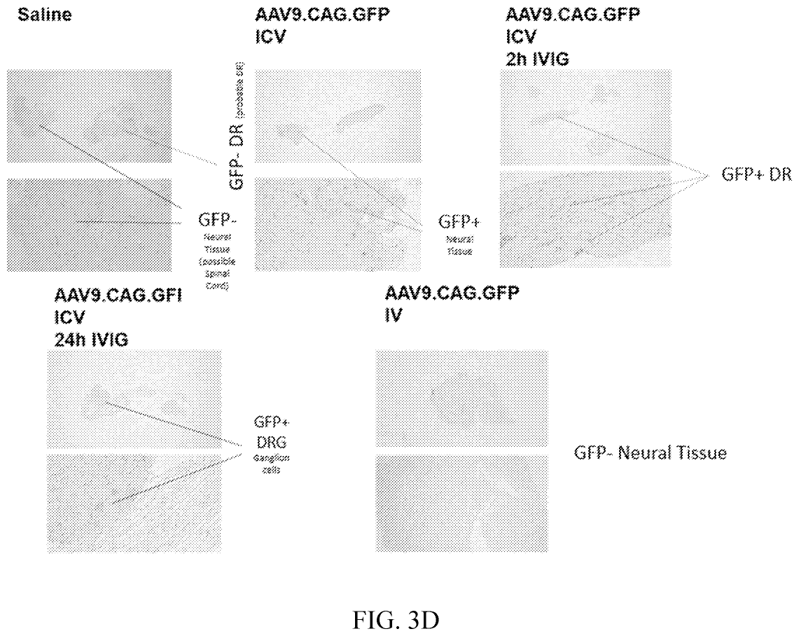

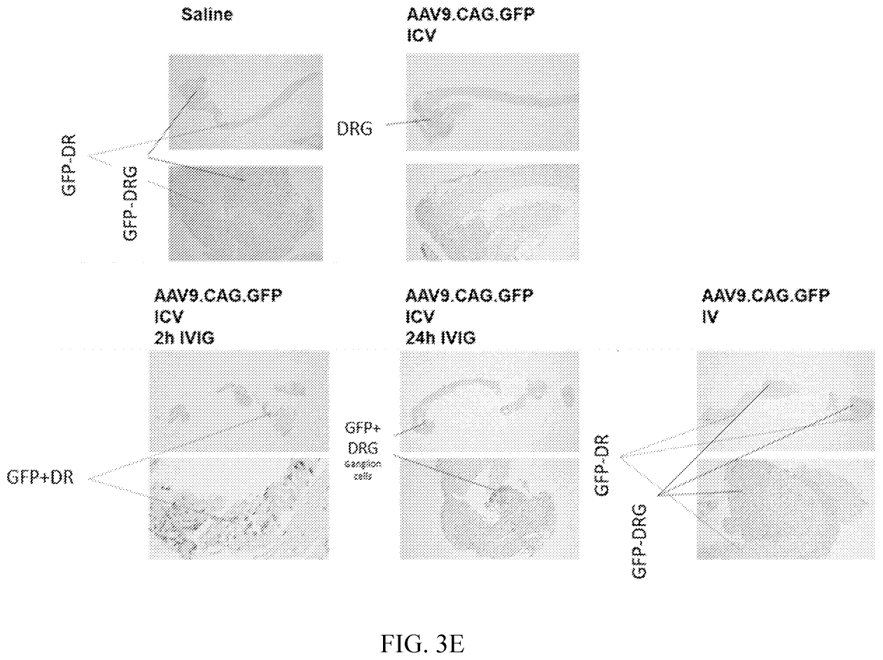

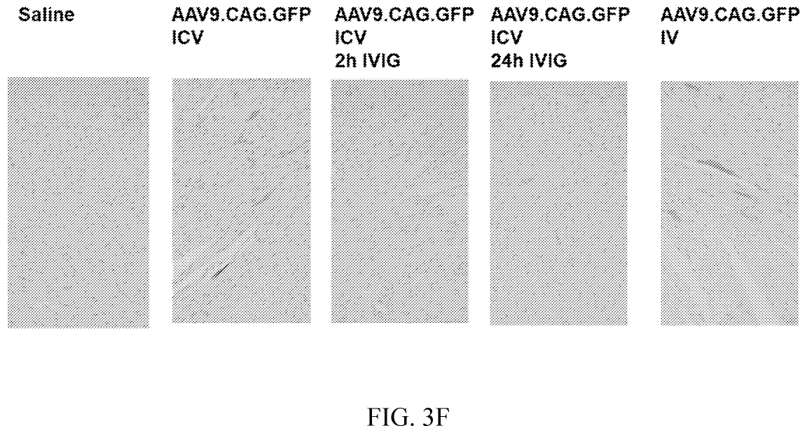

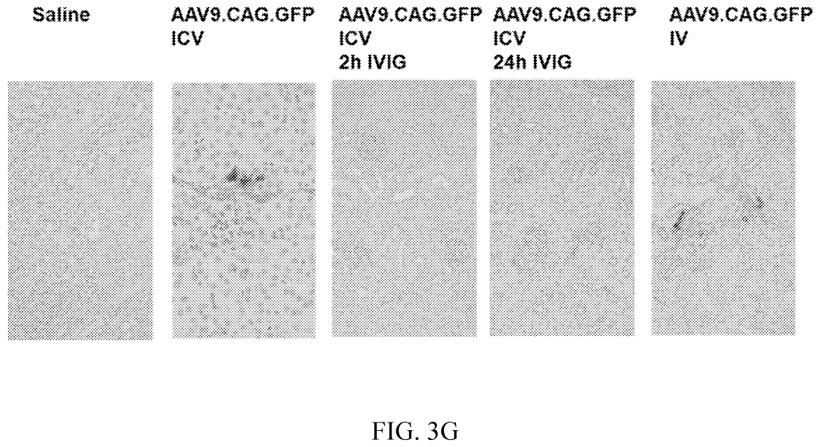

Tissue samples for IHC analysis were collected at day 14 post vector administration and immediately placed into 10% neutral buffered formalin for approximately 48 hours and then transferred to 70% ethanol. Samples in ethanol were shipped at ambient temperature to Histoserv (Germantown, MD). Briefly, tissue samples were trimmed and embedded in paraffin. The resulting blocks were sectioned via microtome, mounted to glass slides, and stained for detection of GFP. Briefly, slides were baked for 15 minutes at 55-65° C. to help remove paraffin and stained using GeneTex, GTX20290 GFP antibody at 1:1,000 in Monet Blue diluent and an anti-rabbit HRP labelled secondary antibody (Biocare Medical, BRR4009). Betazoid DAB (Biocare Medical, BDB2004) was used for development and Mayer's Hematoxylin (StatLab, HXMMHPT) for counterstaining. Representative images from each condition (i.e., saline; AAV9.CAG.GFP ICV; AAV9.CAG.GFP ICV with 2-hour IVIG; AAV9.CAG.GFP with 24 hour IVIG; or AAV9.CAG.GFP IV) are provided in FIGS. 3A (brain), 3B(liver), 3C (spleen), 3D (cervical/thoracic DRG), 3E (lumbar DRG), 3F (heart), and 3G (Kidney).

FIG. 3A shows representative images of brain cross-sections obtained after anti-GFP IHC. FIG. 3A shows eGFP expression in the brains of ICV-dosed animals, as expected. There was little to no expression of eGFP in brains dosed via IV injection, and no background in the saline control. Table 5 provides a summary of percentage of GFP⁺ brain cells for each animal.

TABLE 5
Percentage of GFP⁺ Brain Cells

| | | | | GFP % + | |
|---|---|---|---|---|---|
| Animal # | Vector Treatment | IVIG* | | Cells | −Control |
| MR-18-1 | Saline | — | | 0 | 0 |
| MR-18-2 | AAV9.CAG.GFP, ICV | — | | 5 | 0 |
| MR-18-3 | AAV9.CAG.GFP, ICV | — | | 15 | 0 |
| MR-18-4 | AAV9.CAG.GFP, ICV | — | | 2 | 0 |
| MR-18-5 | AAV9.CAG.GFP, ICV | — | | 7 | 0 |
| MR-18-6 | AAV9.CAG.GFP, ICV | — | | 17 | 0 |
| MR-18-7 | AAV9.CAG.GFP, ICV | 2 | h | 3 | 0 |
| MR-18-8 | AAV9.CAG.GFP, ICV | 2 | h | 2 | 0 |
| MR-18-9 | AAV9.CAG.GFP, ICV | 2 | h | 12 | 0 |
| MR-18-10 | AAV9.CAG.GFP, ICV | 2 | h | 1 | 0 |
| MR-18-11 | AAV9.CAG.GFP, ICV | 2 | h | 8 | 0 |
| MR-18-12 | AAV9.CAG.GFP, ICV | 24 | h | 3 | 0 |
| MR-18-13 | AAV9.CAG.GFP, ICV | 24 | h | 4 | 0 |
| MR-18-14 | AAV9.CAG.GFP, ICV | 24 | h | 4 | 0 |
| MR-18-15 | AAV9.CAG.GFP, ICV | 24 | h | 13 | 0 |
| MR-18-16 | AAV9.CAG.GFP, ICV | 24 | h | 14 | 0 |

| | | | | |
|---|---|---|---|---|
| MR-18-17 | AAV9.CAG.GFP, IV | — | 0+ | 0 |
| MR-18-18 | AAV9.CAG.GFP, IV | — | 0 | 0 |
| MR-18-19 | AAV9.CAG.GFP, IV | — | 0 | 0 |

*MR-18-7 to MR-18-16 had positive NAb responses and MR-18-2 to MR-18-6 and MR-18-17 to MR-18-19 had negative NAb responses.

FIG. 3B shows representative images of liver cross-sections obtained after anti-GFP IHC. GFP expression was evident in groups dosed with AAV alone, either ICV or IV injection. In animals administered IVIG either 2 hours or 24 hours prior to AAV9.CAG.GFP ICV administration, no GFP expression was detected in the liver, which was similar to saline injected control. One exception is animal #15, in the 24-hour IVIG treatment group, which had few GFP positive cells (See Table 6). This animal also had a higher amount of GFP expression compared to the other animals in the group as measured by RT-ddPCR. Table 6 provides a summary of percentage of GFP⁺ liver cells for each animal.

TABLE 6
Percentage of GFP⁺ Liver Cells

| Animal # | Vector Treatment | IVIG* | | Tissue | % GFP + Cells |
|---|---|---|---|---|---|
| MR-18-1 | Saline | — | | Liver | 0 |
| MR-18-2 | AAV9.CAG.GFP, ICV | — | | Liver | 94 |
| MR-18-3 | AAV9.CAG.GFP, ICV | — | | Liver | 60 |
| MR-18-4 | AAV9.CAG.GFP, ICV | — | | Liver | 72 |
| MR-18-5 | AAV9.CAG.GFP, ICV | — | | Liver | 75 |
| MR-18-6 | AAV9.CAG.GFP, ICV | — | | Liver | 88 |
| MR-18-7 | AAV9.CAG.GFP, ICV | 2 | h | Liver | 0 |
| MR-18-8 | AAV9.CAG.GFP, ICV | 2 | h | Liver | 0 |
| MR-18-9 | AAV9.CAG.GFP, ICV | 2 | h | Liver | 0 |
| MR-18-10 | AAV9.CAG.GFP, ICV | 2 | h | Liver | 0 |
| MR-18-11 | AAV9.CAG.GFP, ICV | 2 | h | Liver | 0 |
| MR-18-12 | AAV9.CAG.GFP, ICV | 24 | h | Liver | 0+ |
| MR-18-13 | AAV9.CAG.GFP, ICV | 24 | h | Liver | 0 |
| MR-18-14 | AAV9.CAG.GFP, ICV | 24 | h | Liver | 0 |
| MR-18-15 | AAV9.CAG.GFP, ICV | 24 | h | Liver | 2 |
| MR-18-16 | AAV9.CAG.GFP, ICV | 24 | h | Liver | 0 |

| | | | | |
|---|---|---|---|---|
| MR-18-17 | AAV9.CAG.GFP, IV | — | Liver | 91 |
| MR-18-18 | AAV9.CAG.GFP, IV | — | Liver | 92 |
| MR-18-19 | AAV9.CAG.GFP, IV | — | Liver | 93 |

*MR-18-7 to MR-18-16 had positive NAb responses and MR-18-2 to MR-18-6 and MR-18-17 to MR-18-19 had negative NAb responses.

FIG. 3C shows representative images of spleen cross-sections obtained after anti-GFP IHC. No difference in GFP expression was detected between any treatment groups. Due to the nature of the spleen, a high degree of background is present in saline-injected animals. Table 7 provides a summary of percentage of GFP⁺ spleen cells for each animal.

TABLE 7
Percentage of GFP⁺ Spleen Cells

| | | | | GFP % + | |
|---|---|---|---|---|---|
| Animal # | Vector Treatment | IVIG* | | Cells | −Control |
| MR-18-1 | Saline | — | | 0 | 0 |
| MR-18-2 | AAV9.CAG.GFP, ICV | — | | 0 | 0 |
| MR-18-3 | AAV9.CAG.GFP, ICV | — | | 0 | 0 |
| MR-18-4 | AAV9.CAG.GFP, ICV | — | | 0 | 0 |
| MR-18-5 | AAV9.CAG.GFP, ICV | — | | 0 | 0 |
| MR-18-6 | AAV9.CAG.GFP, ICV | — | | 0 | 0 |
| MR-18-7 | AAV9.CAG.GFP, ICV | 2 | h | 0 | 0 |
| MR-18-8 | AAV9.CAG.GFP, ICV | 2 | h | 0 | 0 |
| MR-18-9 | AAV9.CAG.GFP, ICV | 2 | h | 0 | 0 |
| MR-18-10 | AAV9.CAG.GFP, ICV | 2 | h | 0 | 0 |
| MR-18-11 | AAV9.CAG.GFP, ICV | 2 | h | 0 | 0 |
| MR-18-12 | AAV9.CAG.GFP, ICV | 24 | h | 0 | 0 |
| MR-18-13 | AAV9.CAG.GFP, ICV | 24 | h | 0 | 0 |
| MR-18-14 | AAV9.CAG.GFP, ICV | 24 | h | 0 | 0 |
| MR-18-15 | AAV9.CAG.GFP, ICV | 24 | h | 0 | 0 |
| MR-18-16 | AAV9.CAG.GFP, ICV | 24 | h | 0 | 0 |

| | | | | |
|---|---|---|---|---|
| MR-18-17 | AAV9.CAG.GFP, IV | — | 0+ | 0 |
| MR-18-18 | AAV9.CAG.GFP, IV | — | 0 | 0 |
| MR-18-19 | AAV9.CAG.GFP, IV | — | 0 | 0 |

*MR-18-7 to MR-18-16 had positive NAb responses and MR-18-2 to MR-18-6 and MR-18-17 to MR-18-19 had negative NAb responses.

FIGS. 3D-3E show representative images of cervical/thoracic DRG cross-sections (FIG. 3D) and lumbar DRG cross-sections (FIG. 3E) obtained after anti-GFP IHC. Table 8 and Table 9 provides a summary of percentage of GFP⁺ cells in cervical/thoracic DRG (Table 8) and lumbar DRG (Table 9).

TABLE 8
Percentage of GFP⁺ Cervical/Thoracic DRG

| | | | | GFP % + | |
|---|---|---|---|---|---|
| Animal # | Vector Treatment | IVIG* | | Cells | −Control |
| MR-18-1 | Saline | — | | 0 | 0 |
| MR-18-2 | AAV9.CAG.GFP, ICV | — | | 0 | 0 |
| MR-18-3 | AAV9.CAG.GFP, ICV | — | | + | 0 |
| MR-18-4 | AAV9.CAG.GFP, ICV | — | | + | 0 |
| MR-18-5 | AAV9.CAG.GFP, ICV | — | | 0 | 0 |
| MR-18-6 | AAV9.CAG.GFP, ICV | — | | 0 | 0 |
| MR-18-7 | AAV9.CAG.GFP, ICV | 2 | h | + | 0 |
| MR-18-8 | AAV9.CAG.GFP, ICV | 2 | h | + | 0 |
| MR-18-9 | AAV9.CAG.GFP, ICV | 2 | h | + | 0 |
| MR-18-10 | AAV9.CAG.GFP, ICV | 2 | h | + | 0 |
| MR-18-11 | AAV9.CAG.GFP, ICV | 2 | h | 0 | 0 |
| MR-18-12 | AAV9.CAG.GFP, ICV | 24 | h | 0 | 0 |
| MR-18-13 | AAV9.CAG.GFP, ICV | 24 | h | + | 0 |
| MR-18-14 | AAV9.CAG.GFP, ICV | 24 | h | + | 0 |
| MR-18-15 | AAV9.CAG.GFP, ICV | 24 | h | 0 | 0 |
| MR-18-16 | AAV9.CAG.GFP, ICV | 24 | h | 0 | 0 |

| | | | | |
|---|---|---|---|---|
| MR-18-17 | AAV9.CAG.GFP, IV | — | 0 | 0 |
| MR-18-18 | AAV9.CAG.GFP, IV | — | 0 | 0 |
| MR-18-19 | AAV9.CAG.GFP, IV | — | 0 | 0 |

*MR-18-7 to MR-18-16 had positive NAb responses and MR-18-2 to MR-18-6 and MR-18-17 to MR-18-19 had negative NAb responses.

TABLE 9
Percentage of GFP⁺ Lumbar DRG

| | | | | GFP % + | |
|---|---|---|---|---|---|
| Animal # | Vector Treatment | IVIG* | | Cells | −Control |
| MR-18-1 | Saline | — | | 0 | 0 |
| MR-18-2 | AAV9.CAG.GFP, ICV | — | | 0 | 0 |
| MR-18-3 | AAV9.CAG.GFP, ICV | — | | + | 0 |
| MR-18-4 | AAV9.CAG.GFP, ICV | — | | 0 | 0 |
| MR-18-5 | AAV9.CAG.GFP, ICV | — | | 0 | 0 |
| MR-18-6 | AAV9.CAG.GFP, ICV | — | | 0 | 0 |
| MR-18-7 | AAV9.CAG.GFP, ICV | 2 | h | 0 | 0 |
| MR-18-8 | AAV9.CAG.GFP, ICV | 2 | h | + | 0 |
| MR-18-9 | AAV9.CAG.GFP, ICV | 2 | h | + | 0 |
| MR-18-10 | AAV9.CAG.GFP, ICV | 2 | h | + | 0 |
| MR-18-11 | AAV9.CAG.GFP, ICV | 2 | h | + | 0 |
| MR-18-12 | AAV9.CAG.GFP, ICV | 24 | h | Equivocal+ | 0 |
| MR-18-13 | AAV9.CAG.GFP, ICV | 24 | h | + | 0 |
| MR-18-14 | AAV9.CAG.GFP, ICV | 24 | h | + | 0 |
| MR-18-15 | AAV9.CAG.GFP, ICV | 24 | h | + | 0 |
| MR-18-16 | AAV9.CAG.GFP, ICV | 24 | h | 0 | 0 |

| | | | | |
|---|---|---|---|---|
| MR-18-17 | AAV9.CAG.GFP, IV | — | 0 | 0 |
| MR-18-18 | AAV9.CAG.GFP, IV | — | 0 | 0 |
| MR-18-19 | AAV9.CAG.GFP, IV | — | 0 | 0 |

*MR-18-7 to MR-18-16 had positive NAb responses and MR-18-2 to MR-18-6 and MR-18-17 to MR-18-19 had negative NAb responses.

FIG. 3F shows representative images of heart cross-sections obtained after anti-GFP IHC. GFP expression was observed in the heart in groups treated with AAV9.CAG.GFP by either ICV or IV administration. In animals administered IVIG either 2 hours or 24 hours prior to AAV9.CAG.GFP ICV administration, no GFP expression was detected in the heart, which was similar to saline injected control. Table 10 provides a summary of percentage of GFP⁺ heart cells for each animal.

TABLE 10
Percentage of GFP⁺ Heart Cells

| | | | | GFP % + | |
|---|---|---|---|---|---|
| Animal # | Vector Treatment | IVIG* | | Cells | −Control |
| MR-18-1 | Saline | — | | 0 | 0 |
| MR-18-2 | AAV9.CAG.GFP, ICV | — | | 2 | 0 |
| MR-18-3 | AAV9.CAG.GFP, ICV | — | | 0+ | 0 |
| MR-18-4 | AAV9.CAG.GFP, ICV | — | | 1 | 0 |
| MR-18-5 | AAV9.CAG.GFP, ICV | — | | 0+ | 0 |
| MR-18-6 | AAV9.CAG.GFP, ICV | — | | 1 | 0 |
| MR-18-7 | AAV9.CAG.GFP, ICV | 2 | h | 0 | 0 |
| MR-18-8 | AAV9.CAG.GFP, ICV | 2 | h | 0 | 0 |
| MR-18-9 | AAV9.CAG.GFP, ICV | 2 | h | 0 | 0 |
| MR-18-10 | AAV9.CAG.GFP, ICV | 2 | h | 0 | 0 |
| MR-18-11 | AAV9.CAG.GFP, ICV | 2 | h | 0 | 0 |
| MR-18-12 | AAV9.CAG.GFP, ICV | 24 | h | 0 | 0 |
| MR-18-13 | AAV9.CAG.GFP, ICV | 24 | h | 0 | 0 |
| MR-18-14 | AAV9.CAG.GFP, ICV | 24 | h | 0 | 0 |
| MR-18-15 | AAV9.CAG.GFP, ICV | 24 | h | 0 | 0 |
| MR-18-16 | AAV9.CAG.GFP, ICV | 24 | h | 0 | 0 |

| | | | | |
|---|---|---|---|---|
| MR-18-17 | AAV9.CAG.GFP, IV | — | 2 | 0 |
| MR-18-18 | AAV9.CAG.GFP, IV | — | 2 | 0 |
| MR-18-19 | AAV9.CAG.GFP, IV | — | 2 | 0 |

*MR-18-7 to MR-18-16 had positive NAb responses and MR-18-2 to MR-18-6 and MR-18-17 to MR-18-19 had negative NAb responses.

FIG. 3G shows representative images of kidney cross-sections obtained after anti-GFP IHC. GFP expression was observed in the kidney in groups treated with AAV9.CAG.GFP by either ICV or IV administration. GFP expression was limited to tubules adjacent to glomeruli, suggesting expression specifically in epithelial cells representing the macula densa. In animals administered IVIG either 2 hours or 24 hours prior to AAV9.CAG.GFP ICV administration, no GFP expression was detected in the kidney, which was similar to saline injected control. Table 11 provides a summary of percentage of GFP⁺ kidney cells for each animal.

TABLE 11
Percentage of GFP⁺ Kidney Cells

| | | | | GFP % + | |
|---|---|---|---|---|---|
| Animal # | Vector Treatment | IVIG* | | Cells | −Control |
| MR-18-1 | Saline | — | | 0 | 0 |
| MR-18-2 | AAV9.CAG.GFP, ICV | — | | 0+ | 0 |
| MR-18-3 | AAV9.CAG.GFP, ICV | — | | 0 | 0 |
| MR-18-4 | AAV9.CAG.GFP, ICV | — | | 0+ | 0 |
| MR-18-5 | AAV9.CAG.GFP, ICV | — | | 0+ | 0 |
| MR-18-6 | AAV9.CAG.GFP, ICV | — | | 0+ | 0 |
| MR-18-7 | AAV9.CAG.GFP, ICV | 2 | h | 0 | 0 |
| MR-18-8 | AAV9.CAG.GFP, ICV | 2 | h | 0 | 0 |
| MR-18-9 | AAV9.CAG.GFP, ICV | 2 | h | 0 | 0 |
| MR-18-10 | AAV9.CAG.GFP, ICV | 2 | h | 0 | 0 |
| MR-18-11 | AAV9.CAG.GFP, ICV | 2 | h | 0 | 0 |
| MR-18-12 | AAV9.CAG.GFP, ICV | 24 | h | 0 | 0 |
| MR-18-13 | AAV9.CAG.GFP, ICV | 24 | h | 0 | 0 |
| MR-18-14 | AAV9.CAG.GFP, ICV | 24 | h | 0 | 0 |
| MR-18-15 | AAV9.CAG.GFP, ICV | 24 | h | 0 | 0 |
| MR-18-16 | AAV9.CAG.GFP, ICV | 24 | h | 0 | 0 |

| | | | | |
|---|---|---|---|---|
| MR-18-17 | AAV9.CAG.GFP, IV | — | 0+ | 0 |
| MR-18-18 | AAV9.CAG.GFP, IV | — | 0+ | 0 |
| MR-18-19 | AAV9.CAG.GFP, IV | — | 0+ | 0 |

*MR-18-7 to MR-18-16 had positive NAb responses and MR-18-2 to MR-18-6 and MR-18-17 to MR-18-19 had negative NAb responses.

6.5.1.7. Conclusion

This experiment was designed to test the hypothesis that systemic IVIG administration prior to ICV administration of AAV9.CAG.GFP limits transduction of peripheral organs by AAV9.CAG.GFP that has escaped the CNS.

Vector genomes were observed in the liver, heart, and kidney in groups treated with AAV9.CAG.GFP by either ICV or IV administration. The presence of vector genomes in peripheral organs suggests that AAV is adept at escaping the CNS after ICV injection. Compared to animals that were only dosed with ICV, animals that were treated with IVIG prior to ICV dosing of AAV showed significantly reduced transduction and expression in the liver, heart, and kidney. While the spleen showed an increase in vector genomes in animals pre-treated with IVIG, this is likely due to engulfment of antibody-bound capsid by circulating phagocytic cells rather than bona fide transduction. In support of this, GFP expression data (IHC or RT-ddPCR) showed no significant change between groups administered IVIG and those that were dosed with AAV without IVIG administration. Overall, administration of IVIG prior to AAV ICV administration limited AAV transduction in peripheral tissues, including liver, heart, and kidney.

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

SEQUENCE APPENDIX

| | For Anc library sequences (Anc80; Anc81; Anc82; Anc83; |
|---|---|
| | Anc84; Anc94; Anc110; Anc113; Anc126; and Anc127), X |
| SEQ ID | can be any one of the amino acids listed below for |
| NO | each toggle site site |
| SEQ ID | MAADGYLPDWLEDNLSEGIREWWDLKPGAPKPKANQQKQDDGRGLVLPGYKYLGPFNGLDK |
| NO: 54 | GEPVNAADAAALEHDKAYDQQLKAGDNPYLRYNHADAEFQERLQEDTSFGGNLGRAVFQAKK |
| (AAV1 | RVLEPLGLVEEGAKTAPGKKRPVEQSPQEPDSSSGIGKTGQQPAKKRLNFGQTGDSESVPDPQPL |
| (AAD277 | GEPPATPAAVGPTTMASGGGAPMADNNEGADGVGNASGNWHCDSTWLGDRVITTSTRTWA |
| 57)) | LPTYNNHLYKQISSASTGASNDNHYFGYSTPWGYFDFNRFHCHFSPRDWQRLINNNWGFRPKR |
| | LNFKLFNIQVKEVTTNDGVTTIANNLTSTVQVFSDSEYQLPYVLGSAHQGCLPPFPADVFMIPQY |
| | GYLTLNNGSQAVGRSSFYCLEYFPSQMLRTGNNFTFSYTFEEVPFHSSYAHSQSLDRLMNPLIDQ |
| | YLYYLNRTQNQSGSAQNKDLLFSRGSPAGMSVQPKNWLPGPCYRQQRVSKTKTDNNNSNFTW |
| | TGASKYNLNGRESIINPGTAMASHKDDEDKFFPMSGVMIFGKESAGASNTALDNVMITDEEEIK |
| | ATNPVATERFGTVAVNFQSSSTDPATGDVHAMGALPGMVWQDRDVYLQGPIWAKIPHTDGH |
| | FHPSPLMGGFGLKNPPPQILIKNTPVPANPPAEFSATKFASFITQYSTGQVSVEIEWELQKENSKR |
| | WNPEVQYTSNYAKSANVDFTVDNNGLYTEPRPIGTRYLTRPL |
| SEQ ID | MAADGYLPDWLEDTLSEGIRQWWKLKPGPPPPKPAERHKDDSRGLVLPGYKYLGPFNGLDKGE |
| NO: 55 | PVNEADAAALEHDKAYDRQLDSGDNPYLKYNHADAEFQERLKEDTSFGGNLGRAVFQAKKRVL |
| (AAV2 | EPLGLVEEPVKTAPGKKRPVEHSPVEPDSSSGTGKAGQQPARKRLNFGQTGDADSVPDPQPLG |
| (AAC037 | QPPAAPSGLGTNTMATGSGAPMADNNEGADGVGNSSGNWHCDSTWMGDRVITTSTRTWAL |
| 80)) | PTYNNHLYKQISSQSGASNDNHYFGYSTPWGYFDFNRFHCHFSPRDWQRLINNNWGFRPKRLN |
| | FKLFNIQVKEVTQNDGTTTIANNLTSTVQVFTDSEYQLPYVLGSAHQGCLPPFPADVFMVPQYGY |
| | LTLNNGSQAVGRSSFYCLEYFPSQMLRTGNNFTFSYTFEDVPFHSSYAHSQSLDRLMNPLIDQYLY |
| | YLSRTNTPSGTTTQSRLQFSQAGASDIRDQSRNWLPGPCYRQQRVSKTSADNNNSEYSWTGAT |
| | KYHLNGRDSLVNPGPAMASHKDDEEKFFPQSGVLIFGKQGSEKTNVDIEKVMITDEEEIRTTNPV |
| | ATEQYGSVSTNLQRGNRQAATADVNTQGVLPGMVWQDRDVYLQGPIWAKIPHTDGHFHPSP |
| | LMGGFGLKHPPPQILIKNTPVPANPSTTFSAAKFASFITQYSTGQVSVEIEWELQKENSKRWNPEI |
| | QYTSNYNKSVNVDFTVDTNGVYSEPRPIGTRYLTRNL |
| SEQ ID | MAADGYLPDWLEDNLSEGIREWWALKPGVPQPKANQQHQDNRRGLVLPGYKYLGPGNGLDK |
| NO: 56 | GEPVNEADAAALEHDKAYDQQLKAGDNPYLKYNHADAEFQERLQEDTSFGGNLGRAVFQAKK |
| (AAV3 | RILEPLGLVEEAAKTAPGKKGAVDQSPQEPDSSSGVGKSGKQPARKRLNFGQTGDSESVPDPQPL |
| (AAC550 | GEPPAAPTSLGSNTMASGGGAPMADNNEGADGVGNSSGNWHCDSQWLGDRVITTSTRTWA |
| 49)) | LPTYNNHLYKQISSQSGASNDNHYFGYSTPWGYFDFNRFHCHFSPRDWQRLINNNWGFRPKKL |
| | SFKLFNIQVRGVTQNDGTTTIANNLTSTVQVFTDSEYQLPYVLGSAHQGCLPPFPADVFMVPQY |
| | GYLTLNNGSQAVGRSSFYCLEYFPSQMLRTGNNFQFSYTFEDVPFHSSYAHSQSLDRLMNPLIDQ |
| | YLYYLNRTQGTTSGTTNQSRLLFSQAGPQSMSLQARNWLPGPCYRQQRLSKTANDNNNSNFP |
| | WTAASKYHLNGRDSLVNPGPAMASHKDDEEKFFPMHGNLIFGKEGTTASNAELDNVMITDEEE |
| | IRTTNPVATEQYGTVANNLQSSNTAPTTGTVNHQGALPGMVWQDRDVYLQGPIWAKIPHTDG |
| | HFHPSPLMGGFGLKHPPPQIMIKNTPVPANPPTTFSPAKFASFITQYSTGQVSVEIEWELQKENS |
| | KRWNPEIQYTSNYNKSVNVDFTVDTNGVYSEPRPIGTRYLTRNL |
| SEQ ID | MSFVDHPPDWLEEVGEGLREFLGLEAGPPKPKPNQQHQDQARGLVLPGYNYLGPGNGLDRGE |
| NO: 57 | PVNRADEVAREHDISYNEQLEAGDNPYLKYNHADAEFQEKLADDTSFGGNLGKAVFQAKKRVLE |
| (AAV5 | PFGLVEEGAKTAPTGKRIDDHFPKRKKARTEEDSKPSTSSDAEAGPSGSQQLQIPAQPASSLGADT |
| (AAD137 | MSAGGGGPLGDNNQGADGVGNASGDWHCDSTWMGDRVVTKSTRTWVLPSYNNHQYREIK |
| 56)) | SGSVDGSNANAYFGYSTPWGYFDFNRFHSHWSPRDWQRLINNYWGFRPRSLRVKIFNIQVKEV |
| | TVQDSTTTIANNLTSTVQVFTDDDYQLPYVVGNGTEGCLPAFPPQVFTLPQYGYATLNRDNTEN |
| | PTERSSFFCLEYFPSKMLRTGNNFEFTYNFEEVPFHSSFAPSQNLFKLANPLVDQYLYRFVSTNNT |
| | GGVQFNKNLAGRYANTYKNWFPGPMGRTQGWNLGSGVNRASVSAFATTNRMELEGASYQV |
| | PPQPNGMTNNLQGSNTYALENTMIFNSQPANPGTTATYLEGNMLITSESETQPVNRVAYNVGG |
| | QMATNNQSSTTAPATGTYNLQEIVPGSVWMERDVYLQGPIWAKIPETGAHFHPSPAMGGFGL |
| | KHPPPMMLIKNTPVPGNITSFSDVPVSSFITQYSTGQVTVEMEWELKKENSKRWNPEIQYTNNY |
| | NDPQFVDFAPDSTGEYRTTRPIGTRYLTRPL |
| SEQ ID | MAADGYLPDWLEDNLSEGIREWWDLKPGAPKPKANQQKQDDGRGLVLPGYKYLGPFNGLDK |
| NO: 58 | GEPVNAADAAALEHDKAYDQQLKAGDNPYLRYNHADAEFQERLQEDTSFGGNLGRAVFQAKK |
| (AAV6 | RVLEPFGLVEEGAKTAPGKKRPVEQSPQEPDSSSGIGKTGQQPAKKRLNFGQTGDSESVPDPQPL |
| (AAB954 | GEPPATPAAVGPTTMASGGGAPMADNNEGADGVGNASGNWHCDSTWLGDRVITTSTRTWA |
| 50)) | LPTYNNHLYKQISSASTGASNDNHYFGYSTPWGYFDFNRFHCHFSPRDWQRLINNNWGFRPKR |
| | LNFKLFNIQVKEVTTNDGVTTIANNLTSTVQVFSDSEYQLPYVLGSAHQGCLPPFPADVFMIPQY |
| | GYLTLNNGSQAVGRSSFYCLEYFPSQMLRTGNNFTFSYTFEDVPFHSSYAHSQSLDRLMNPLIDQ |
| | YLYYLNRTQNQSGSAQNKDLLFSRGSPAGMSVQPKNWLPGPCYRQQRVSKTKTDNNNSNFTW |
| | TGASKYNLNGRESIINPGTAMASHKDDKDKFFPMSGVMIFGKESAGASNTALDNVMITDEEEIK |
| | ATNPVATERFGTVAVNLQSSSTDPATGDVHVMGALPGMVWQDRDVYLQGPIWAKIPHTDGH |
| | FHPSPLMGGFGLKHPPPQILIKNTPVPANPPAEFSATKFASFITQYSTGQVSVEIEWELQKENSKR |
| | WNPEVQYTSNYAKSANVDFTVDNNGLYTEPRPIGTRYLTRPL |
| SEQ ID | MAADGYLPDWLEDNLSEGIREWWDLKPGAPKPKANQQKQDNGRGLVLPGYKYLGPFNGLDK |
| NO: 59 | GEPVNAADAAALEHDKAYDQQLKAGDNPYLRYNHADAEFQERLQEDTSFGGNLGRAVFQAKK |
| (AAV7 | RVLEPLGLVEEGAKTAPAKKRPVEPSPQRSPDSSTGIGKKGQQPARKRLNFGQTGDSESVPDPQP |
| (AF51385 | LGEPPAAPSSVGSGTVAAGGGAPMADNNEGADGVGNASGNWHCDSTWLGDRVITTSTRTW |
| 1_2)) | ALPTYNNHLYKQISSETAGSTNDNTYFGYSTPWGYFDFNRFHCHFSPRDWQRLINNNWGFRPK |
| | KLRFKLFNIQVKEVTTNDGVTTIANNLTSTIQVFSDSEYQLPYVLGSAHQGCLPPFPADVFMIPQY |
| | GYLTLNNGSQSVGRSSFYCLEYFPSQMLRTGNNFEFSYSFEDVPFHSSYAHSQSLDRLMNPLIDQ |
| | YLYYLARTQSNPGGTAGNRELQFYQGGPSTMAEQAKNWLPGPCFRQQRVSKTLDQNNNSNFA |
| | WTGATKYHLNGRNSLVNPGVAMATHKDDEDRFFPSSGVLIFGKTGATNKTTLENVLMTNEEEIR |
| | PTNPVATEEYGIVSSNLQAANTAAQTQVVNNQGALPGMVWQNRDVYLQGPIWAKIPHTDGN |
| | FHPSPLMGGFGLKHPPPQILIKNTPVPANPPEVFTPAKFASFITQYSTGQVSVEIEWELQKENSKR |
| | WNPEIQYTSNFEKQTGVDFAVDSQGVYSEPRPIGTRYLTRNL |
| SEQ ID | MAADGYLPDWLEDNLSEGIREWWALKPGAPKPKANQQKQDDGRGLVLPGYKYLGPFNGLDK |
| NO: 60 | GEPVNAADAAALEHDKAYDQQLQAGDNPYLRYNHADAEFQERLQEDTSFGGNLGRAVFQAKK |
| (AAV8 | RVLEPLGLVEEGAKTAPGKKRPVEPSPQRSPDSSTGIGKKGQQPARKRLNFGQTGDSESVPDPQP |
| (AF51385 | LGEPPAAPSGVGPNTMAAGGGAPMADNNEGADGVGSSSGNWHCDSTWLGDRVITTSTRTW |
| 2_2)) | ALPTYNNHLYKQISNGTSGGATNDNTYFGYSTPWGYFDFNRFHCHFSPRDWQRLINNNWGFRP |
| | KRLSFKLFNIQVKEVTQNEGTKTIANNLTSTIQVFTDSEYQLPYVLGSAHQGCLPPFPADVFMIPQ |
| | YGYLTLNNGSQAVGRSSFYCLEYFPSQMLRTGNNFQFTYTFEDVPFHSSYAHSQSLDRLMNPLID |
| | QYLYYLSRTQTTGGTANTQTLGFSQGGPNTMANQAKNWLPGPCYRQQRVSTTTGQNNNSNF |
| | AWTAGTKYHLNGRNSLANPGIAMATHKDDEERFFPSNGILIFGKQNAARDNADYSDVMLTSEE |
| | EIKTTNPVATEEYGIVADNLQQQNTAPQIGTVNSQGALPGMVWQNRDVYLQGPIWAKIPHTD |
| | GNFHPSPLMGGFGLKHPPPQILIKNTPVPADPPTTFNQSKLNSFITQYSTGQVSVEIEWELQKENS |
| | KRWNPEIQYTSNYYKSTSVDFAVNTEGVYSEPRPIGTRYLTRNL |
| SEQ ID | MAADGYLPDWLEDNLSEGIREWWALKPGAPQPKANQQHQDNARGLVLPGYKYLGPGNGLDK |
| NO: 61 | GEPVNAADAAALEHDKAYDQQLKAGDNPYLKYNHADAEFQERLKEDTSFGGNLGRAVFQAKKR |
| (AAV9 | LLEPLGLVEEAAKTAPGKKRPVEQSPQEPDSSAGIGKSGAQPAKKRLNFGQTGDTESVPDPQPIG |
| (AAS9926 | EPPAAPSGVGSLTMASGGGAPVADNNEGADGVGSSSGNWHCDSQWLGDRVITTSTRTWALP |
| 4)) | TYNNHLYKQISNSTSGGSSNDNAYFGYSTPWGYFDFNRFHCHFSPRDWQRLINNNWGFRPKRL |
| | NFKLFNIQVKEVTDNNGVKTIANNLTSTVQVFTDSDYQLPYVLGSAHEGCLPPFPADVFMIPQYG |
| | YLTLNDGSQAVGRSSFYCLEYFPSQMLRTGNNFQFSYEFENVPFHSSYAHSQSLDRLMNPLIDQY |
| | LYYLSKTINGSGQNQQTLKFSVAGPSNMAVQGRNYIPGPSYRQQRVSTTVTQNNNSEFAWPGA |
| | SSWALNGRNSLMNPGPAMASHKEGEDRFFPLSGSLIFGKQGTGRDNVDADKVMITNEEEIKTT |
| | NPVATESYGQVATNHQSAQAQAQTGWVQNQGILPGMVWQDRDVYLQGPIWAKIPHTDGNF |
| | HPSPLMGGFGMKHPPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 62 | GEPVNAADAAALEHDKAYDQQLKAGDNPYLRYNHADAEFQERLQEDTSFGGNLGRAVFQAKK |
| (AAV10 | RVLEPLGLVEEAAKTAPGKKRPVEPSPQRSPDSSTGIGKKGQQPAKKRLNFGQTGESESVPDPQPI |
| (AAT4633 | GEPPAGPSGLGSGTMAAGGGAPMADNNEGADGVGSSSGNWHCDSTWLGDRVITTSTRTWA |
| 7)) | LPTYNNHLYKQISNGTSGGSTNDNTYFGYSTPWGYFDFNRFHCHFSPRDWQRLINNNWGFRPK |
| | RLSFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFEFSYTFEDVPFHSSYAHSQSLDRLMNPLIDQ |
| | YLYYLSRTQSTGGTQGTQQLLFSQAGPANMSAQAKNWLPGPCYRQQRVSTTLSQNNNSNFAW |
| | TGATKYHLNGRDSLVNPGVAMATHKDDEERFFPSSGVLMFGKQGAGRDNVDYSSVMLTSEEEI |
| | KTTNPVATEQYGVVADNLQQANTGPIVGNVNSQGALPGMVWQNRDVYLQGPIWAKIPHTDG |
| | NFHPSPLMGGFGLKHPPPQILIKNTPVPADPPTTFSQAKLASFITQYSTGQVSVEIEWELQKENSK |
| | RWNPEIQYTSNYYKSTNVDFAVNTEGTYSEPRPIGTRYLTRNL |
| SEQ ID | MAADGYLPDWLEDNLSEGIREWWALKPGAPQPKANQQHQDNARGLVLPGYKYLGPGNGLDK |
| NO: 63 | GEPVNEADAAALEHDKAYDQQLKAGDNPYLKYNHADAEFQERLKEDTSFGGNLGRAVFQAKKR |
| (AAV | LLEPLGLVEEAAKTAPGKKRPVEQSPQEPDSSVGIGKSGAQPAKKRLNFGQTGDTESVPDPQPIG |
| hu.68) | EPPAAPSGVGSLTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQRLINNNWGFRPKRL |
| | NFKLFNIQVKEVTDNNGVKTIANNLTSTVQVFTDSDYQLPYVLGSAHEGCLPPFPADVFMIPQYG |
| | YLTLNDGSQAVGRSSFYCLEYFPSQMLRTGNNFQFSYEFENVPFHSSYAHSQSLDRLMNPLIDQY |
| | LYYLSKTINGSGQNQQTLKFSVAGPSNMAVQGRNYIPGPSYRQQRVSTTVTQNNNSEFAWPGA |
| | SSWALNGRNSLMNPGPAMASHKEGEDRFFPLSGSLIFGKQGTGRDNVDADKVMITNEEEIKTT |
| | NPVATESYGQVATNHQSAQAQAQTGWVQNQGILPGMVWQDRDVYLQGPIWAKIPHTDGNF |
| | HPSPLMGGFGMKHPPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL* |
| SEQ ID | MAADGYLPDWLEDNLSEGIREWWALQPGAPKPKANQQHQDNARGLVLPGYKYLGPGNGLDK |
| NO: 64 | GEPVNAADAAALEHDKAYDQQLKAGDNPYLKYNHADAEFQERLKEDTSFGGNLGRAVFQAKKR |
| (AAV | LLEPLGLVEEAAKTAPGKKRPVDQSPQEPDSSSGVGKSGKQPARKRLNFGQTGDSESVPDPQPL |
| LK03) | GEPPAAPTSLGSNTMASGGGAPMADNNEGADGVGNSSGNWHCDSQWLGDRVITTSTRTWA |
| | LPTYNNHLYKQISSQSGASNDNHYFGYSTPWGYFDFNRFHCHFSPRDWQRLINNNWGFRPKKL |
| | SFKLFNIQVKEVTQNDGTTTIANNLTSTVQVFTDSEYQLPYVLGSAHQGCLPPFPADVFMVPQYG |
| | YLTLNNGSQAVGRSSFYCLEYFPSQMLRTGNNFQFSYTFEDVPFHSSYAHSQSLDRLMNPLIDQY |
| | LYYLNRTQGTTSGTTNQSRLLFSQAGPQSMSLQARNWLPGPCYRQQRLSKTANDNNNSNFPW |
| | TAASKYHLNGRDSLVNPGPAMASHKDDEEKFFPMHGNLIFGKEGTTASNAELDNVMITDEEEIR |
| | TTNPVATEQYGTVANNLQSSNTAPTTRTVNDQGALPGMVWQDRDVYLQGPIWAKIPHTDGHF |
| | HPSPLMGGFGLKHPPPQIMIKNTPVPANPPTTFSPAKFASFITQYSTGQVSVEIEWELQKENSKR |
| | WNPEIQYTSNYNKSVNVDFTVDTNGVYSEPRPIGTRYLTRPL* |
| SEQ ID | MAADGYLPDWLEDTLSEGIRQWWKLKPGPPPPKPAERHKDDSRGLVLPGYKYLGPFNGLDKGE |
| NO: 65 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPVEPDSSSGTGKAGQQPARKRLNFGQTGDADSVPDPQPLG |
| hu.1 | QPPAAPSGLGSTTMATGSGAPMADNNEGADGVGNSSGNWHCDSQWLGDRVITTSTRTWAL |
| (AAS9926 | PTYNNHLYKQISSQSGASNDNHYFGYSTPWGYFDFNRFHCHFSPRDWQRLINNNWGFRPKRLN |
| 0)) | FKLFNIQVKEVTQNGGTTTIANNLTSTVQVFTDSEYQLPYVLGSAHQGCLPPFPADVFMVPQYGY |
| | LTLNNGSQAVGRSSFYCLEYFPSQMLRTGNNFQFSYTFEDVPFHSSYAHSQSLDRLMNPLIDQYL |
| | YYLNKTQTNSGTLQQSRLLFSQAGPTNMSLQAKNWLPGPCYRQQRLSKQANGNNNSNFPWTA |
| | ATKYHLNGRDSLVNPGPAMASHKDDEEKFFPMHGTLIFGKQGTNANDADLENVMITDEEEIRA |
| | TNPVATEQYGTVSNNLQNSNTGPTTGTVNHQGALPGMVWQDRDVYLQGPIWAKIPHTDGHF |
| | HPSPLTGGFGLKHPPPQIMIKNTPVPANPPTNFSSAKFASFITQYSTGQVSVEIEWELQKENSKR |
| | WNPEIQYTSNYNKSVNVDFTVDTNGVYSEPRPIGTRYLTRNL |
| SEQ ID | MAADGYPPDWLEDTLSEGIRQWWKLKPGPPPPKPAERHKDDSRGLVLPGYKYLGPFNGLDKGE |
| NO: 66 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPVEPDSSSGTGKAGQRPARKRLNFGQTGDADSVPDPQPLGQ |
| hu.2 | PPAAPSGLGSTTMATGSGAPMADNNEGADGVGNSSGNWHCDSQWLGDRVITTSTRTWALPT |
| (AAS9927 | YNNHLYKQISSQSGASNDNHYFGYSTPWGYFDFNRFHCHFSPRDWQRLINNNWGFRPKRLNFK |
| 0)) | LFNIQVKEVTQNDGTTTIANNLTSTVQVFTDSEYQLPYVLGSAHQGCLPPFPADVFMVPQYGYLT |
| | LNNGSQAVGRSSFYCLEYFPSQMLRTGNNFQFSYTFEDVPFHSSYAHSQSLDRLMNPLIDQYLYY |
| | LNKTQTNSGTLQQSRLLFSQAGPTNMSLQAKNWLPGPCYRQQRLSKQANDNNNSNFPWTAA |
| | TKYHLNGRDSLVNPGPAMASHKDDEEKFFPMHGTLIFGKQGTNANDADLENVMITDEEEIRAT |
| | NPVATEQYGTVSNNLQNSNTGPTTGTVNRQGALPGMVWQDRDVYLQGPIWAKIPHTDGHFH |
| | PSPLMGGFGLKHPPPQIMIKNTPVPANPPTNFSSAKFASFITQYSTGQVSVEIEWELQKENSKRW |
| | NPEIQYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 67 | PVNEADAAALEHDKAYDRQLDSGDNPYLKYNHADAEFQERLKEDTSFGGNLGRAVFQAKKRVL |
| (AAV | EPLRPGLRKPVKTAPGKKRPVEHSPVEPDSSSGTGKAGQQPARKRLNFGQTGDADSVPDPQPLG |
| hu.3 | QPPAAPSGLGSTTMATGSGAPMADNNEGADGVGNSSGNWHCDSQWLDDRVIATSTRTWAL |
| (AAS9928 | PTYNNHLYKQISSQSGACNDNHYFGYSTPWGYFDFNRFHCHFSPRDWQRLINSNWGFRPKRLN |
| 0)) | FKLFNIQVKEVTQNDGTTTIANNLTSTVQVFTDSEYQLPYVPGSAHQGCLPPFPADVFMVPQYG |
| | YLTLNNGSQAVGRSSFYCLEYFPSQMLRTGNNFQFSYTFEDVPFHSSYAHCQSLDRLMNPLIDQY |
| | LYYLNKTQTNSGTLQQSRLLFSQAGPTNMSLQAKNWLPGPCYRQQRLSKQANDNNNCNFPWT |
| | AATKYHLNGRDSLVNPGPAMASHKDDEEKFFPMHGTLIFGKQGTNANDADLENVMITDEEEIR |
| | PTNPVATEQYGTVSNNLQNSNTGPTTGTVNHQGALPGMVWQDRDVYLQGPIWAKIPHTDGH |
| | FHPSPLMGGFGLKHPPPQIMIKSTPVPANPPTNFSSAKFASSITQYSTGQVSVEIEWELQKENSKR |
| | WNPEIQYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 68 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPVEPDSSSGTGKAGQQPARKRLNFGQTGDADSVPDPQPLG |
| hu.4 | QPPAAPSGLGSTTMATGSGAPMADNNEGADGVGNSSGNWHCDSQWLGDRVITTSTRTWAL |
| (AAS9928 | PTYNNHLYKQISSQSGASNDNHYFGYSTPWGYFDFNRFHCHFSPRDWQRLVNNNRGFRPKRLN |
| 7)) | FKLFNIQVKEVTQNDGTTTIANNLTSTVQVFTDSEYQLPYVLGSAHQGCLPPFPADVFMVPQYGY |
| | LTLNNGSQAVGRSSFYCLEYFPSQMLRTGNNFQFSYTFEDVPFHSSYAHSQSLDRLMNPLIDQYL |
| | YYLNKTQTNSGTLQQSRLLFSQAGPTNMSLQAKNWLPGPCYRQQRLSKQANDNNNSNFPWTA |
| | ATKYHLNGRDSLVNPGPAMASHKDDEEKFFPMHGTLIFGKQGTNANDADLENVMITDEEEIRA |
| | TNPVATEQYGTVSNNLQNSNTGPTTGTVNHQGALPGMVWQDRDVYLQGPIWAKIPHTDGHF |
| | HPSPLMGGFGLKHPPPQIMIKNTPVPANPPTNFSSAKFASFITQYSTGQVSVEIEWELQKENSKR |
| | WNPEIQYTSNYNKSVNVDFTVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 69 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPVEPSPQRSPDSSTGIGKTGQQPAKKRLNFGQTGDSESVPDPQP |
| hu.6 | IGEPPAGPSGLGSGTMAAGGGAPMADNNEGADGVGSSSGNWHCDSAWLGDRVITTSTRPW |
| (AAS9930 | ALPTYNNHLYKQISNGTSGGSTNDNTYFGYSTPWGYFDFNRFHCHFSPRDWQRLINNNWGFRP |
| 6)) | KRLNFKLFNIQVKEVTQNEGTKTIANNLTSTIQVFTDSEYQLPYVLGSAHQGCPPPFPADVFMIPQ |
| | YGYLTLNNGSQAVGRSSFYCLEYFPSQMRRTGNNFEFSYQFEDVPFHSSYAHSQSLDRLMNPLID |
| | QYLYYLSRTQSTGGTAGTQQLLFSQAGPNNMSAQAKNWLPGPCYRQQRVSTTLSQNNNSNFA |
| | WTGATKYHLNGRDSLVNPGVAMATHKDDEERFFPSSGVLMFGKQGAGKDNVDYSSVMLTSEE |
| | EIKTTNPVATEQYGVVADNLQQQNAAPIVGAVNSQGALPGMVWQNRDVYLQGPIWAKIPHT |
| | DGNFHPSPLMGGFGLKHPPPQILIKNTPVPADPPTTFSQAKLASFITQYSTGQVSVEIEWELQKEN |
| | SKRWNPEIQYTSNYYKSTNVDFAVNTEGTYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 70 | PVNEADAAALEHDKAYDRQLDSGDNPYLKYNHADAEFQERLKEDTSFGGNLGRAVFQAKKRVL |
| (AAV | EPLGLVEGPVKTAPGKKRPVEHSPAEPDSSSGTGKAGQQPARKRLNFGQTGDADSVPDPQPLG |
| hu.7 | QPPAAPSGLGSTTMATGSGAPMADNNEGADGVGNSSGNWHCDSQWLGDRVITTSTRTWAL |
| (AAS9931 | PTYNNHLYKQISSQSGASNDNHYFGYSTPWGYFDFNRFHCHFSPRDWQRLINNNWGFRPKRLN |
| 3)) | FKLFNIQVKEVTQNDGTTTIANNLTSTVQVFTDSEYQLPYVLGSAHQGCLPPFPADVFMVPQYGY |
| | LTLNNGSQAVGRSSFYCLEYFPSQMLRTGNNFQFSYTFEDVPFHSSYAHSQSLDRLMNPLIDQYL |
| | YYLNKTQSNSGTLQQSRLLFSQAGPTSMSLQAKNWLPGPCYRQQRLSKQANDNNNSNFPWTA |
| | ATKYHLNGRDSLVNPGPAMASHKDDEEKFFPMHGTLIFGKQGTNANDADLDNVMITDEEEIRT |
| | TNPVATEQYGYVSNNLQNSNTGPTTGTVNHQGALPGMVWQDRDVYLQGPIWAKIPHTDGHF |
| | HPSPLMGGFGLKHPPPQIMIKNTPVPANPPTNFSSAKFASFITQYSTGQVSVEIEWELQKENSKR |
| | WNPEIQYTSNYNKSVNVDFTVDTNGVYSEPRPIGTRYLTRNL |
| SEQ ID | MAADGYLPDWLEDTLSEGIRQWWKLKPGPPPPKPAERHQDNSRGLVLPGYKYLGPSNGLDKGE |
| NO: 71 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPVEPDSSSGTGKAGHQPARKRLNFGQTGDADSVPDPQPLGQ |
| hu.9 | PPAAPTSLGSTTMATGSGAPMADNNEGADGVGNSSGNWHCDSQWLGDRVITTSTRTWALPT |
| (AAS9931 | YNNHLYKQISSQSGASNDNHYFGCSTPWGYFDFNRFHCHFSPRDWQRLINNNWGFRPKRLNFK |
| 4)) | LFNIQVKEVTQNDGTTTIANNLTSTVQVFTDSEYPLPYVLGSAHQGCLPPFPADVFMVPQYGYLT |
| | LNNGSQAVGRSSFYCLEYFPSQMLRTGNNFTFSYTFEDVPFHSSYAHSQSLDRLMNPLIDQYLYYL |
| | NRTQSNSGTLQQSRLLFSQAGPTSMSLQAKNWLPGPCYRQQRLSKQANDNNNSNFPWTAATK |
| | YHLNGRDSLVNPGPAMASHKDDEEKFFPMHGTLIFGKQGTNANDADLEHVMITDEEEIRTTNP |
| | VATEQYGNVSNNLQNSNTGPTTENVNHQGALPGMVWQDRDVYLQGPIWAKIPHTDGHFHPS |
| | PLMGGFGLKHPPPQIMIKNTPVPANPPTNFSSAKFASFITQYSTGQVSVEIEWELQKENSKRWN |
| | PEIQYTSNYNKSVNVDFTVDTNGVYSEPCPIGTRYLTRNL |
| SEQ ID | MAADGYLPDWLEDTLSEGIRQWWKLKPGPPPPKLAERHQDDSRGLVLPGYKYLGPFNGLDKGE |
| NO: 72 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPVEPDSSSGTGKAGHQPARKRLNFGQTGDADSVPDPQPLGQ |
| hu.10 | PPAAPTSLGSTTMATGSGAPMADNNEGADGVGNSSGNWHCDSQWLGDRVITTSTRTWALPT |
| (AAS9926 | YNNHLYKQISSQSGASNDNHYFGYSTPWGYFDFNRFHCHFSPRDWQRLINNNWGFRPKRLNFK |
| 1)) | LFNIQVKEVTQNDGTTTIANNLTSTVQVFTDSEYQLPYVLGSAHQGCLPPFPADVFTVPQYGYLTL |
| | NNGSQAVGRSSFYCLEYFPSQMLRTGNNLTFSYTFEDVPFHSSYAHSQSLDRLMNPLIDQYLYYL |
| | NRTQSNSGTLQQSRLLFSQAGPTSMSLQAKNWLPGPCYRQQRLSKQANDNNNSNFPWTAATK |
| | YHLNGRDSLVNPGPAMASHKDDEEKFFPMHGTLIFGKQGTNANDADLEHVMITDEEEIRTTNP |
| | VATEQYGNVSNNLQNSNTGPTTENVNHQGALPGMVWQDRDVYLQGPIWAKIPHTDGHFHPS |
| | PLMGGFGLKHPPPQIMIKNTPVPANPPTNYSSAKFASFITQYSTGQVSVEIEWELRKENSKRWNP |
| | EIQYTSNYNKSVNVDFTVDTNGVYSEPRPIGTRYLTRNL |
| SEQ ID | MAADGYLPDWLEDTLSEGIRQWWKLKPGPPPPKPAERHQDDSRGLVLPGYKYLGPFNGLDKGE |
| NO: 73 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPVEPDSSSGTGKAGHQPARKRLNFGQTGDADSVPDPQPLGQ |
| hu.11 | PPAAPTSLGSTTMATGSGAPMADNNEGADGVGNSSGNWHCDSQWLGDRVITTSTRTWALPT |
| (AAS9926 | YNNHLYKQISSQSGASNDNHYFGYSTPWGYFDFNRFHCHFSPRDWQRLINNNWGFRPKRLNFK |
| 2)) | LFNIQVKEVTQNDGTTTIANNLTSTVQVFTDSEYQLPYVLGSAHQGCLPPFPADVFMVPQYGYLT |
| | LNNGSQAVGRSSFYCLEYFPSQMLRTGNNFTFSYTFEDVPFHSSYAHSQSLDRLMNPLIDQYLYYL |
| | NRTQSNSGTLQQSRLLFSQAGPTSMSLQAKNWLPGPCYRQQRLSKQANDNNNSNFPWTAATK |
| | YRLNGRDSLVNPGPAMASHKDDEEKFFPMHGTLIFGKQGTNANDADLEHVMITDEEEIRTTNP |
| | VATEQYGNVSNNLQNSNTGPTTENVNHQGALPGMVWQDRDVYLQGPIWAKIPHTDGHFHPS |
| | PLMGGFGLKHPPPQIMIKNTPVPANPPTNFSSAKFASFITQYSTGQVSVEIEWELQKENSKRWN |
| | PEIQYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLYKGE |
| NO: 74 | PVDEADAAALEHDKAYDRQLDSGDNPYLKYNHADAEFQERLKEDTSFGGNLGRAVFQAKKRVL |
| (AAV | EPLGLVGEPVKTAPGKKRPVEHSPVEPDSSSGTGKAGNQPARKRLNFGQTGDADSVPDPQPLG |
| hu.15 | QPPAAPSGLGSTTMATGSGAPVADNNEGADGVGNSSGNWHCDSQWLGDRVITTSTRTWALP |
| (AAS9926 | TYNNHLYKQISSQSGASNDNHYFGYSTPWGYFDFNRFHCHFSPRDRQRLINNNWGFRPKRLNF |
| 5)) | KLFNIQVKEVTQNDGTTTIANNLTSTVQVFTDSGYQLPYVLGLAHQGCLPPFPADVFMVPQYGY |
| | LTLNNGSQAVGRSSFYCLEYFPSQMLRTGNNFQFSYTFEDVPFHSSYAHSQSLDRLMNPLIDQYL |
| | YYLNKTQSNSGTLQQSRLLFSQAGPTSMSLQAKNWLPGPCYRQQRLSKQANDNNNSNFPWTA |
| | ATKYHLNGRDSLVNPGPAMASHKDDEEKFFPMHGTLIFGKQGTNANDADLDNVMITDEEEIRT |
| | TNPVATEQYGYVSNNLQNSNTGPTTGTVNHQGALPGMVWQDRDVYLQGPIWAKIPHTDGHF |
| | HPSPLMGGFGLKHPPPQIMIKNTPVPANPPTNFSSAKFASFITQYSTGQVSVEIEWELQKEDSKR |
| | WNPEIQYTSNYNKPVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLYKGE |
| NO: 75 | PVNEADAAALEHDKAYDRQLDSGDNPYLKYNHAGAEFQERLKEDTSFGGNLGRAVFQAKKRVL |
| (AAV | EPLGLVEEPVKTAPGKKRPVEHSPVEPDSSSGTGKAGNQPARKRLNFGQTGDADSVPDPQPLGQ |
| hu.16 | PPAAPSGLGSTTMATGSGAPVADNNEGADGVGNSSGNWHCDSQWLGDRVITTSTRTWALPT |
| (AAS9926 | YNNHLYKQISSQSGASNDNHYFGYSTPWGYFDFNRFHCHFSPRDWQRLINNNWGFRPKRLNFK |
| 6)) | LFNIQVKEVTQNDGTTTIANNLTSTVQVFTDSEYQLPYVLGSAHQGCLPPFPADVFMVPQYGYLT |
| | LNNGSQAVGRSSFYCLEYFPSQMLRTGNNFQFSYTFEDVPFHSSYAHSQSLDRLMNPLIDQYLYY |
| | LNKTQSNSGTLQQSRLLFSQAGPTSMSLQAKNWLPGPCYRQQRLSKQANDNNNSNFPWTAAT |
| | KYHLNGRDSLVNPGPAMASHKDDEEKFFPMHGTLIFGKQGTNANDADLDNVMITDEEEIRTTN |
| | PVATEQYGYVSNNLQDSNTGPTTGTVNHQGALPGMVWQDRDVYLQGPIWAKIPHTDGHFHP |
| | SPLMGGFGLKHPPPQIMIKNTPVPANPPTNFSSAKFASFITQYSTGQVSVEIEWELQKENSKRWN |
| | PEIQYTSNYNKSVNVDFTVDTNGVYSEPRPIGTRYLTRNL |
| SEQ ID | MAADGYLPDWLEDNLSEGIREWWDLKPGAPKPKANQQKQDDGRGLVLPGCKYLGPFNGLDK |
| NO: 76 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPVEPSPQRSPDSSTGIGKTGQQPAKKRLNFGQTGDSESVPDPQP |
| hu.17 | IGEPPAGPSGLGSGTMAAGGGAPMADNNEGADGVGSSSGNWHCDSTWLGDRVITTSTRTWA |
| (AAS9926 | LPTYNNHLYKQISNGTSGGSTNDNTYFGYSTPWGYFDFNRFHCHFSPRDWQRLINNNWGFRPK |
| 7)) | RLNFKLFNIQVKEVTQNEGTKTIANNLTSTIQVFTDSEYQLPYVLGSAHQGCPPPFPADVFMIPQY |
| | GYLTLNNGSQAVGRSSFYCLEYFPSQMRRTGNNFEFSYQFEDVPFHSSYAHSQSLDRLMNPLIDQ |
| | YLYYLSRTQSTGGTAGTQQLLFSQAGPNNMSAQAKNWLPGPCYRQQRVSTTLSQNNNSNFAW |
| | TGATKYHLNGRDSLVNPGVAMATHKDDEERFFPSSGVLMFGKQGAGKDNVDYSSVMLTSEEEI |
| | KTTNPVATEQYGVVADNLQQQNAAPIVGAVNSQGALPGMVWQNRDVYLQGPIWAKIPHTDG |
| | NFHPSPLMGGFGLKHPPPQILIKNTPVPADPPTTFSQAKLASFITQYSTGQVSVEIEWELQKENSK |
| | RWNPEIQYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 77 | PVNEADAAALEHDKAYDRQLESGDNPYLKYNHADAEFQERLKEDTSFGGNLGRAVFQAKKRVLE |
| (AAV | PLGLVEEPVKTAPGKKRPVEHSPVEPDSSSGTGKAGQQPARKRLNFGQTGDADSVPDPQPLGQ |
| hu.18 | PPAAPSGLGSTTMASGSGAPVADNNEGADGVGNSSGNWHCDSQWLGDRVITTSTRTWALPT |
| (AAS9926 | YNNHLYKQISSQSGASNDNHYFGYSTPWGYFDFNRFHCHFSPRDWQRLINNSWGFRPKRLNFK |
| 8)) | LFNIQVKEVTQNDGTTTIANNLTSTVQVFTDSEYQLPYVLGSAHQGCLPPFPADVFMVPQYGYLT |
| | LNNGSQAVGRSSFYCLEYFPSQMLRTGNNFQFSYTFEDVPFHSSYAHSQSLDRLLNPLIDQYLYYL |
| | NKTQSNSGTLQQSRLLFSQAGPTSMSLQAKNWLPGPCYRQQRLSKQANDNNNSNFPWTAATK |
| | YHLNGRDSLVNPGPAMASHKDDEEKFFPMHGTLIFGKQGTNANDADLDNVMITDEEEIRTTNP |
| | VATEQYGYVSNNLQNSNTGPTTGTVNHQGALPGMVWQDRDVYLQGPIWAKIPHTDGHFHPS |
| | PLMGGFGLKHPPPQIMIKNTPVPANPPTNFSSSKFASFITQYSTGQVSVEIEWELQKENSKRWNP |
| | EIQYTSNYNKSVNVDFTVDTNGVYSEPRPIGTRYPTRNL |
| SEQ ID | MAADGYLPDWLEDTLSEGIRQWWKLKPGPPPPKPAERHKDDSRGLVLPGYRYLGPFNGLDKGE |
| NO: 78 | PVNEADAAALEHDKAYDRQLDSGDNPYLKYNHVDAEFQERLKEDTSFGGNLGRAVFQAKKRILE |
| (AAV | PLGLVEEPVKAAPGEKRPVEHSPAEPDSSSGTGKAGQQPARKRLNFGQTGDADSVPDPQPLGQ |
| hu.20 | PPAAPSGLGTNTMASGSGAPMADNNEGADGVGNSSGNWHCDSTWMGDRVITTSTRTWALP |
| (AAS9927 | TYNNHLYKQISSQSGASNDNHYFGYSTPWGHFDFNRFHCHFSPRDWQRLINNNWGFRPKRLSF |
| 1)) | KLFNIQVKEVTQNDGTTTIANNLTSTVQVFTDSEYQLPYVLGSAHQGCLPPFPADVFMVPQYGYL |
| | TLNNGSQAVGRSSFYCLEYFPSQMLRTGNNFTFSYTFEDVPFHSSYAHSQSLDRLMNPLIDQYLY |
| | YLSRTNTPSGTTTMSRLQFSQAGASDIRDQSRNWLPGPCYRQQRVSKTAADNNNSDYSWTGAT |
| | KYHLNGRDSLVNPGPAMASHKDDEEKYFPQSGVLIFGKQDSGKTNVDIEKVMITDEEEIRTTNPV |
| | ATEQYGSVSTNLQSGNTQAATSDVNTQGVLPGMVWQDRDVYLQGPIWAKIPHTDGHFHPSPP |
| | MGGFGLKHPPPQILIKNTPVPANPSTTFSAAKFASFITQYSTGQVSVEIEWELQKENSKRWNPEIQ |
| | YTSNYNKSVNVDFTVDTNGVYSEPRPIGARYLTRNL |
| SEQ ID | MAADGYLPDWLEDTLSEGIRQWWKLKPGPPPPKPAERHKDDSRGLVLPGYKYLGPFNGLDKGE |
| NO: 79 | PVNEADAAALEHDKAYDRQLDSGDNPYLKYNHADAEFQERLKEDTSFGGNLGRAVFQAKKRILE |
| (AAV | PLGLVEEPVKTAPGKKRPVEHSPAEPDSSSGTGKAGQQPARKRLNFGQTGDADSVPDPRPLGQP |
| hu.21 | PAAPSGLGTNTMASGSGAPMADNNEGADGVGNSSGNWHCDSTWMGDRVITTSTRTWALPT |
| (AAS9927 | YNNHLYKQISSQSGASNDNHYFGYSTPWGYFDFNRFHCHFSPRDWQRLINNNWGFRPKRLSFK |
| 2)) | LFNIQVKEVTQNDGTTTIANNLTSTVQVFTDSEYQLPYVLGSAHQGCLPPFPADVFMVPQYGYLT |
| | LNNGSQAVGRSSFYCLEYFPSQMLRTGNNFTFSYTFEDVPFHSSYAHSQSLDRLMNPLIDQYLYYL |
| | SRTNTPSGTTTMSRLQFSQAGASDIRDQSRNWLPGPCYRQQRVSKTAADNNNSDYSWTGATK |
| | YHLNGRDSLVNPGPAMASHKDDEEKYFPQSGVLIFGKQDSGKTNVDIEKVMITDEEEIRTTNPVA |
| | TEQYGSVSTNLQSGNTQAATSDVNTQGVLPGMVWQDRDVYLQGPIWAKIPHTDGHFHPSPL |
| | MGGFGLKHPPPQILIKNTPVPANPSTTFSAAKFASFITQYSTGQVSVEIEWELQKENSKRWNPEIQ |
| | YTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 80 | PVNEADAAALEHDKAYDRQLDSGDNPYLKYNHADAEFQERLKGDTSFGGNLGRAVFQAKKRILE |
| (AAV | PLGLVEEPVKTAPGKKRPVEHSPAEPDSSSGTGKAGQQPARKRLNFGQTGDADSVPDPQPLGQ |
| hu.22 | PPAAPSGLGTNTMASGSGAPMADNNEGADGVGNSSGNWHCDSTWMGGRVITTSTRTWALP |
| (AAS9927 | TYNNHLYKQISSQSGASNDNHYFGYSTPWGYFDFNRFHCHFSPRDWQRLINNNWGFRPKRLSF |
| 3)) | KLFNIQVKEVTQNDGTTTIANNLTSTVQVFTDSEYQLPYVLGSAHQGCLPPFPADVFMVPQYGYL |
| | TLNNGSQAVGRSSFYCLEYFPSQTLRTGNNFTFSYTFEDVPFHSSYAHSQSLDRLMNPLIDQYLYY |
| | LSRTNTPSGTTTMSRLQFSQAGASDIRDQSRNWLPGPCYRQQRVSKTAADNNNSDYSWTGAT |
| | KYHLNGRDSLVNPGPAMASHKDDEEKYFPQSGVLIFGKQDSGKTNVDIEKVMITDEEEIRTTNPV |
| | ATEQYGSVSTNLQSGNTQAATSDVNTQGVLPGMVWQDRDVYLQGPIWAKIPHTDGHFHPSPL |
| | MGGFGLKHPPPQILIKNTPVPANPSTTFSAAKFASFITQYSTGQVSVEIEWELQKENSKRWNPEIQ |
| | YTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 81 | PVNEADAAALEHDKAYDRQLDSGDNPYLKYNHADAEFQERLKEDTSFGGNLGRAVFQAKKRILE |
| (AAV | PLGLVEEPVKTAPGKKRPVEHSPAEPDSSSGTGKAGQQPARKRLNFGQTGDADSVPDPQPLGQ |
| hu.23 | PPAAPSGLGTNTMASGSGAPMADNNEGADGVGNSSGNWHCDSTWMGDRVITTSTRTWALP |
| (AAS9927 | TCNNHLYKQISSQSGASNDNHYFGYSTPWGYFDFNRFHCHFSPRDWQRLINNNWGFRPKRLSF |
| 4)) | KLFNIQVKEVTQNDGTTTIANNLTSTVQVFTDSEYQLPYVLGSAHQGCLPPFPADVFMVPQYGYL |
| | TLNNGSQAVGRSSFYCLEYFPSQMLRTGNNFTFSYTFEDVPFHSSYAHSQSLDRLMNPLIDQYLY |
| | YLSRTNTPSGTTTMSRLQFSQAGASDIRDQSRNWLPGPCYRQQRVSKTAADNNNSDYSWTGAT |
| | KYHLNGRDSLVNPGPAMASHKDDEEKYFPQSGVLIFGKQDSGKTNVDIEKVMITDEEEIRTTNPV |
| | ATEQYGSVSTYLQSGNTQAATSDVNTQGVLPGMVWQDRDVYLRGPIWAKIPHTDGHFHPSPL |
| | MGGFGLKHPPPQILIKNTPVPANPSTTFSAAKFASFITQYSTGQVSVEIEWELQKENSKRWNPEIQ |
| | YTSNYNKSVNVDFTVDTNGVYSEPRPIGTRYLTRNL |
| SEQ ID | MAADGYLPDWLEDTLSEGIRQWWKLKPGPPPPKPAERHKDGSRGLVLPGYKYLGPFNGLDKGE |
| NO: 82 | PVNEADAAALEHDKAYDRQLNSGDNPYLKYNHADAEFQERLKEDTSFGGNLGRAVFQAKKRVL |
| (AAV | EPLGLVEEPVKTAPGKKRPVEHSPAEPDSSSGTGKAGQQPARKRLNFGQTGDADSVPDPQPLG |
| hu.25 | QPPAAPSGLGSTTMATGSGAPMADNNEGADGVGNSSGNWHCDSQWLGDRVITTSTRTWAL |
| (AAS9927 | PTYNNHLYKQISSQSGASNDNHYFGYSTPWGYFDFNRFHCHFSPRDWQRLINNNWGFRPKRLN |
| 6)) | FKLFNIQVKEVTQNDGTTTIANNLTSTVQVFTDSEYQLPYVLGSAHQGCLPPFPADVFMVPQYGY |
| | LTLNNGSQAVGRSPFYCLEYFPSQMLRTGNNFQFSYTFEDVPFHSSYAHSQSLDRLMNPLIDQYL |
| | YYLNKTQTNSGTLQQSRLLFSQAGPTNMSLQAKNWLPGPCYRQQRLSKQANDNNNSNFPWTA |
| | ATKYHLNGRDSLVNPGPAMASHKDDEEKFFPMHGTLIFGKQGTNANDADLENVMITDEEEIRTT |
| | NPVATEQYGTVSNNLQNSNTGPTTGTVNHQGALPGMVWQDRDVYLQGPIWAKIPHTDGHFH |
| | PSPLMGGFGLKHPPPQIMIKNTPVPANPPTNFSSAKFASFITQYSTGQVSVEIEWELQKENSKRW |
| | NPEIQYTSNYNKSVNVDFTVDNNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 83 | PVNEADAAALEHDKAYDRQLDSGDNPYLKYNHADAEFQERLKEDTSFGGNLGRAVFQAKKRILE |
| (AAV | PLGLVEEPVKTAPGKKRPVEHSPAEPDSSSGTGKAGQQPARKRLNFGQTGDADSVPDPQPLGQ |
| hu.27 | PPAAPSGLGTNTMASGSGAPMADNNEGADGVGNSSGNWHCDSTWMGDRVITTSTRTWALP |
| (AAS9927 | TYNNHLYKQISSQSGASNDNHYFGYSTPWGYFDFNRFHCHFSPRDWQRLINNNWGFRPKRLSF |
| 7)) | KLFNIQVKEVTQNDGTTTIANNLTSTVQVFTDSGYQLPYVLGSAHQGCLPPFPADVFMVPQYGY |
| | LTLNNGSQAVGRSSFYCLEYFPSQMLRTGNNFTFSYTFEDVPFHSSYAHGQSLDRLMNPLIDQYL |
| | YYLSRTNTPSGTTTMSRLQFSQAGASDVRDQSRNWLPGPCYRQQRVSKTAADNNNSDYSWTG |
| | ATKYHLNGRDSLVNPGPAMASHKDDEEKYFPQSGVLVFGKQDSGKTNVDIEKVMITDEEEIRTT |
| | NPAATEQYGSVSTNLQSGNTQAATSDVNTQGVLPGMVWQDRDVYLQGPIWAKIPHTDGHFH |
| | PSPLMGGFGLKHPPPQILIKNTPVPANPSTTFSAAKFVSFITQYSTGQVSVEIEWELQKENSKRWN |
| | PEIQYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 84 | PVNEADAAALEHDKAYDRQLDSGDNPYLKYNHADAEFQERLKEDTSFGGNLGRAVFQAKKRVL |
| (AAV | EPLSLVEEPVKTAPGKKRPVEHSPAEPDSSSGTGKSGNQPARKRLNFGQTGDSDSVPDPQPLGQ |
| hu.28 | PPAAPSGLGTNTMATGSGAPMADNNEGADGVGNSSGNWHCDSTWMGDRVITTSTRTWALP |
| (AAS9927 | TYNNHLYKQISSQSGASNDNHYFGYSTPWGYFDFNRFHCHFSPRDWQRLINNNWGFRPKRLNF |
| 8)) | KLFNIQVKEVTQNDGTTTIANNLTSTVQVFTDSEYQLPYVLGSAHQGCLPPFPADVFMVPQYGYL |
| | TLNNGSQAVGRSSFYCLEYFPSQMLRTGNNFTFSYTFEDVPFHSSYAHSQSLDRLMNPLIDQYLY |
| | YLSRTNTPSGTTTQSRLQFSQAGASDIQDQSRNWLPGPCYRQQRVSKTSADNNNSEYSWTGAT |
| | KYHLNGRDSLVNPGPAMASHKDDEEKFFPQSGVLIFGKQGSEKTNVDIEKVMITDEEEIRTTNPV |
| | ATEQYGSVSTNLQSGNTQAATADVNTQGVLPGMVGQDRDVYLQGPTWAKIPHTDGHFHPSPL |
| | MGGFGLKHPPPQILIKNTPVPANPSTTFSAAKFASFITQYSTGQVSVEIEWELQKENSKRWNPEIQ |
| | YTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 85 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPAEPDSSSGTGKSGNQPARKRLNFGQTGDSDSVPDPQPLGQ |
| hu.29 | PPAAPSGLGTNTMATGSGAPMADNNEGADGVGNSSGNWHCDSTWMGDRVITTSTRTWALP |
| (AAS9927 | TYNNHLYKQISSQSGASNDNHYFGYSTPWGYFDFNRFHCHFSPRDWQRLINNNWGFRPKRLNF |
| 9)) | KLFNIQVKEVTQNDGTTTIANNLTSTVQVFTDSEYQLPYVLGSAHQGCLPPFPADVFMVPQYGYL |
| | TLNNGSQAVGRSSFYCLGYFPSQMLRTGNNFTFSYTFEDVPFHSSYAHSQSLDRLMNPLIDQYLY |
| | YLSRTNTPSGTTTQSRLQFSQAGASDIRDQSRNWLPGPCYRQQRVSKTSADNNNSEYSWTGAT |
| | KYHLNGRDSLVNPGPAMASHKDDEEKFFPQSGVLIFGKQGPEKTNVDIEKVMITDEEEIRTTNPV |
| | ATEQYGSVSTNLQSGNTQAATADVNTQGVLPGMVWQDRDVYLQGPIWAKIPHTDGHFHPSPL |
| | MGGFGLKHPPPQILIKNTPVPANPSTTFSAAKFASFITQYSTGQVSVEIEWELQKENSKRWNPEIQ |
| | YTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPGNGLDKGE |
| NO: 86 | PVNAADAAALEHDKAYDQQLKAGDNPYLKYNHADAEFQERLKEDTSFGGNLGRAVFQAKKRLL |
| (AAV | EPLGLVEEAAKTAPGKKRPVEQSPQEPDSSAGIGKSGSQPAKKKLNFGQTGDTESVPDPQPIGEP |
| hu.31 | PAAPSGVGSLTMASGGGAPVADNNEGADGVGSSSGNWHCDSQWLGDRVITTSTRTWALPTY |
| (AAS9928 | NNHLYKQISNSTSGGSSNDNAYFGYSTPWGYFDFNRFHCHFSPRDWQRLINNNWGFRPKRLNF |
| 1)) | KLFNIQVKEVTDNNGVKTIANNLTSTVQVFTDSDYQLPYVLGSAHEGCLPPFPADVFMIPQYGYL |
| | TLNDGGQAVGRSSFYCLEYFPSQMLRTGNNFQFSYEFENVPFHSSYAHSQSLDRLMNPLIDQYLY |
| | YLSKTINGSGQNQQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKVMITNEEEIKTTNP |
| | VATESYGQVATNHQSAQAQAQTGWVQNQGILPGMVWQDRDVYLQGPIWAKIPHTDGNFHP |
| | SPLMGGFGMKHPPPQILIKNTPVPADPPTAFNKDKLNSFITQYSTGQVSVEIEWELQKENSKRW |
| | NPEIQYTSNYYKSNNVEFAVSTEGVYSEPRPIGTRYLTRNL |
| SEQ ID | MAADGYLPDWLEDTLSEGIRQWWKLKPGPPPPKPAERHKDDSRGLVLPGYKYLGPGNGLDKGE |
| NO: 87 | PVNAADAAALEHDKAYDQQLKAGDNPYLKYNHADAEFQERLKEDTSFGGNLGRAVFQAKKRLL |
| (AAV | EPLGLVEEAAKTAPGKKRPVEQSPQEPDSSAGIGKSGSQPAKKKLNFGQTGDTESVPDPQPIGEP |
| hu.32 | PAAPSGVGSLTMASGGGAPVADNNEGADGVGSSSGNWHCDSQWLGDRVITTSTRTWALPTY |
| (AAS9928 | NNHLYKQISNSTSGGSSNDNAYFGYSTPWGYFDFNRFHCHFSPRDWQRLINNNWGFRPKRLNF |
| 2)) | KLFNIQVKEVTDNNGVKTIANNLTSTVQVFTDSDYQLPYVLGSAHEGCLPPFPADVFMIPQYGYL |
| | TLNDGSQAVGRSSFYCLEYFPSQMLRTGNNFQFSYEFENVPFHSSYAHSQSLDRLMNPLIDQYLY |
| | YLSKTINGSGQNQQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKVMITNEEEIKTTNP |
| | VATESYGQVATNHQSAQAQAQTGWVQNQGILPGMVWQDRDVYLQGPIWAKIPHTDGNFHP |
| | SPLMGGFGMKHPPPQILIKNTPVPADPPTAFNKDKLNSFITQYSTGQVSVEIEWELQKENSKRW |
| | NPEIQYTSNYYKSNNVEFAVNTEGVYSEPRPIGTRYLTRNL |
| SEQ ID | MAADGYLPDWLEDTLSEGIRQRWKLKPGPPPPEPAERHKDDSRGLVLPGYKYLGPFNGLDKGEP |
| NO: 88 | VNEADAAALEHDKAYDRQLDSGDNPYLKYNHADAEFQERLKEDTSFGGNLGRAVFQAKKRVLE |
| (AAV | PLGLVEEPVKTAPGKKRPVEHSPVEPDSSSGTGKAGQQPARKRLNFGQTGDADSVPDPQPLGQ |
| hu.34 | PPAAPSGLGTNTMATGSGAPMADNNEGADGVGNSSGNWHCDSTWMGDRVITTSTRTWALP |
| (AAS9928 | TYNNHLYKQISSQSGASNDNHYFGYSTPWGYFDFNRFHCHFSPRDWQRLINNNWGFRPKRLNF |
| 3)) | KLFNIQVKEVTQNDGTTTIANNLTSTVQVFTDSEYQLPYVLGSAHQGCLPPFPADVFMVPQYGYL |
| | TLNNESQAVGRSSFYCLEYFPSQMLRTGNNFTFSYTFEDVPFHSSYAHSQSLGRLMNPLIDQYLYY |
| | LSRTNTPSGTTTQSRLQFSQAGASDIRDQSRNWLPGPCYRQQRVSKTSADNNNSEYSWTGATK |
| | YHLNGRDSLVNPGPAMASHKDDEEKFFPQSGVLIFGKQGSEKTNVDIEKVMITDEEEIRTTNPVA |
| | TEQYGSVSTNLQRGNRQAATADVNTQGVLPGMVWQDRDVYLQGPIWAKIPHTDGHFHPSPL |
| | MGGFGLKHPPPQILIKNTPVPANPSTTFSAAKFASFITQYSTGQVSVEIEWELQKENSKRWNPEIQ |
| | YTSNYNKSVNVDFTVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 89 | GEPVNAADAAALEHDKAYDQQLKAGDNPYLRYNHADAEFQERLQEDTSFGGNLGRAVFQAKK |
| (AAVhu.3 | RVLEPLGLVEEAAKTAPGKKRPVEPSPQRSPDSSTGIGKKGQQPAKKRLNFGQTGDSESVPDPQP |
| 7 | IGEPPAGPSGLGSGTMAAGGGAPMADNNEGADGVGSSSGNWHCDSTWLGDRVITTSTRTWA |
| (AAS9928 | LPTYNNHLYKQISNGTSGGSTNDNTYFGYSTPWGYFDFNRFHCHFSPRDWQRLINNNWGFRPK |
| 5)) | RLSFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFEFSYTFEDVPFHSSYAHSQSLDRLMNPLIDQ |
| | YLYYLSRTQSTGGTQGTQQLLFSQAGPANMSAQAKNWLPGPCYRQQRVSTTLSQNNNSNFAW |
| | TGATKYHLNGRDSLVNPGVAMATHKDDEERFFPSSGVLMFGKQGAGRDNVDYSSVMLTSEEEI |
| | KTTNPVATEQYGVVADNLQQTNTGPIVGNVNSQGALPGMVWQNRDVYLQGPIWAKIPHTDG |
| | NFHPSPLMGGFGLKHPPPQILIKNTPVPADPPTTFSQAKLASFITQYSTGQVSVEIEWELQKENSK |
| | RWNPEIQYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 90 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPVEPSPQRSPDSSTGIGKKGQQPARKRLNFGRTGDSESVPDPQP |
| hu.39 | IGEPPAAPSSVGSGTMAAGGGAPMADNNEGADGVGSSSGNWHCDSTWLGDRVITTSTRTWA |
| (AAS9928 | LPTYNNHLYKQISNGTSGGSTNDNTYFGYSTPWGYLDFNRFHCHFSPRDWQRLINNNWGFRPK |
| 6)) | RLSFKLFNIQVKEVTQNEGTKTIANNLASTIQVFTDSEYQPPYVLGSAHQGCLPPFPADVFMIPQY |
| | GYLTLNNGSQAVGRSSFYCLEYFPSQMLRTGNNFSFSYTFEDVPFHSSYAHSQSLDRLMNPLIDQ |
| | YLYYLSRTQSTGGTAGTQQLLFSRAGPSNMSAQARNWLPGPCYRQQRVSTTLSQNNNSNFAW |
| | TGATKYHLNGRDSLVNPGVAMATNKDDEDRFFPSSGILMFGKQGAGKDNVDYSNVMLTSEEEI |
| | KTTNPVATEQYGVVADNLQQQNTAPTVGAVNSQGALPGMVWQNRDVYLQGPIWAKIPHTD |
| | GNFHPSPLMGGFGLKHPPPQILIKNTPVPADPPTAFNQAKLNSFIAQYSTGQVSVEIEWELQKEN |
| | SKRWNPEIQYTSNYYKSTNADFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 91 | GEPVNAADAAALEHDKAYDQQLKAGDNPYLRYNHADAEFQERLQEDTSFGGNLGRAVFQAKK |
| (AAV | RVLEPLGPVEEAAKTAPGKKRPVEPPPQRSPDSSTGIGKKGQQPAKKRLNFGQTGDSESVPDPQ |
| hu.41 | PIGEPPAGPSGLGSGTMAAGGGAPMADNNEGADGVGSSSGNWHCDSTWLGDRVITTSTRTW |
| (AAS9928 | ALPTYNNHLYKQISNGTSGGSTNDNTYFGYSTPWGYFDFNRFHCHFSPRDWQRLINNNWGFRP |
| 9)) | KRLSFKLFNIQVKEVTQNEGTKTVANNLTSTIQVFTDSEYQLPYVLGSAHQGCLPPFPADVFMIPQ |
| | YGYLTLNNGSQAVGRSSFYCLEYFPSQMLRTGNNFEFSYTFEDVPFHSSYAHSQSLDRLMNPLID |
| | QYLYYLSRTQSTGGTQGTQQLLFSQAGPANMSAQAKNWLPGPCYRQQRVSTTLSQNNNSNFA |
| | WTGATKYHLNGRDSLVNPGVAMATHKDDEERFFPSSGVLMFGKQGAGRDNVDYSSVMLTSEE |
| | EIKTTNPVATEQYGVVADNLQQTNTGPIVGNVNSQGALPGMVWQNRDVYLQGPIWAKIPHTD |
| | GNFHPSPLMGGFGLKHPPPQILIKNTPVPADPPTTFSQAKLASFITQYSTGQVSVEIEWELQKENS |
| | KRWNPEIQYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 92 | GEPVNAADAAALEHDKAYDQQLKAGDNPYLRYNHADAEFQERLQEDTSFGGNLGRAVFQAKK |
| (AAV | RVLEPLGLVEEAAKTAPGKKRPVEPSPQRSPDSSTGIGKKGQQPAKKRLNFGQTGDSESVPDPQP |
| hu.42 | IGEPPAGPSGLGSGTMAAGGGAPMADNNEGADGVGSSSGNWHCDSTWLGDRVITTSTRTWA |
| (AAS9929 | LPTYNNHLYKQISNGTSGGSTNDNTYFGYSTPWGYFDFNRFHCHFSPRDWQRLINNNWGFRPK |
| 0)) | RLSFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFEFSYTFEDVPFHSSYAHSQSLDRLMNPLIDQ |
| | YLYYLSRTQSTGGTQGTQQLLFSQAGPANMSAQAKNWLPGPCYRQQRVSTTLSQSNNSNFAW |
| | TGATKYHLNGRDSLVNPGVAMATHKDDEERFFPSSGVLMFGKQGAGRDNVDYSSVMLTSEEEI |
| | KTTNPVATEQYGVVADNLQQTNTGPIVGNVNSQGALPGMVWQNRDVYLQGPIWAKIPHTDG |
| | NFHPSPLMGGLGLKHPPPQILIKNTPVPADPPTTFSQAKLASFITQYSTGQVSVEIEWELQKENSK |
| | RWNPEIQYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 93 | GEPVNAADAAALEHDKAYDQQLKAGDNPYPRYNHADAEFQERLQEDTPFGGNLGRAVFQAKK |
| (AAV | RVLEPLGLVEEAAKTAPGKKRPVEPSPQRSPDSSTGIGKKGQQPAKKRLNFGQTGDSESVPDPQP |
| hu.43 | IGEPPAGPSGLGSGTMAAGGGAPMADNNEGADGVGNASGNWHCDSTWLGDRVITTSTRTW |
| (AAS9929 | ALPTYNNHLYKQISSASTGASNDNHYFGYSTPWGYFDFNRFHCHFSPRDWQRLINNNWGFRPK |
| 1)) | RLNFKLFNIQVKEVTTNDGVTTIANNLTSTVQVFSDSEYQLPYVLGSAHQGCLPPFPADVFMIPQY |
| | GYLTLNNGSQAVGRSSFYCLEYFPSQMLRTGNNFTFSYTFEEVPLHSSYAHSQSLDRLMNPLIVQY |
| | LYYLNRTQNQSGSAQNKDLLFSRGSPAGMSVQPKNWLPGPCYRQQRVSKTKTDNNNSNFTWT |
| | GASKYNLNGRESIINPGTAMASHKDDEDKFFPMSGVMIFGKESAGASNTALDNVMITDEEEIKA |
| | TNPVATERFGTVAVNFQSSSTDPATGDVHAMGALPGMVWQDRDVYLQGPIWAKIPHTDGHF |
| | HPSPLMGGFGLKNPPPQILIKNTPVPANPPAEFSATKFASFITQYSTGQVSVEIEWELQKENSKR |
| | WNPEVQYTSNYAKSASVDFTVDNNGLYTEPRPIGTRYLTRPL |
| SEQ ID | MAADGYLPDWLEDTLSEGIRQWWKLRPGPPPPKPAERHKDDSRGLVLPGYKYLGPFNGLDKGE |
| NO: 94 | PVNEADAAALEHDKAYDRQLDSGDNPYLKYNHADAEFQERLKEDTSFGGNLGRAVFQAKKRVL |
| (AAV | EPLGLVEEGAETAPGKKRPVEQSPQGPDSSSGIGKTGQQPAKKRLNFGQTGDSESVPDPQPLGE |
| hu.44 | PPATPAAVGPTTMASGGGAPMADNNEGADGVGNASGNWHCDSTWLGDRVITTSTRTWALP |
| (AAS9929 | TYNNHLYKQISSASTGASNDNHYFGYSTPWGYFDFNRFHCHFSPRDWQRLINNNWGFRPKRLN |
| 2)) | FKLFNIQVKEVTTNDGVTTIANNLTSTVQVFSDSEYQLPYVLGSAHQGCLPPFPADVFMIPQYGYL |
| | TLNNGSQAVGRSSFYCLEYFPSQMLRTGNNFTFSYTFEEVPFHSSYAHSQSLDRLMNPLIDQYLYY |
| | PNRTQNQSGSAQNKDLLFSRGSPAGMSVQPKNWLPGPCYRQQRVSKTKTDNNNSNFTWTGA |
| | SKYNLNGRESIINPGTAMASHKDDEDKFFPMSGVMIFGKESAGASNTALDNVMITDEEEIKATN |
| | PVATERFGTVAVNFQSSSTDPATGDVHAMGALPGMVWQGRDVYLQGPIWAKIPHTDGHFHP |
| | SPLMGGFGLKNPPPQILIKNTPVPANPPAEFSATKFASFITQYSTGQVSVEIEWELQKENSKRWN |
| | PEVQYTSNYAKSANVDFTVDNNGLYTEPRPIGTRYLTRPL |
| SEQ ID | MAADGYLPDWLEDTLSEGIRQWWKLKPGPPPPKPAERHRDDSRGLVLPGYKYLGPFNGLDKGE |
| NO: 95 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPVEPDSSSGTGKAGQQPARKRLNFGQTGDADSVPDPQPLG |
| hu.45 | QPPAAPSGLGTNTMATGSGAPMADNNEGADGVGNSSGNWHCDSTWMGDRVITTSTRTWAL |
| (AAS9929 | PTYNNHLYKQISSQSGASNDNHYFGYSTPWGYFDFNRFHCHFSPRDWQRLINNNWGFRPKRLN |
| 3)) | FKLFNIQVKEVTQNDGTTTIANNLTSTVQVFTDSGYQLPYVLGSAHQGCLPPFPADVFMVPQYG |
| | YPTLNNGSQAVGRSSFYCLEYFPSQMLRTGNNFTFSYTFEDVPFHSSYAHSQSLDRLMNPLIDQY |
| | LYYLSTTNTPSGTTTQSRLQFSQAGASDIRDQSRNWLPGPCYRQQRVSKTSADNNNSEYSWTGA |
| | TKYHLNGRDSLVNPGPAVASHKDDEEKFFPQSGVLIFGKQGSEKTNVDIEKVMITDEEEIRTTNPV |
| | ATEQYGSVSTNLQRGNRQAATADVNTQGVLPGMVWQDRDVYLQGPIWAKIPHTDGHFHPSP |
| | LMGGFGLKHPPPQILIKNTPVPANPSTTFSAAKFASFITQYSTGQVSVEIEWELQKENSKRWNPEI |
| | QYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 96 | PVNEADAAALEHDKAYDRQLDSGDNPYLKYNHADAEFQERLKEDTSFGGNLGRAVFQAKKRVL |
| (AAV | EPLGLVEEGAKTAPGKKRPVEQSPQEPDSPSGIGKTGQQPAKKRLNFGQTGDSESVPDPQPLGE |
| hu.46 | PPATPAAVGPTTMASGGGAPMADNNEGADGVGNASGNWHCDSTWLGDRVITTSTRTWALP |
| (AAS9929 | TYNNHLYKQISSASTGASNDNHYFGYSTPWGYFDFNRFHCHFSPRDWQRLINNNWGFRPKRLN |
| 4)) | FKLFNIQVKEVTTNDGVTTIANNLTSTVQVFSDSEYQLPYVLGSAHQGRLPPFPADVFMIPQYGYL |
| | TLNNGSQAVGRSSSYCLEYFPSQMLRTGNNFTFSYTFEEVPLHSSCAHSQSLDRLMNPLIDQYLYY |
| | LNRTQNQSGSAQNRDLLFSRGSPAGMSVQPKNWLPGPCYRQQRVSKTKTDNNNSNFTWTGA |
| | SKYNLNGRESIINPGTAMASHKDDEDKFFPMSGVMIFGKESAGASNTALDNVMITDEEEIKATN |
| | PVATERFGTVAVNFQSSSTDPATGDVHAMGALPGMVWQDRDVYLQGPIWAKIPHTDGHFHP |
| | SPLMGGFGLKNPPPQILIKNTPVPANPPAEFSATKFASFITQYSAGQVSVEIEWELQKENSKRWN |
| | PEVQYTSNYAKSANVDFTVDNNGLYTEPRPIGTRYLTRPL |
| SEQ ID | MAADGYLPDWLEDTLSEGIRQWWKLKPGPPPPKPAERHRDDSRGLVLPGYKYLGPFNGLDKGE |
| NO: 97 | PVNEADAAALEHDKAYDRQLDSGDNPYLKYNHADAEFQERLKEDTSFGGNLGRAVFQAKKRVL |
| (AAV | EPLGLVGEPVKTAPGKKRPVEHSPVEPDSSSGTGKAGQQPARKRLNFGQTGDADSVPDPQPLG |
| hu.47 | QPPAAPSGLGTNTMATGSGAPMADNNEGADGVGNSSGNWHCDSTWMGDRVITTSTRTWAL |
| (AAS9929 | PTYNNHLYKQISSQSGASNDSHYFGYSTPWGYFDFNRFHCHFSPRDWQRLINNNWGFRPKRLN |
| 5)) | FKLFNIQVKEVTQNDGTTTIANNLTSTVQVFTDSEYQLPYVLGSAHQGCLPPFPADVFMVPQYGY |
| | LTLNNGSQAVGRSSFYCLEYFPSQMLRTGNNFTFSYTFEDVPFHSSYAHSQSLDRLMNPLIDQYLY |
| | YLSTTNTPSGTTTQSRLQFSQAGASDIRDQSRNWLPGPCYRQQRVSKTSADNNNSEYSWTGAT |
| | KYHLNGRDSLVNPGPAMASHKDNEEKFFPQSGVLIFGKQGSEKTNVDIEKVMITDEEEIRTTNPV |
| | ATEQYGSVSTNLQRGNRQAATADVNTQGVLPGMVWQDRDVYLQGPIWAKIPHTDGHFHPSP |
| | LMGGFGLKHPPPQILIKNTPVPANPSTTFSAAKFASFITQYSTGQVSVEIEWELQKENSKRWNPEI |
| | QYTSNYNKSVNVDFTVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 98 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPVEQSPQEPDSSSGIGKTGQQPAKKRLNFGQTGDSESVPDPQPL |
| hu.48 | GEPPATPAAVGPTTMASGGGAPMADNNEGADGVGNASGNWHCDSTWLGDRVITTSTRTWA |
| (AAS9929 | LPTYNNHLYKQISSTSTGASNDNHYFGYGTPWGYFDFNRFHCHFSPRDWQRLINNNWGFRPKR |
| 6)) | LNFKLFNIQVEEVTTNDGVTTIANNLTSTVQVFSDSEYQLPYVLGSAHQGCLPPFPADVFMIPQY |
| | GYLTLNNGSQAVGRSSFYCLEYFPSQMLRTGNNFTFSYTFEEVPFHSSYAHSQSLDRLMNPLIDQ |
| | YLYYLNRTQNQSGSAQNKDLLFSRGSPAGMSVQPKNWLPGPCYRQQRVSKTKTDNNNSNFTW |
| | TGASKYNLNGRESIINPGTAVASHKDDEDKFFPMSGVMIFGKESAGASSTALDNVMITDEEEIKA |
| | TNPVATERFGTVAVNFQSSSTDPATGDVHAMGALPGMVWQDRDVYLQGPIWAKIPHTDGHF |
| | HPSPLMGGFGLKNPPPQILIKNTPVPANPPAEFSATKFASFITQYSTGQVSVEIEWELQKENSKR |
| | WNPEVQYTSNYAKSANVDFTVDNNGLYTEPRPIGTRYLTRPL |
| SEQ ID | MAADGYLPDWLEDTLSEGIRQWWKLKPGPPPPKPAERHKDDSRGLVLPGYKYLGPFNGLDKGE |
| NO: 99 | PVNEADAAALEHDKAYDRQLDSGDNPYLKYNHADAEFQERLKEDTSFGGNLGRAVFQAKKRVL |
| (AAV | EPLGLVGEPVKTAPGKKRPVEHSPVEPDSSSGTGKAGQQPARKRLNFGQTGDADSVPDPQPLG |
| hu.51 | QPPAAPSGLGTNTMATGSGAPMADNNEGADGVGNSSGNWHCDSTWMGDRVITTSTRTWAL |
| (AAS9929 | PTYNNHLYKQISSQSGASNDNHYFGYSTPWGYFDFNRFHCHFSPRDWQRLINNNWGFRPKRLN |
| 8)) | FKLFNIQVKEVTQNDGTTTIANNLTSTVQVFTDSEYQLPYVLGSAHQGCLPPFPADVFMVPQYGY |
| | LTLNNGSQAVGRSSFYCLEYFPSQMLRTGNNFTFSYTFEDVPFHSGYAHSQSLDRLMNPLIDQYL |
| | YYLSTTNTPSGTTTQSRLQFSQAGASDIRDQSRNWLPGPCYRQQRVSKTSADNNNSEYSWTGAT |
| | KYHLNGRDSLVNPGPAMASHKDNEEKFFPQSGVLIFGKQGSEKTNVDIEKVMITDEEEIRTTNPV |
| | ATEQYGSVSTNLQRGNRQAATADVNTQGVLPGMVWQDRDVYLQGPIWAKIPHTDGHFHPSP |
| | LMGGFGLKHPPPQILIKNTPVPANPSTTFSAAKFASFITQYSTGQVSVEIEWELQKENSKRWNPEI |
| | QYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 100 | PVNEADAAALEHDKAYDRQLDSGDNPYLKYNHADAEFQERLKEDTSFGGNLGRAVFQAKKRVL |
| (AAV | EPLGLVGEPVKTAPGKKRPVEHSPVEPDSSSGTGKAGQQPARKRLNFGQTGDADSVPDPQPLG |
| hu.52 | QPPAAPSGLGTNTMATGSGAPMADNNEGADGVGNSSGNRHCDSTWMGDRVITTSTRTWAL |
| (AAS9929 | PTYNNHLYRQISSQSGASNDNHYFGYSTPWGYFDFNRFHCHFSPRDWQRLINNNWGFRPKRLN |
| 9)) | FKLFNIQVKEVTQNDGTTTIANNLTSTVQVFTDSEYQLPYVLGSAHQGCLPPFPADVFMVPQYGY |
| | LTLSNGSQAVGRSSFYCPEYFPSQMLRTGNNFTFSYTFEDVPFHSSYAHSQSLDRLMNPLIDQYLY |
| | YLSTTNTPSGTTTQSRLQFSQAGASDIRDQSRNWLPGPCYRQQRVSKTSADNNNSEYSWTGAT |
| | KYHLNGRDSLVNPGPAMASHKDNEEKFFPQSGVLIFGKQGSEKTNVDIEKVMITDEEEIRTTNPV |
| | ATEQYGSVSTNLQRGNRQAATADVNTQGVLPGMVWQDRDVYLQGPIWAKIPHTDGHFHPSP |
| | LMGGFGPKHPPPQILIKNTPVPANPSTTFSAAKFASFITQYSTGQVSVEIEWELQKENSKRWNPEI |
| | QYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 101 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPAEPDSSSGTGKAGQQPARKRLNFGQTGDADSVPDPQPLRQ |
| hu.53 | PPAAPTSLGSTTMATGSGAPMADNNEGADGVGNSSGNWHCDSQWLGDRVITTSTRTWALPT |
| (AAS9930 | YNNHLYKQISSQSGASNDNHYFGYSTPWGYFDFNRFHCHFSPRDWQRLINNNWGFRPKRLNFK |
| 0)) | LFNIQVKEVTQNDGTTTIANNLTSTVQVFTDSEYQLPYVLGSAHQGCLPPFPADVFMVPQYGYLT |
| | LNNGSQAVGRSSFYCLEYFPSQMLRTGNNFQFSYTFEDVPFHSSYAHSQSLDRLMNPLIDQYLYY |
| | LNRTQTASGTQQSRLLFSQAGPTSMSLQAKNWLPGPCYRQQRLSKQANDNNNSNFPWTGATK |
| | YYLNGRDSLVNPGPAMASHKDDEEKFFPMHGTLIFGKEGTNATNAELENVMITDEEEIRTTNPV |
| | ATEQYGYVSNNLQNSNTAASTETVNHQGALPGMVWQDRDVYLQGPIWAKIPHTDGHFHPSPL |
| | MGGFGLKHPPPQIMIKNTPVPANPPTNFSSAKFASFITQYSTGQVSVEIEWELQKENSKRWNPEI |
| | QYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 102 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPAEPDSSSGTGKAGQQPARKRLNFGQTGDADSVPDPQPLG |
| hu.54 | QPPAAPTSLGSTTMATGSGAPMADNNEGADGVGNSSGNWHCDSQWLGDRVITTSTRTWALP |
| (AAS9930 | TYNNHLYKQISSQSGASNDNHYFGYSTPWGYFDFNRFHCRFSPRDWQRLINNNWGFRPKRLNF |
| 1)) | KLFNIQVKEVTQNDGTTTIANNLTSTVQVFTDSEYQLPYVLGSAHQGCLPPFPADVFMVPQYGYL |
| | TLNNGSQAVGRSSFYCLEYFPSQMLRTGNNFQFSYTFEDVPFHSSYAHSQGLDRLMNPLIDQYLY |
| | YLNRTQTASGTQQSRLLFSQAGPTSMSLQAKNWLPGPCYRQQRLSKQANDNNNSNFPWTGAT |
| | KYHLNGGDSLVNPGPAMASHKDDEEKFFPMHGTLIFGKEGTNATNAELENVMITDEEEIRTTNP |
| | VATEQYGYVSNNLQNSNTAASTETVNHQGALPGMVWQDRDVYLRGPIWAKIPHADGHFHPSP |
| | LMGGFGLKHPPPQIMIKNTPVPANPPTNFSSAKFASFITQYSTGQVSVEIEWELQKENSKRWNPE |
| | IQYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 103 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPAEPDSSSGTGKAGQQPARKRLNFGQTGDADSVPDPQPLG |
| hu.55 | QPPAAPTSLGSTTMATGSGAPMADNNEGADGVGNSSGNWHCDSQWLGDRVITTSTRTWALP |
| (AAS9930 | TYNNHLYKQISSQSGASNDNHYFGYSTPWGYFDFNRFHCHFSPRDWQRLINNNWGFRPKRLNF |
| 2)) | KLFNIQVKEVTQNDGTTTIANNLTSTVQVFTDSEYQLPYVLGSAHQGCLPPFPADVFMVPQYGYL |
| | TLNNGSQAVGRSSFYCLECFPSQMLRTGNNFQFSYTFEDVPFHSSYAHSQSLDRLMNPLIDQYLY |
| | YLNRTQTASGTQQSRLLFSQAGPTSMSLQAKNWLPGPCYRQQRLSKQANDNNNSNFPWTGAT |
| | KYHLNGRDSLVNPGPAMASHKDDEEKFFPMHGTLIFGKEGTNATNAELENVMITDEEEIRTTNP |
| | VATEQYGYVSNNLQNSNTAASTETVNHQGALPGMVWQDRDVYLQGPIWAKIPHTDGHFHPS |
| | PLMGGFGLKHPPPQIMIKNTPVPANPPTNFSSAKFASFITQYSTGQVSVEIEWELQKENSKRWN |
| | PEIQYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 104 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPVEPDSSSGTGKAGNQPARKRLNFGQTGDADSVPDPQPLGQ |
| hu.56 | PPASPSGLGTNTMATGSGAPMADNNEGADGVGNSSGNWHCDSTWMGDRVVTTSTRTWAL |
| (AAS9930 | PTYNNHLYKQISSQSGASNDNHYFGYSTPWGYFDFNRFHCHFSPRDWQRLINNNWGFRPKRLN |
| 3)) | FKLFNIQVKEVTQNDGTTTIANNLTSTVQVFTDLEYQLPYVLGSAHQGCLPPFPADVFMVPQYGY |
| | LTLNNGSQAVGRSSFYCLEYFPSQMLRTGNNFTFSYTFEDVPFHSSYAHSQSLDRLMNPLIDQYLY |
| | YLSRTNTPSGTTTQSRLQFSQAGASDIRDQSRNWLPGPCYRQQRVSKTAADNNNSEYSWTGAT |
| | KYHLNGRDSLVNPGPAMASHKDDEEKFFPQSGVLIFGKQGSEKTNVDIEKVMITDEEEIRTTNPV |
| | ATEQYGSVSTNLQSGNTQAATSDVNTQGVLPGMVWQDRDVYLQGPIWAKIPHTDGHFHPSPL |
| | MGGFGLKHPPPQILIKNTPVPANPSTTFSAAKFASFITQYSTGQVSVEIEWELQKENSKRWNPEIQ |
| | YTSNYNKSVNVDFTVDTNGVYSEPRPIGTRYLTRNL |
| SEQ ID | MAADGYLPDWLEDTLSEGIRQWWKLKPGPPPKPAERHKDDSRGLVLPGYKYLGPFNGLDKGEP |
| NO: 105 | VNEADAAALEHDKAYDRQLDSGDNPYLKYNHADAEFQERLKEDTSFGGNLGRAVFQAKKRVLE |
| (AAV | PLGLVEEPVKTAPGKKRPVEHSPVEPDSSSGTGKAGNQPARKRLNFGQTGDADSVPDPQPLGQ |
| hu.57 | PPAAPSGLGTNTMATGSGAPMADNNEGADGVGNSSGDWHCDSTWMGDRVITTSTRTWALP |
| (AAS9930 | TYNNHLYKQISSQSGASNDNHYFGYSTPWGYFDFNRFHCHFSPRDWQRLINNNWGFRPKRLNL |
| 4)) | KLFNIQVKEVTQNDGTTTIANNLTSTVQVFTDLEYQLPYVLGSAHQGCLPPFPADVFMVPQYGYL |
| | TLNNGSQAVGRSSFYCLEYFPSQMLRTGNNFTFSYTFEDVPFHSSYAHSQSLDRLMNPLIDQYLY |
| | YLSRTNTPSGTTTQSRLQFSQAGASDIRDQSRNWLPGPCYRQQRVSKTAADNNNGEYSWTGAT |
| | KYHLNGRDSLVNPGPAMASHKDDEEKFFPQSGVLIFGKQGSEKTNVDIEKVMITDEEEIRTTNPV |
| | ATEQYGSVSTNLQSGNTRAATSDVNTQGVLPGMVWQDRDVYLQGPIWAKIPHTDGHFHPSPL |
| | MGGFGLKHPPPQILIKNTPVPANPSTTFSAAKFASFITQYSTGQVSVEIEWELQKENSKRWNPEIQ |
| | YTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 106 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPVEPDSSSGTGKAGQQPARKRLNFGQTGDADSVPDPQPLG |
| hu.60 | QPPAAPSGLGSTTMATGSGAPMADNNEGADGVGNSSGNWHCDSQWLGDRVITTSTRTWAL |
| (AAS9930 | PTYNNHLYKQISSQSGASNDNHYFGYSTPWGYFDFNRFHCHFSPRDWQRLINNNWGFRPKRLN |
| 7)) | FKLFNIQVKEVTQNDGTTTIANNLTSTVQVFTDSEYQLPYVLGSAHQGCLPPFPADVFMVPQYGY |
| | LTLNNGSQAVGRSSFYCLEYFPSQMLRTGNNFQFSYTFEDVPFHSSYAHSQSLDRLMNPLVDQYL |
| | YYLNKTQTNSGTLQQSRLLFSQAGPTNMSLQAKNWLPGPCYRQQRLSKQANDNNNSNFPWTA |
| | ATKYHLNGRDSLVNPGPAMASHKDDEEKFFPMHGTLIFGKQGTNANDADLENVMITDEEEIRTT |
| | NPVATEQYGTVSNNLQNSNTGPTTGTVNHQGALPGMVWQDRDVYLQGPIWAKIPHTDGHFH |
| | PSPLMGGFGLKHPPPQIMIKNTPVPANPPTNFSSAKFASFITQYSTGQVSVEIEWELQKENSKRW |
| | NPEIQYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 107 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHPPVEPDSSSGTGKAGQQPARKRLNFGQTGDADSVPDPQPLG |
| hu.61 | QPPAAPSGLGSTTMATGSGAPMADNNEGADGVGNSSGNWHCDSQWLGDRVITTSTRTWAL |
| (AAS9930 | PTYNNHLYKQISSQSGASNDNHYFGYSTPWGYFDFNRFHCHFSPRDWQRLINNNWGFRPKRLN |
| 8)) | FKLFNIQVKEVTQNDGTTTIANNLTSTVQVFTDSEYQLPYVLGSAHQGCLPPFPADVFMVPQYGY |
| | LTLNNGSQAVGRSSFYCLEYFPSQMLRTGNNFQFSYTFEDVPFHSSYAHSQSLDRLMNPLIDQYL |
| | YYLNKTQTNSGTLQQSRLLFSQAGPTNMSLQAKNRLPGPCYRQQRLSKQANDNNNSNFPWTA |
| | ATKYHLNGRDSLVNPGPAMASHKDDEEKFFPMHGTLIFGKQGTNANDADLENVMITDEEEIRTT |
| | NPVATEQYGTVSNNLQNSNTGPTTGTVNHQGALPGMVWQDRDVYLQGPIWAKIPHTDGHFH |
| | PSPLVGGFGLKHPPPQIMIKNTPVPANPPTNFSSAKFASFITQYSTGQVSVEIEWELQKENSKRW |
| | NPEIQYTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 108 | PVNEADAAALEHDKAYDRQLDSGDNPYPKYNHADAEFQERLKEDTSFGGNLGRAVFQAKKRVL |
| (AAV | EPLGLVEEPVKTAPGKKRPVEHSPAEPDSSSGTGKAGQQPARKRLNFGQTGDADSVPDPQPLG |
| hu.63 | QPPAAPSGLGTNTMATGSGAPMADNNEGADGVGNSSGNWHCDSTWMGDRVITTSTRTWAL |
| (AAS9930 | PTYNNHLYKQISSQSGASNDNHYFGYSTPWGYFDFNRFHCHFSPRDWQRLINNNWGFRPKRLN |
| 9)) | FKLFNIQVKEVTQNDGTTTIANNLTSTVQVFTDSEYQLPYVLGSAHQGCLPPFPADVFMVPQYGY |
| | LTLNNGSQAVGRSSFYCLEYFPSQMLRTGNNFTFSYTFEDVPFHSSYAHSQSLDRLMNPLIDQYLY |
| | YLSRTNTPSGTTTQSRLQFSQAGASDIRDQSRNWLPGPCYRQQRVSKTSADNNNSEYSWTGAT |
| | KYHLNGRDSLVNPGPAMASHKDDEEKFFPQSGVLIFGKQDSGKTNVDIEKVMITDEEEIRTTNPV |
| | ATEQYGSVSTNLQSGNTQAATSDVNTQGVLPGMVWQDRDVYLQGPIWAKIPHTDGHFHPSPL |
| | MGGFGLKHPPPQILIKNTPVPANPSTTFSAAKFASFITQYSTGQVSVEIEWELQKENSKRWNPEIQ |
| | YTSNYNKSVNVDFTVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 109 | GEPVNAADAAALEHDKAYDQQLKAGDNPYLRYNHADAEFQERLQEDTSFGGNLGRAVFQAKK |
| (AAV | RVLEPLGLVEEAAKTAPGKKRPVEPSPQRSPDSSAGIGKKGQQPAKKRLNFGQTGDSESVPDPQP |
| hu.66 | IGEPPAGPSGLGSGTMAAGGGAPMADNNEGADGVGSSSGNWHCDSTWLGDRVITTSTRTWA |
| (AAS9931 | LPTYNNHLYKQISNGTSGGSTNDNTYFGYSTPWGYFDFNRFHCHFSPRDWQRLINNNWGFRPK |
| 1)) | RLSFKLFNIQVKEVTQNEGTETIANNLTSTIQVFTDSEYQLPYVLGSAHQGCLPPFPADVFMIPQY |
| | GYLTLNNGSQAVGRSSFYCLEYFPSQMLRTGNNFEFSYTFEDVPFHSSCAHSQSSDRLMNPLIDQ |
| | YLYYLSRTRSTGGTQGTQQLLFSQAGPANMSAQAKNWLPGPCYRQQRVSTTLSQNNNSNFAW |
| | TGATKYHLNGRDSLVNPGVAMATHKDDEERFFPSSGVLMFGKQGAGRDNVDYSSVMLTSEEEI |
| | KTTNPVATEQYGVVADNLQQTNTGPIVGNVNSQGALPGMVWQNRDVYLQGPIWAKIPHTDG |
| | NFHPSPLMGGFGLKHPPPQILIKNTPVPADPPTTFSQAKLASFITQYSTGQVSVEIEWELQKENSK |
| | RWNPEIQYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLLGYKYLGPFNGLDK |
| NO: 110 | GEPVNAADAAALEHDKAYDQQLKAGDNPYLRYNHADAEFQERLQEDTSFGGNLGRAVFQAKK |
| (AAV | RVLEPLGLVEEAAKTAPGKKRPVEPSPQRSPDSSTGIGKKGQQPAKKRLNFGQTGDSESVPDPQP |
| hu.67 | IGEPPAGPSGLGSGTMAAGGGAPMADNNEGADGVGSSSGNWHCDSTWLGDRVITTSTRTWA |
| (AAS9931 | LPTYNNHLYKQISNGTSGGSTNDNTYFGYSTPWGYFDFNRFHCHFSPRDWQRLINNNWGFRPK |
| 2)) | RLSFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFEFSYTFEDVPFHSGYAHSQSLDRLMNPLIDQ |
| | YLYYLSRTQSTGGTQGTQQLLFSQAGPANMSAQAKNWLPGPCYRQQRVSTTLSQNNNSNFAW |
| | TGATKYHLNGRDSLVNPGVAMATHKDDEERFFPSSGVLMFGKQGAGRDNVDYSSVMLTSEEEI |
| | KTTNPVATEQYGVVADNLQQTNTGPIVGNVNSQGALPGMVWQNRDVYLQGPIWAKIPHTDG |
| | NFHPSPLMGGFGLKHPPPQILIKNTPVPADPPTTFSQAKLASFITQYSTGQVSVEIEWELQKENSK |
| | RWNPEIQYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 111 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPVEPSPQRSPDSSTGIGKKGQQPAKKRLNFGQTGDSESVPDPQP |
| rh.10 | IGEPPAGPSGLGSGTMAAGGGAPMADNNEGADGVGSSSGNWHCDSTWLGDRVITTSTRTWA |
| (AAO882 | LPTYNNHLYKQISNGTSGGSTNDNTYFGYSTPWGYFDFNRFHCHFSPRDWQRLINNNWGFRPK |
| 01)) | RLNFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFEFSYQFEDVPFHSSYAHSQSLDRLMNPLIDQ |
| | YLYYLSRTQSTGGTAGTQQLLFSQAGPNNMSAQAKNWLPGPCYRQQRVSTTLSQNNNSNFAW |
| | TGATKYHLNGRDSLVNPGVAMATHKDDEERFFPSSGVLMFGKQGAGKDNVDYSSVMLTSEEEI |
| | KTTNPVATEQYGVVADNLQQQNAAPIVGAVNSQGALPGMVWQNRDVYLQGPIWAKIPHTDG |
| | NFHPSPLMGGFGLKHPPPQILIKNTPVPADPPTTFSQAKLASFITQYSTGQVSVEIEWELQKENSK |
| | RWNPEIQYTSNYYKSTNVDFAVNTDGTYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 112 | GEPVNEADAAALEHDKAYDKQLEQGDNPYLKYNHADAEFQERLQEDTSFGGNLGRAVFQAKKR |
| (AAV | VLEPLGLVEEGAKTAPGKKRPIESPDSSTGIGKKGQQPAKKKLNFGQTGDSESVPDPQPLGEPPA |
| rh.13 | APSGLGSGTMAAGGGAPMADNNEGADGVGNASGNWHCDSTWLGDRVITTSTRTWALPTYN |
| (AAO881 | NHLYKQISSQSGATNDNHFFGYSTPWGYFDFNRFHCHFSPRDWQRLINNNWGFRPRKLRFKLF |
| 99)) | NIQVKEVTTNDGVTTIANNLTSTIQVFSDSEYQLPYVLGSAHQGCLPPFPADVFMIPQYGYLTLNN |
| | GSQSVGRSSFYCLEYFPSQMLRTGNNFEFSYTFEEVPFHSSYAHSQSLDRLMNPLIDQYLYYLART |
| | QSTTGSTRELQFHQAGPNTMAEQSKNWLPGPCYRQQRLSKNIDSNNNSNFAWTGATKYHLNG |
| | RNSLTNPGVAMATNKDDEDQFFPINGVLVFGETGAANKTTLENVLMTSEEEIKTTNPVATEEYG |
| | VVSSNLQSSTAGPQTQTVNSQGALPGMVWQNRDVYLQGPIWAKIPHTDGNFHPSPLMGGFG |
| | LKHPPPQILIKNTPVPANPPEVFTPAKFASFITQYSTGQVSVEIEWELQKENSKRWNPEIQYTSNY |
| | AKSNNVEFAVNNEGVYTEPRPIGTRYLTRNL |
| SEQ ID | MAADGYLPDWLEDNLSEGIREWWDLKPGAPKPKANQQKQDDGRGLVLPGYKYLGPFNGLDK |
| NO: 113 | GEPVNEADAAALEHDKAYDKQLEQGDNPYLKYNHADAEFQERLQEDTSFGGNLGRAVFQAKKR |
| (AAV | VLEPLGLVEEGAKTAPGKKRPVEPSPQRSPDSSTGIGKTGQQPAKKRLNFGQTGDSESVPDPQPI |
| rh.19 | GEPPAGPSGLGSGTMAAGGGAPMADNNEGADGVGSSSGNWHCDSTWLGDRVITTSTRTWA |
| (AAO881 | LPTYNNHLYKQISNGTSGGSTNDNTYFGYSTPWGYFDFNRFHCHFSPRDWQRLINNNWGFRPR |
| 94)) | KLRFKLFNIQVKEVTTDDGVTTIANNLTSTIQVFSDSEYQLPYVLGSAHQGCLPPFPADVFMIPQY |
| | GYLTLNNGSQSVGRSSFYCLEYFPSQMLRTGNNFEFSYTFEEVPFHSSYAHSQSLDRLMNPLIDQY |
| | LYYLARTQSTTGSTRELQFHQAGPNTMAEQSKNWLPGPCYRQQRLSKNIDSNNNSNFAWTGA |
| | TKYHLNGRNSLTNPGVAMATNKDDEDQFFPINGVLVFGKTGAANKTTLENVLMTSEEEIKTTNP |
| | VATEEYGVVSSNLQSSTAGPQTQTVNSQGALPGMVWQNRDVYLQGPIWAKIPHTDGNFHPSP |
| | LMDGFGLKHPPPQILIKNTPVPANPPEVFTPAKFASFITQYSTGQVSVEIEWELQKENSKRWNPEI |
| | QYTSNYAKSNNVEFAVNNEGVYTEPRPIGTRYLTRNL |
| SEQ ID | MAADGYLPDWLEDNLSEGIREWWDLKPGAPKPKANQQKQDDGRGLVLPGYKYLGPFNGLDK |
| NO: 114 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPIESPDSSTGIGKKGQQPAKKKLNFGQTGDSESVPDPQPIGEPPA |
| rh.22 | GPSGLGSGTMAAGGGAPMADNNEGADGVGNASGNWHCDSTWLGDRVITTSTRTWALPTYN |
| (AAO881 | NHLYKQISSQSGATNDNHFFGYSTPWGYFDFNRFHCHFSPRDWQRLINNNWGFRPRKLRFKLF |
| 92)) | NIQVKEVTTNDGVTTIANNLTSTIQVFSDSEYQLPYVLGSAHQGCLPPFPADVFMIPQYGYLTLNN |
| | GSQSVGRSSFYCLEYFPSQMLRTGNNFEFSYTFEEVPFHSSYAHSQSLDRLMNPLIDQYLYYLART |
| | QSTTGSTRELQFHQAGPNTMAEQSKNWLPGPCYRRQRLSKDIDSNNNSNFAWTGATKYHLNG |
| | RNSLTNPGVAMATNKDDEDQFFPINGVLVFGKTGAANKTTLENVLMTSEEEIKTTNPVATEEYG |
| | VVSSNLQSSTAGPQTQTVNSQGALPGMVWQNRDVYLQGPIWAKIPHTDGNFHPSPLMGGFG |
| | LKHPPPQILIKNTPVPANPPEVFTPAKFASFITQYSTGQVSVEIEWELQKENSKRWNPEIQYTSNY |
| | AKSNNVEFAVNNEGVYTEPRPIGTRYLTRNL |
| SEQ ID | MAADGYLPDWLEDNLSEGIREWWDLKPGAPKPKANQQKQDDGRGLVLPGYKYLGPFNGLDK |
| NO: 115 | GEPVNEADAAALEHDKAYDKQLEQGDNPYLKYNHADAEFQERLQEDTSFGGNLGRAVFQAKKR |
| (AAV | VLEPLGLVEEGAKTAPGKKRPVEPSPQRSPDSSTGIGKTGQQPAKKRLNFGQTGDSESVPDPQPI |
| rh.23 | GEPPAGPSGLGSGTMAAGGGAPMADNNEGADGVGSSSGNWHCDSTWLGDRVITTSTRTWA |
| (AAO881 | LPTYNNHLYKQISNGTSGGSTNDNTYFGYSTPWGYFDFNRFHCHFSPRDWQRLINNNWGFRPK |
| 91)) | RLNFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFEFSYQFEDVPFHSSYAHSQSLDRLTNPLIDQY |
| | LYYLARTQSTTGSTRGLQFHQAGPNTMAEQSKNWLPGPCYRQQRLSKNIDSNNNSNFAWTGA |
| | TKYHLNGRNSLTNPGVAMATNKDDEDQFFPINGVLVFGKTGAANKTTLENVLMTSEEEIKTTNP |
| | VATEEYGVVSSNLQSSTAGPQTQTVNSQGALPGMVWQNRDVYLQGPIWAKIPHTDGNFHPSP |
| | LMGGFGLKHPPPQILIKYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 116 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPIESPDSSTGIGKKGQQPAKKKLNFGQTGDSESVPDPQPIGEPPA |
| rh.24 | GPSGLGSGTMAAGGGAPMADNNEGADGVGSSSGNWHCDSTWLGDRVITTSTRTWALPTYN |
| (AAO881 | NHLYKQISNGTSGGSTNDNTYFGYSTPWGYFDFNRFHCHFSPRDWQRLINNNWGFRPKRLNFK |
| 90)) | LFNIQVKEVTQNEGTKTIANNLTSTIQVFTDSEYQLPYVLGSAHQGCLPPFPADVFMIPQYGYLTL |
| | NNGSQAVGRSSFYCLEYFPSQMLRTGNNFEFSYQFEDVPFHSSYAHSQSLDRLMNPLIDQYLYYL |
| | SRTQSTGGTAGTQQLLFSQAGPNNMSAQAKNWLPGPCYRQQRVSTTVSQNNNSNFAWTGAT |
| | KYHLNGRDSLVNPGVAMATHKGDEERFFPSSGVLMFGKQGAGKDNVDYSSVMLTSEEEIKTTN |
| | PVATEQYGVVADNLQQQNAAPIVGAVNSQGALPGMVWQNRDVYLQGPIWAKIPHTDGNFHP |
| | SPLMGGFGLKHPPPQILIKNTPVPADPPTTFSQAKLASFITQYSTGQVSVEIEWELQKENSKRWN |
| | PEIQYTSNYYKSTNVDFAVNTEGTYSEPRPIGTRYLTRSL |
| SEQ ID | MAADGYLPDWLEDNLSEGIREWWDLKPGAPKPKANQQKQDDGRGLVLPGYKYLGPFNGLDK |
| NO: 117 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPIDSPDSSTGIGKKGQQPAKKKLNFGQTGDSESVPDPQPLGEPP |
| rh.35 | AAPSSVGSGTMAAGGGAPMADNNEGADGVGNASGNWHCDSTWLGDRVITTSTRTWALPTY |
| (AAO881 | NNHLYKQISSSSSGATNDNHYFGYSTPWGYFDFNRFHCHFSPRDWQRLINNNWGFRPKKLRFK |
| 86)) | LFNIQVKEVTTNDGVTTIANNLTSTVQVFSDSEYQLPYVLGSAHQGCLPPFPADVFMIPQYGYLTL |
| | NNGSQSVGRSSFYCLEYFPSQMLRTGNNFEFSYSFEDVPFHSSYAHSQSLDRLMNPLIDQYLYYLA |
| | RTQSTTGSTRELQFHQAGPNTMAEQSKNWLPGPCYRQQGLSKNLDFNNNSNFAWTAATKYHL |
| | NGRNSLTNPGIPMATNKDDEDQFFPINGVLVFGKTGAANKTTLENVLMTSEEEIKTTNPVATEEY |
| | GVVSSNLQPSTAGPQSQTINSQGALPGMVWQNRDVYLQGPIWAKIPHTDGNFHPSPLMGGF |
| | GLKHPPPQILIKNTPVPANPPEVFTPAKFASFITQYSTGQVSVEIEWELQKENSKRWNPEIQYTSN |
| | YAKSNNVEFAVNPDGVYTEPRPIGTRYLPRNL |
| SEQ ID | MAADGYLPDWLEDNLSEGIREWWDLKPGAPKPKANQQKQDDGRGLVLPGYKYLGPFNGLDK |
| NO: 118 | GEPVNAADAAALEHDKAYDQQLEAGDNPYLRYNHADAEFQERLQEDTSFGGNLGRAVFQAKK |
| (AAV | RVLEPLGLVEEGAKTAPGKKRPVEQSPQEPDSSSGIGKKGQQPARKRLNFGQTGDSESVPDPQPL |
| rh.43 | GEPPAAPSGVGPNTMAAGGGAPMADNNEGADGVGSSSGNWHCDSTWLGDRVITTSTRTWA |
| (AAS9924 | LPTYNNHLYKQISNGTSGGATNDNTYFGYSTPWGYFDFNRFHCHFSPRDWQRLINNNWGFRPK |
| 5)) | RLSFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFQFTYTFEDVPFHSSYAHSQSLDRLMNPLIDQ |
| | YLYYLSRTQTTGGTANTQTLGFSQGGPNTMANQAKNWLPGPCYRQQRVSTTTGQNNNSNFA |
| | WTAGTKYHLNGRNSLANPGIAMATHKDDEERFFPVTGSCFWQQNAARDNADYSDVMLTSEEE |
| | IKTTNPVATEEYGIVADNLQQQNTAPQIGTVNSQGALPGMVWQNRDVYLQGPIWAKIPHTDG |
| | NFHPSPLMGGFGLKHPPPQILIKNTPVPADPPTTFNQSKLNSFITQYSTGQVSVEIEWELQKENSK |
| | RWNPEIQYTSNYYKSTSVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 119 | GEPVNAADAAALEHDKAYDQQLKAGDNPYLRYNHADAEFQERLQEDTSFGGNLGRAVFQAKK |
| (AAV | RVLEPLGLVEEAAKTAPGKKRPVEPSPQRSPDSSTGIGKKGQQPARKRLNFGQTGDSESVPDPQP |
| rh.48 | IGEPPAGPSGLGSGTMAAGGGAPMADNNKGADGVGNASGNWHCDSTWLGDRVITTSTRTW |
| (AAS9924 | ALPTYNNHLYKQISSQSAGSTNDNVYFGYSTPWGYFDFNRFHCHFSPRDWQRLINSNWGFRPK |
| 6)) | KLNFKLFNIQVKEVTTNDGVTTIANNLTSTVQVFSDSEYQLPYVLGSAHQGCLPPFPADVFMIPQY |
| | GYLTLNNGSQSVGRSSFYCLEYFPSQMLRTGNNFTFSYTFEDVPFHSSYAHSQSLDRLMNPLIDQ |
| | YLYYLARTQSNAGGTAGNRELQFYQGGPTTMAEQAKNWLPGPCFRQQRVSKTLDQNNNSNFA |
| | WTGATKYHLNGRNSLVNPGVAMATHKDDEERFFPSSGVLIFGKTGAANKTTLENVLMTNEEEIR |
| | PTNPVATEEYGTVSSNLQAANTAAQTQVVNNQGALPGMVWQNRDVYLQGPIWAKIPHTDGN |
| | FHPSPLMGGFGLKHPPPQILIKNTPVPANPPEVFTPAKFASFITQYSTGQVSVEIEWELQKENSKR |
| | WNPEIQYTSNFDKQTGVDFAVDSQGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 120 | GEPVNAADAAALEHDKAYDQQLKAGDNPHLRYNHADAEFQERLQEDTSFGGNLGRAVFQAKK |
| (AAV | RVLEPLGLVEEGAKTAPGKKRPVEPSPQRSPDSSTGIGKKGQQPARKRLNFGQTGDSESVPDPQL |
| rh.49 | IGEPPAAPSSVGSGTMAAGGGAPMADNNEGADGVGSSSGNWHCDSTWLGDRVITTSTRTWA |
| (AAS9924 | LPTYNNHLYKQISNGTSGGSTNDNTYFGYSTPWGYFDFNRFHCHFSPRDWQRLINNNWGFRPK |
| 7)) | RLSFKLFNIQVKEVTQNEGTKTIANNLTSTIQVFTDSEYQLPYVLGSAHQGCLPPFPADVFMIPQY |
| | GNLTLNNGSQAVGRSSFYCLEYFPSQMLRTGNNFSFSYTFEDVPFHSSYAHSQSLDRLMNPLIDQ |
| | YLYYLSRTQSTGGTAGTQQLLFSQAGPSNMSAQARNWLPGPCYRQQRVSTTLSQNNNSNFAW |
| | TGATKYHLNGRDSLVNPGVAMATNKDDEDRFFPSSGILMFGKQGAGKDNMGYSNVMLTSEEE |
| | IKTTNPVATEQYGVVADNLQQQNTAPIVGAVNSQGALPGMVWQNRDVYLQGPIWAKIPHTD |
| | GNFHPSPLMGGFGLKHPPPQILIKNTPVPADPPTAFNQAKLNSFITQYGTGQVSVEIEWELQKEN |
| | SKRWNPEIQYTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 121 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPVEPSPQRSPDSSTGIGKKGQQPAGKRLNFGQTGDSESVPDPQ |
| rh.50 | PIGEPPAAPSSVGSGTMAAGGGAPMADNNEGADGVGSSSGNWHCDSTWLGDRVITTSTRTW |
| (AAS9924 | ALPTYNNHLYKQISNGTSGGSTNDNTYFGYSTPWGYFDFNRFHCHFSPRDWQRLINNNWGFRP |
| 8)) | KRLSFKLFNIQVKEVTQNEGTKTIANNLTSTIQVFTDSEYQLPYVLGSAHQGCLPPFPADVFMIPQ |
| | YGYLTLNNGSQAVGRSSFYCLEYFPSQMLRTGNNFSFSYTFEDVPFHSSYAHSQSLDRLMNPLVD |
| | QYLYYLSRTQSTGGTAGTQQLLFSQAGPSNMSAQARNWLPGPCYRQQRVSTTLSQNNNSNFA |
| | WTGATKYHLNGRDSLVNPGVAMATNKDDEDRFFPSSGILMFGKQGAGKDNVDYSNVMLTSEE |
| | EIKTTNPVATEQYGVVADNLQQQNTAPIVGAVNSQGALPGMVWQNRDVYLQGPIWAKIPHTD |
| | GNFHPSPLMGGFGLKHPPPQILIKNTPVPADPPTAFNQAKLNSFITQYSTGQVSVEIEWELQKEN |
| | SKRWSPEIQYTSNYYKSTNVDFAVNTEGVYSEPRPIGTRYLTRNL |
| SEQ ID | MVADGYLPDWLEDNLSEGIREWWDLKPGAPKPKANQQKQGDGRGLVLPGYKYLGPFNGLDK |
| NO: 122 | GEPVNAADAAALEHDKAYDQQLKAGDNPYLRYNHADAELQERLQEDTSFGGNLGRAVFQAKK |
| (AAV | RVLEPLGLVEEGAKTAPGKKRPVEPSPQRSPDSSTGIGKKGQQPARKRLNFGQTGDSESVPDPQP |
| rh.51 | IGEPPAAPSSVGSGTMAAGGGAPMADNNEGADGVGSSSGNWHCDSTWLGDRVITTSTRTWA |
| (AAS9924 | LPTYNNHLYKQISNGTSGGSTNDNTYFGYSTPWGYFDFNRFHCHFSPRDWQRLINNNWGFRPK |
| 9)) | RLSFKLFNIQVKEVTQNEGTKTIANNLTSTIQVFTDSEYQLPYVLGSAHQGCQPPFPADVFMIPQY |
| | GYLTLNNGSQAVGRSSFYCLEYFPSQMLRTGNNFSFSYTFEDVPFHSSYAHSQSLDRLMNPLIDQ |
| | YLYYLSRTQSTGGTAGTQQLLFSQAGPSNMSAQARNWLPGPCYRQQRVSTTLSQNNNSNFAW |
| | TGATKYHLNGRDSLVNPGVAMATNKDDEDRFFPSSGILMFGKQGAGKDNVDYSNVMLTSEEEI |
| | KTTNPVATEQYGVVADNLQQQNTAPIVGAVNSQGALPGMVWQNRDVYLQGPIWAKIPHTDG |
| | NFHPSPLMGGFGLKHPPPQILIKNTPVPADPPTAFNQAKLNSFITQYSTGQVSVEIEWEPQKENS |
| | KRWNPEIQYTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 123 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPVEPSPQRSPDSSTGIGKKGQQPARKRLNFGQTGDSESVPDPQP |
| rh.52 | IGEPPAAPSSVGSGTMAAGGGAPMADNNEGADGVGSSSGNWHCDSTWLGDRVITTSTRTWA |
| (AAS9925 | LPTYNNHLYKQISNGTSGGSTNDNTYFGYSTPWGYFDFNRFHCHFSPRDWQRLINNNWGFRPK |
| 0)) | RLSFKLFNIQVKEVTQNEGTKTIANSLTSTIQVFTDSEYQLPYVLGSAHQGCLPPFPADVFMIPQYG |
| | YLTPNNGSQAVGRSSFYCLEYFPSQMLRTGNNFSFSYTFEDVPFHSSYAHSQSLDRLMNPLIDQY |
| | LYYLSRTQSTGGTAGTQQLLSSQAGPSNMSAQARNWLPGPCYRQQRVSTTLSQNNNSNFAWT |
| | GATKYHLNGRDSLVNPGVAMATNKDDEDRFFPSSGILMFGKQGAGKDNVDYSNVMLTSEEEIK |
| | TTNPVATEQYGVVADNLQQQNTAPIVGAVNSQGALPGMVWQNRDVYLQGPIWAKIPHTDGN |
| | FHPSPLMGGFGLKHPPPQILIKNTPVPADPPTAFNQAKLNSFITQYSTGQVSVEIEWELQKENSKR |
| | WNPEIQYTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 124 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPVEPSPQRSPDSSTGIGKKGQQPARKRLNFGQTGDSESVPDPQP |
| rh.53 | IGEPPAAPSSVGSGTMAAGGGAPMADNNEGADGVGSSSGNWHCDSTWLGDRVITTSTRTWA |
| (AAS9925 | LPTYNNHLYKQISNGTSGGSTNDNTYFGYSTPWGYFDFNRFHCHFSPRDWQRLINNNWGFRPK |
| 1)) | RLSFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFSFSYTFEDVPFHSSYVHSQSLDRLMNPLIDQ |
| | YLYYLSRTQSTGGTAGTQQLLFSQAGPSNMSAQARNWLPGPCYRQQRVSTTLSQNNNSNFAW |
| | TGATKYHLNGRDSLVNSGVAMATNKDDEDRFFPSSGILMFGKQGAGKDNVDYSNVMLTSEEEI |
| | KTTNPVATEQYGVVADNLQQQNTAPIVGAVNSQGALPGMVWQNRDVYLQGPIWAKIPHTDG |
| | NFHPSPLMGGFGLKHPPPQILIKNTPVPADPPTAFNQAKLNSFITQYSTGQVSVEIEWELQKENS |
| | KRWNPEIQYTSNYYKSTNVDFAVNTEGVYSEPRPIGTRYPTRNL |
| SEQ ID | MAADGYLPDWLEDNLSEGIREWWDLKPGAPKPKANQQKQDDGRGLVLPGYKYLGPFNGLDK |
| NO: 125 | GEPVNAADAAALEHDKAYDQQLKAGDNPYLRYNHADAEFQERLQEDTSFGGNLGRAVFQAKK |
| (AAV | RVLEPLGLVEEAAKTAPGKKRPVEPSPQRSPDSSTGIGKKGQQPARKRLNFGQTGDSESVPDPQP |
| rh.54 | LGEPPAGPSGLGSGTMAAGGGAPMADNNEGADGVGNASGNWHCDSTWLGDRVITTSTRTW |
| (AAS9925 | ALPTYNNHLYKQISSQSAGSTNDNVYFGYSTPWGYFDFNRFHCHFSPRDWQRLINNNWGFRPK |
| 2)) | KLNFKLFNIQVKEVTTNDGVTTIANNLTSTVQVFSDSEYQLPYVLGSAHQGCLPPFPADVFMIPQY |
| | GYLTLNNGSQSVGRSSFYCLEYFPSQVLRTGNNFTFSYTFEDVPFHSSYAHSQSLDRLMNPLIDQY |
| | LYYLARTQSNPGGTSGNRELQFYQGGPSTMAEQAKNWLPGPCFRQQRVSKTLDQNNNSNFA |
| | WTGATKYHLNGRNSLVNPGVAMATHKDDEDRFFPSSGVLIFGKTGATNKTTLENVLMTNEEEIR |
| | PTNPVATEEYGIVSSNLQAANTAAQTQVVNNQGALPGMVWQNRDVYLQGPIWAKIPHTDGN |
| | FHPSPLMGGFGLKHPPPQILIKNTPVPANPPEVFTPAKFASFITQYSTGQVSVEIEWELQKENSKR |
| | WNPEIQYTSNFDKQTGVDFAVDSQGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 126 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPVEPSPQRSPDSSTGIGKKGQQPARKRLNFGQTGDSESVPDPQP |
| rh.55 | IGEPPAAPSSVGSGTMAAGGGAPMADNNEGADGVGSSSGNWHCDSTRLGDRVITTSTRTWAL |
| (AAS9925 | PTYNNHLYKQISSQSAGSTNDNVYFGYSTPWGYFDFNRFHCHFSPRDWQRLINNNWGFRPKKL |
| 3)) | NFKLFNIQVKEVTTNDGVTTIANNLTSTVQVFSDSEYQLPYVLGSAHQGCLPPFPADVFMIPQYG |
| | YLTLNNGSQSVGRSSFYCLEYFPSQMLRTGNNFTFSYTFEDVPFHSSYAHSQSLDRLMNPLIDQYL |
| | YYLARTQSNAGGTAGNRELQFYQGGPTTMAEQAKNWLPGPCFRQRRVSKTLDQNNNSNFAW |
| | TGATKYHLNGRNSLVNPGVAMATHKDDEERFFPSSGVLIFGKTGAANKTTLENVLMTNEEEIRPT |
| | NPVATEEYGTVSSNLQAANTAAQTQVVNNQGALPGMVWQNRDVYLQGPIWAKIPHTDGNFH |
| | PSPLMGGFGLKHPPPQILIKNTPVPANPPEVFTPAKFASFITQYSTGQVSVEIEWELQKENSKRW |
| | NPEIQYTSNFDKQTGVDFAVDSQGVYSEPRPIGTRYLTRNL |
| SEQ ID | MAADGYLPDWLEDNLSEGIREWWALKPGAPKPKANQQKQDDGRGLVLPGYKYLGPFNGLDK |
| NO: 127 | GEPVNAADAAALEHDKAYDQQLQAGDNPYLRYNHADAEFQERLQEDTSFGGNLGRAVFQAKK |
| (AAV | RVLEPLGLVEEGAKTAPGKKRPVEPSPQRSPDSSTGIGKKGQQPARKRLNFGQTGDSESVPDPQP |
| rh.57 | IGEPPAAPSSVGSGTMAAGGGAPMADNNEGADGVGSSSGNWHCDSTWLGDRVITTSTRTWA |
| (AAS9925 | LPTYNNHLYKQTSNGTSGGSTNDNTYFGYSTPWGYFDFNRFHCHFSPRDWQRLINNNWGFRP |
| 4)) | KRLSFKLFNIQVKEVTQNEGTKTIANNLTSTIQVFTDSEYQLPYVLGSAHQGCLPPFPADVFMIPQ |
| | YGYLTLNNGSQAVGRSSFYCLEYFPSQMLRTGNNFSFSYTFEDVPFHSSYAHSQSLDRLMNPLID |
| | QYLYYLSRTQSTGGTAGTQQLLFSQAGPSNMSAQARNWLPGPCYRQQRVSTTLSQNNNSNFA |
| | WTGATKYHLNGRDSLVNPGVAMATNKDDEDRFFPSSGILMFGKQGAGKDNVDYSNVMLTSEE |
| | EIKTTNPVATEQYGVVADNLQQQNTAPIVGAVNSQGALPGMVWQNRDVYLQGPIWAKIPHTD |
| | GNFHPSPLMGGFGLKHPPPQILIKNTPVPADPPTAFNQAKLNSFITQYSTGQVSAEIEWELQKEN |
| | SKRWNPEIQYTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 128 | GEPVNAADAAALEHDKAYDQQLKAGDNPYLRYNHADAEFQERLQEDTSFGGNLGRAVFQAKK |
| (AAV | RVLEPLGLVEEAAKTAPGKKRPVEPSPQRSPDSSTGIGKKGQQPARKRLNFGQTGDSESVPDPQP |
| rh.58 | IGEPPAAPSSVGSGTMAAGGGAPMADNNEGADGVGSSSGNWHCDSTWLGDRVITTSTRTWA |
| (AAS9925 | LPTYNNHLYKQISNGTSGGSTNDNTYFGYSTPWGYFDFNRFHCHFSPRDWQRLINNNWGFRPK |
| 5)) | RLSFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFSFSYTFEDVPFHSSYAHSQSLDRLMNPLIDQ |
| | YLYYLSRTQSTGGTAGTQQLLFSQAGPSNMSAQARNWLPGPCYRQQRVSTTLSQNNNSNFAW |
| | TGATKYHLNGRDSLVNPGVAMATNKDDEDRFFPSSGILMFGKQGAGKDNVDYSNVMLTSEEEI |
| | KTTNPVATEQYGVVADNLQQQNTAPIVGAVNSQGALPGMVWQNRDVYLQGPIWAKIPHTDG |
| | NFHPSPLMGGFGLKHPPPQILIKSTPVPADPPTAFNQAKLNSFITQYSTGQVSVEIEWELQKENSK |
| | CWNPEIQYTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 129 | GEPVNAADAAALEHDKAYDQQLKAGDNPYLRYNHADAEFQERLQEDTSFGGNLGRAVFQAKK |
| (AAV | RVLEPLGLAEEAAKTAPGKKRPVEPSPQRSPDSSTGIGKKGQQPARKRLNFGQTGDSESVPDPQP |
| rh.62 | IGEPPAGPSGLGSGTMAAGGGAPMADNNKGADGVGNASGNWHCDSTWLGDRVITTSTRTW |
| (AAS9925 | ALPTYNNHLYKQISSQSAGSTNDNVYFGYSTPWGYFDFNRFHCHFSPRDWQRLINNNWGFRPK |
| 8)) | KLNFKLFNIQVKEVTTGDGVTTIANNLTSTVQVFSDSEYQLPYVLGSAHQGCLPPFPADVFMIPQY |
| | GYLTLNNDSQSVGRSSFYCLEYFPSQMLRTGNNFTFSYTFEDVPFHSSYAHSQSLDRLMNPLIDQ |
| | YLYYLARTQSNAGGTAGNRELQFYQGGPTTMAEQAKNWLPGPCFRQQRVSKTLDQNNNSNFA |
| | WTGATKYHLNGRNSLVNPGVAMATHKDDEERFFPSSGVLIFGKTGAANKTTLENVLMTNEEEIR |
| | PTNPVATEEYGTVSSNLQAANTAAQTQVVNNQGALPGMVWQNRDVYLQGPIWAKIPHTDGN |
| | FHPSPLMGGFGLKHPPPQILIKNTPVPANPPEVFTPAKFASFITQYSTGQVSVEIEWELQKENSKR |
| | WNPEIQYTSNFDKQTGVDFAVDSQGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 130 | GEPVNAADAAALEHDKAYDQQLKAGDNPYLRYNHADAEFQERLQEDTSFGGNLGRAVFQAKK |
| (AAV | RVLEPLGLVEEGAKTAPGKKRPVEPSPQRSPDSSTGIGKKGQQPARKRLNFGQTGDSESVPDPQP |
| rh.64 | IGEPPAAPSSVGSGTMAAGGGAPMADNNEGADGVGSSSGNWHCDSTWLGDRVITTSTRTWA |
| (AAS9925 | LPTYNNHLYKQISNGTSGGSTNDNTYFGYSTPWGYFDFNRFHCHFSPRDWQRLINNNWGFRPK |
| 9)) | RLSFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFSFSYTFEDVPFHSSYAHSQSLDRLMNPLIDQ |
| | YLYYLSRTQSTGGTAGTQQLLFSQAGPSNMSAQARNWLPGPCYRQQRVSTTLSQNNNSNFAW |
| | TGATKYHLNGRDSLVNPGVAMATNKDDEDRFFPSSGILMFGKQGAGKDNVDYSNVMLTSEEEI |
| | KTTNPVATEQYGVVADNLQQQNTAPIVGAVNSQGALPGMVWQNRDVYLQGPIWAKIPHTDG |
| | NFHPSPLMGGFGLKHPPPQILIKNTPVPADPPTAFNQAKLNSFITQYSTGQVSVEIVWELQKENS |
| | KRRNPEIQYTSNYYKSTNVDFAVNTEGVYSEPRPIGTRYLTRNL |
| SEQ ID | MAADGYLPDWLEDTLSEGIRQWWKLKPGPPPPKPAERHKDDSRGLVLPGYKYLGPFNGLDKGE |
| NO: 131 | PVNEADAAALEHDKAYDRQLDSGDNPYLKYNHADAEFQERLKEDTSFGGNLGRAVFQAKKRVL |
| (AAV | EPLGLVEEPVKTAPGKKRPVEHSPAEPDSSSGTGKAGQQPARKRLNFGQTGDADSVPDPQPLG |
| rh.56 | QPPAAPSGLGSTTMATGSGAPMADNNEGADGVGNSSGNWHCDSQWLGDRVITTSTRTWAQ |
| (JA40016 | PTYNNHLYKQISSQSGASNDNHYFGYSTPWGYFDFNRFHCHFSPRDWQRLINNNWGFRPKRLN |
| 4)) | FKLFNIQVKEVTQNDGTTTIANNLTSTVQVFTDSEYQLPYVLGSAHQGCLPPFPADVFMVPQYGY |
| | LTLNNGSQAVGRSSFYCLEYFPSQMLRTGNNFQFSYTFEDVPFHSSYAHSQSLDRLMNPLIDQYL |
| | YYLNKTQSNSGALQQSRLLFSQAGPTSMSLQAKNWLPGPCYRQQRLSKQANDNNNSNFPWTA |
| | ATKYHLNGRDSLVNPGPAMASHKDDEEKFFPMHGTLIFGKQGTNANDADLDNVMITDEEEIRT |
| | TNPVATEQYGYVSNNLQNSNTGPTTGTVNHRGALPGMVWQDRDVYLQGPIWAKIPHTDGHF |
| | HPSPLMGGFGLKHPPPQIMIKNTPVPANPPTNFSSAKFASFITQYSTGQVSVEIEWELQKENSKR |
| | WNPEIQYTSNYNKSVNVDFTVDTNGVYSEPRPIGTRYLTRNL |
| SEQ ID | MAADGYLPDWLEDNLSEGIREWWDLKPGAPKPKANQQKQDDGRGLVLPGYKYLGPFNGLD |
| NO: 132 | KGEPVNAADAAALEHDKAYDQQLKAGDNPYLRYNHADAEFQERLQEDTSFGGNLGRAVFQ |
| (Anc80) | AKKRVLEPLGLVEEGAKTAPGKKRPVEQSPQEPDSSSGIGKKGQQPAXKRLNFGQTGDSE |
| | SVPDPQPLGEPPAAPSGVGSNTMAXGGGAPMADNNEGADGVGNASGNWHCDSTWLGDRVI |
| | TTSTRTWALPTYNNHLYKQISSQSGXSTNDNTYFGYSTPWGYFDFNRFHCHFSPRDWQRL |
| | INNNWGFRPKXLNFKLFNIQVKEVTTNDGTTTIANNLTSTVQVFTDSEYQLPYVLGSAHQ |
| | GCLPPFPADVFMIPQYGYLTLNNGSQAVGRSSFYCLEYFPSQMLRTGNNFXFSYTFEDVP |
| | FHSSYAHSQSLDRLMNPLIDQYLYYLSRTQTTSGTAGNRXLQFSQAGPSSMANQAKNWLP |
| | GPCYRQQRVSKTXNQNNNSNFAWTGATKYHLNGRDSLVNPGPAMATHKDDEDKFFPMSGV |
| | LIFGKQGAGNSNVDLDNVMITXEEEIKTTNPVATEXYGTVATNLQSXNTAPATGTVNSQG |
| | ALPGMVWQXRDVYLQGPIWAKIPHTDGHFHPSPLMGGFGLKHPPPQILIKNTPVPANPPT |
| | TFSPAKFASFITQYSTGQVSVEIEWELQKENSKRWNPEIQYTSNYNKSTNVDFAVDTNGV |
| | YSEPRPIGTRYLTRNL |
| | (168) . . . (168) Lys or Arg |
| | (205) . . . (205) Ala or Ser |
| | (266) . . . (266) Ala or Gly |
| | (311) . . . (311) Arg or Lys |
| | (411) . . . (411) Glu or Gln |
| | (460) . . . (460) Thr or Glu |
| | (493) . . . (493) Ala or Thr |
| | (562) . .. (562) Ser or Asn |
| | (576) . . . (576) Gln or Glu |
| | (587) . . . (587) Ser or Ala |
| | (609) . . . (609) Asn or Asp |
| SEQ ID | MAADGYLPDWLEDNLSEGIREWWDLKPGAPKPKANQQKQDDGRGLVLPGYKYLGPFNGLD |
| NO: 133 | KGEPVNAADAAALEHDKAYDQQLKAGDNPYLRYNHADAEFQERLQEDTSFGGNLGRAVFQ |
| (Anc81 | AKKRVLEPLGLVEEGAKTAPGKKRPVEQSPQEPDSSXGIGKKGQQPAXKRLNFGQTGDSE |
| (AKU895 | SVPDPQPLGEPPAAPSGVGSNTMAAGGGAPMADNNEGADGVGNASGNWHCDSTWLGDRVI |
| 96)) | TTSTRTWALPTYNNHLYKQISXXQSGGSTNDNTYFGYSTPWGYFDFNRFHCHFSPRDWQR |
| | LINNNWGFRPKXLNFKLFNIQVKEVTTNDGTTTIANNLTSTVQVFTDSEYQLPYVLGSAH |
| | QGCLPPFPADVFMIPQYGYLTLNNGSQAVGRSSFYCLEYFPSQMLRTGNNFXFSYTFEDV |
| | PFHSSYAHSQSLDRLMNPLIDQYLYYLSRTQTTGGTAGNXXLQFSQAGPSSMANQAKNWL |
| | PGPCYRQQRVSKTTNQNNNSNFAWTGATKYHLNGRDSLVNPGVAMATHKDDEDRFFPSSG |
| | VLIFGKQGAGNXNVDXXNVMITXEEEIKTTNPVATEEYGXVATNLQSXNTAPQTGTVNSQ |
| | GALPGMVWQNRDVYLQGPIWAKIPHTDGNFHPSPLMGGFGLKHPPPQILIKNTPVPANPP |
| | TTFXPAKFASFITQYSTGQVSVEIEWELQKENSKRWNPEIQYTSNYNKSTNVDFAVDTEG |
| | VYSEPRPIGTRYLTRNL |
| | (157) . . . (157) Thr or Ser |
| | (168) . . . (168) Lys or Arg |
| | (262) . . . (262) Asn or Ser |
| | (263) . . . (263) Ser or His |
| | (312) . . . (312) Arg or Lys |
| | (412) . . . (412) Glu or Gln |
| | (460) . . . (460) Arg or Gln |
| | (461) . . . (461) Thr or Glu |
| | (552) . . . (552) Asp or Ser |
| | (556) . . . (556) Leu or Tyr |
| | (557) . . . (557) Asp or Ser |
| | (563) . . . (563) Ser or Asn |
| | (580) . . . (580) Val or Ile |
| | (588) . . . (588) Asn or Ser |
| | (664) . . . (664) Ser or Thr |
| SEQ ID | MAADGYLPDWLEDNLSEGIREWWDLKPGAPKPKANQQKQDDGRGLVLPGYKYLGPFNGLD |
| NO: 134 | KGEPVNAADAAALEHDKAYDQQLKAGDNPYLRYNHADAEFQERLQEDTSFGGNLGRAVFQ |
| (Anc82 | AKKRVLEPLGLVEEGAKTAPGKKRPVEQSPQREPDSSXGIGKKGQQPAXKRLNFGQTGDS |
| (AKU895 | ESVPDPQPLGEPPAAPSGVGSNTMAAGGGAPMADNNEGADGVGNSSGNWHCDSTWLGDR |
| 97)) | V ITTSTRTWALPTYNNHLYKQISNGTSGGSTNDNTYFGYSTPWGYFDFNRFHCHFSPRDWQ |
| | RLINNNWGFRPKRLNFKLFNIQVKEVTTNEGTKTIANNLTSTVQVFTDSEYQLPYVLGSA |
| | HQGCLPPFPADVFMIPQYGYLTLNNGSQAVGRSSFYCLEYFPSQMLRTGNNFQFSYTFED |
| | VPFHSSYAHSQSLDRLMNPLIDQYLYYLSRTQTTGGTAGTQTLQFSQAGPSSMANQAKNW |
| | LPGPCYRQQRVSTTTNQNNNSNFAWTGATKYHLNGRDSLVNPGVAMATHKDDEDRFFPSS |
| | GVLIFGKQGAGNDNVDYSNVMITXEEEIKTTNPVATEEYGVVATNLQSANTAPQTGTVNS |
| | QGALPGMVWQNRDVYLQGPIWAKIPHTDGNFHPSPLMGGFGLKHPPPQILIKNTPVPADP |
| | PTTFNQAKLNSFITQYSTGQVSVEIEWELQKENSKRWNPEIQYTSNYYKSTNVDFAVNTE |
| | GVYSEPRPIGTRYLTRNL |
| | (158) . . . (158) Thr or Ser |
| | (169) . . . (169) Lys or Arg |
| | (564) . . . (564) Ser or Asn |
| SEQ ID | MAADGYLPDWLEDNLSEGIREWWDLKPGAPKPKANQQKQDDGRGLVLPGYKYLGPFNGLD |
| NO: 135 | KGEPVNAADAAALEHDKAYDQQLKAGDNPYLRYNHADAEFQERLQEDTSFGGNLGRAVFQ |
| (Anc83 | AKKRVLEPLGLVEEGAKTAPGKKRPVEQSPQREPDSSXGIGKKGQQPAXKRLNFGQTGDS |
| (AKU895 | ESVPDPQPLGEPPAAPSGVGSNTMAAGGGAPMADNNEGADGVGSSSGNWHCDSTWLGDRV |
| 98)) | ITTSTRTWALPTYNNHLYKQISNGTSGGSTNDNTYFGYSTPWGYFDFNRFHCHFSPRDWQ |
| | RLINNNWGFRPKRLXFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFXFSYTFED |
| | VPFHSSYAHSQSLDRLMNPLIDQYLYYLSRTQTTGGTAGTQTLQFSQAGPSXMANQAKNW |
| | LPGPCYRQQRVSTTTSQNNNSNFAWTGATKYHLNGRDSLVNPGVAMATHKDDEXRFFPSS |
| | GXLIFGKQGAGKDNVDYSNVMLTSEEEIKTTNPVATEEYGVVADNLQQQNTAPQXGTVNS |
| | QGALPGMVWQNRDVYLQGPIWAKIPHTDGNFHPSPLMGGFGLKHPPPQILIKNTPVPADP |
| | PTTFNQAKLNSFITQYSTGQVSVEIEWELQKENSKRWNPEIQYTSNYYKSTNVDFAVNTE |
| | GVYSEPRPIGTRYLTRNL |
| | (158) . . . (158) Thr or Ser |
| | (169) . . . (169) Arg or Lys |
| | (315) . . . (315) Asn or Ser |
| | (413) . . . (413) Gln or Glu |
| | (472) . . . (472) Asn, Thr or Ser |
| | (534) . . . (534) Asp or Glu |
| | (542) . . . (542) Ile or Val |
| | (595) . . . (595) Ile or Val |
| SEQ ID | MAADGYLPDWLEDNLSEGIREWWDLKPGAPKPKANQQKQDDGRGLVLPGYKYLGPFNGLD |
| NO: 136 | KGEPVNAADAAALEHDKAYDQQLKAGDNPYLRYNHADAEFQERLQEDTSFGGNLGRAVFQ |
| (Anc84 | AKKRVLEPLGLVEEGAKTAPGKKRPVEPSPQRSPDSSTGIGKKGQQPAXKRLNFGQTGDS |
| (AKU895 | ESVPDPQPIGEPPAAPSGVGSGTMAAGGGAPMADNNEGADGVGSSSGNWHCDSTWLGDRV |
| 99)) | ITTSTRTWALPTYNNHLYKQISNGTSGGSTNDNTYFGYSTPWGYFDFNRFHCHFSPRDWQ |
| | RLINNNWGFRPKRLXFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTAGTQQLLFSQAGPSNMSAQAKNW |
| | LPGPCYRQQRVSTTLSQNNNSNFAWTGATKYHLNGRDSLVNPGVAMATHKDDEXRFFPSS |
| | GXLMFGKQGAGKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTTFNQAKLNSFITQYSTGQVSVEIEWELQKENSKRWNPEIQYTSNYYKSTNVDFAVNTE |
| | GVYSEPRPIGTRYLTRNL |
| | (169) . . . (169) Arg or Lys |
| | (315) . . . (315) Asn or Ser |
| | (534) . . . (534) Asp or Glu |
| | (542) . . . (542) Ile or Val |
| SEQ ID | MAADGYLPDWLEDNLSEGIREWWDLKPGAPKPKANQQKQDDGRGLVLPGYKYLGPFNGLD |
| NO: 137 | KGEPVNAADAAALEHDKAYDQQLKAGDNPYLRYNHADAEFQERLQEDTSFGGNLGRAVFQ |
| (Anc94) | AKKRVLEPLGLVEEGAKTAPGKKRPVEPSPQRSPDSSTGIGKKGQQPAKKRLNFGQTGDS |
| | ESVPDPQPIGEPPAGPSGLGSGTMAAGGGAPMADNNEGADGVGSSSGNWHCDSTWLGDRV |
| | ITTSTRTWALPTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSEYQLPYVLGSA |
| | HQGCLPPFPADVFMIPQYGYLTLNNGSQAVGRSSFYCLEYFPSQMLRTGNNFEFSYTFED |
| | VPFHSSYAHSQSLDRLMNPLIDQYLYYLSRTQSTGGTAGTQQLLFSQAGPXNMSAQAKNW |
| | LPGPCYRQQRVSTTLSQNNNSNFAWTGATKYHLNGRDSLVNPGVAMATHKDDEERFFPSS |
| | GVLMFGKQGAGKDNVDYSSVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQILIKNTPVPADP |
| | PTTFSQAKLASFITQYSTGQVSVEIEWELQKENSKRWNPEIQYTSNYYKSTNVDFAVNTE |
| | GTYSEPRPIGTRYLTRNL |
| | (471) . . . (471) Ser or Asn |
| SEQ ID | MAADGYLPDWLEDNLSEGIREWWDLKPGAPKPKANQQKQDDGRGLVLPGYKYLGPFNGLD |
| NO: 138 | KGEPVNAADAAALEHDKAYDQQLKAGDNPYLRYNHADAEFQERLQEDTSFGGNLGRAVFQ |
| (Anc110 | AKKRVLEPLGLVEEGAKTAPGKKRPVEQSPQEPDSSXLGIGKTGQQPAXKRLNFGQTGDS |
| (AKU896 | ESVPDPQPLGEPPAAPSGVGSNTMASGGGAPMADNNEGADGVGNSSGNWHCDSTWLGDRV |
| 00)) | ITTSTRTWALPTYNNHLYKQISNGTSGGSTNDNTYFGYSTPWGYFDFNRFHCHFSPRDWQ |
| | RLINNNWGFRPKRLNFKLFNIQVKEVTTNEGTKTIANNLTSTVQVFTDSEYQLPYVLGSA |
| | HQGCLPPFPADVFMIPQYGYLTLNNGSQAVGRSSFYCLEYFPSQMLRTGNNFQFSYTFED |
| | VPFHSSYAHSQSLDRLMNPLIDQYLYYLSRTQTTGTXGTQTLXFSQAGPSSMANQARNWV |
| | PGPCYRQQRVSTTTNQNNNSNFAWTGAXKXXLNGRDSLMNPGVAMASHKDDEDRFFPSSG |
| | VLIFGKQGAGNDNVDYSXVMITNEEEIKTTNPVATEEYGAVATNXQXLANTQAQTGLVHN |
| | QGVLPGMVWQNRDVYLQGPIWAKIPHTDGNFHPSPLMGGFGLKHPPPQILIKNTPVPADP |
| | PTTFNQAKLNSFITQYSTGQVSVEIEWELQKENSKRWNPEIQYTSNYYKSTNVDFAVNTE |
| | GVYSEPRPIGTRYLTRNL |
| | (157) . . . (157) Ser or Thr |
| | (169) . . . (169) Lys or Arg |
| | (457) . . . (457) Ala or Gly |
| | (463) . . . (463) Gln or Ala |
| | (508) . . . (508) Thr or Ala |
| | (510) . . . (510) Tyr or Phe |
| | (511) . . . (511) His or Lys |
| | (558) . . . (558) Gln or Asn |
| | (585) . . . (585) Asn or His |
| | (587) . . . (587) Ser or Ala |
| SEQ ID | MAADGYLPDWLEDNLSEGIREWWDLKPGAPKPKANQQKQDDGRGLVLPGYKYLGPFNGLD |
| NO: 139 | KGEPVNAADAAALEHDKAYDQQLKAGDNPYLRYNHADAEFQERLQEDTSFGGNLGRAVFQ |
| (Anc113 | AKKRVLEPLGLVEEGAKTAPGKKRPVEXSPQRSPDSSTGIGKKGQQPAXKRLNFGQTGDS |
| (AKU896 | ESVPDPQPLGEPPAAPSGVGSGTMAAGGGAPMADNNEGADGVGNASGNWHCDSTWLGDR |
| 01)) | V ITTSTRTWALPTYNNHLYKQISSQSAGSTNDNTYFGYSTPWGYFDFNRFHCHFSPRDWQR |
| | LINNNWGFRPKKLXFKLFNIQVKEVTTNDGVTTIANNLTSTVQVFSDSEYQLPYVLGSAH |
| | QGCLPPFPADVFMIPQYGYLTLNNGSQSVGRSSFYCLEYFPSQMLRTGNNFEFSYTFEDV |
| | PFHSSYAHSQSLDRLMNPLIDQYLYYLARTQSTTGGTAGNRELQFXQAGPSTMAEQAKNW |
| | LPGPCYRQQRVSKTLDQNNNSNFAWTGATKYHLNGRNSLVNPGVAMATHKDDEDRFFPSS |
| | GVLIFGKTGAANKTTLENVLMTXEEEIKTTNPVATEEYGXVSSNLQSXNTAPQTQTVNSQ |
| | GALPGMVWQNRDVYLQGPIWAKIPHTDGNFHPSPLMGGFGLKHPPPQILIKNTPVPANPP |
| | EVFTPAKFASFITQYSTGQVSVEIEWELQKENSKRWNPEIQYTSNYDKSTNVDFAVDSEG |
| | VYSEPRPIGTRYLTRNL |
| | (148) . . . (148) Pro or Gln |
| | (169) . . . (169) Lys or Arg |
| | (314) . . . (314) Arg or Asn |
| | (466) . . . (466) Tyr or His |
| | (563) . . . (563) Asn or Ser |
| | (580) . . . (580) Val or Ile |
| | (588) . . . (588) Ala or Ser |
| SEQ ID | MAADGYLPDWLEDNLSEGIREWWDLKPGAPKPKANQQKQDDGRGLVLPGYKYLGPFNGLD |
| NO: 140 | KGEPVNAADAAALEHDKAYDQQLKAGDNPYLRYNHADAEFQERLQEDTSFGGNLGRAVFQ |
| (Anc126 | AKKRVLEPLGLVEEGAKTAPGKKRPVEQSPQEPDSSSGIGKXGQQPAXKRLNFGQTGDSE |
| (AKU896 | SVPDPQPLGEPPAAPSGVGSNTMASGGGAPMADNNEGADGVGNXSGNWHCDSTWLGDRVI |
| 02)) | TTSTRTWALPTYNNHLYKQISSQSGASNDNHYFGYSTPWGYFDFNRFHCHFSPRDWQRLI |
| | NNNWGFRPKXLNFKLFNIQVKEVTTNDGTTTIANNLTSTVQVFTDSEYQLPYVLGSAHQG |
| | CLPPFPADVFMIPQYGYLTLNNGSQAVGRSSFYCLEYFPSQMLRTGNNFXFSYTFEDVPF |
| | HSSYAHSQSLDRLMNPLIDQYLYYLXRTQTTSGTAQNRELXFSQAGPSSMXNQAKNWLPG |
| | PCYRQQRVSKTANDNNNSNFAWTGATKYHLNGRDSLVNPGPAMASHKDDEDKFFPMSGVL |
| | IFGKQGAGASNVDLDNVMITDEEEIKTTNPVATEQYGTVATNLQSSNTAPATGTVNSQGA |
| | LPGMVWQDRDVYLQGPIWAKIPHTDGHFHPSPLMGGFGLKHPPPQILIKNTPVPANPPTT |
| | FSPAKFASFITQYSTGQVSVEIEWELQKENSKRWNPEIQYTSNYNKSXNVDFTVDTNGVY |
| | SEPRPIGTRYLTRNL |
| | (162) . . . (162) Ser or Thr |
| | (168) . . . (168) Lys or Arg |
| | (224) . . . (224) Ala or Ser |
| | (310) . . . (310) Arg or Lys |
| | (410) . . . (410) Thr or Gln |
| | (446) . . . (446) Ser or Asn |
| | (461) . . . (461) Gln or Leu |
| | (471) . . . (471) Ala or Ser |
| | (708) . . . (708) Ala or Thr |
| SEQ ID | MAADGYLPDWLEDNLSEGIREWWDLKPGAPQPKANQQHQDDXRGLVLPGYKYLGPFNGLD |
| NO: 141 | KGEPVNEADAAALEHDKAYDQQLKAGDNPYLKYNHADAEFQERLQEDTSFGGNLGRAVFQ |
| Anc127 | AKKRVLEPLGLVEEAAKTAPGKKRPVEQSPQEPDSSSGIGKSGQQPAXKRLNFGQTGDSE |
| (AKU896 | SVPDPQPLGEPPAAPSGVGSNTMASGGGAPMADNNEGADGVGNSSGNWHCDSTWLGDRVI |
| 03) | TTSTRTWALPTYNNHLYKQISSQSGASNDNHYFGYSTPWGYFDFNRFHCHFSPRDWQRLI |
| | NNNWGFRPKXLNFKLFNIQVKEVTQNDGTTTIANNLTSTVQVFTDSEYQLPYVLGSAHQG |
| | CLPPFPADVFMIPQYGYLTLNNGSQAVGRSSFYCLEYFPSQMLRTGNNFXFSYTFEDVPF |
| | HSSYAHSQSLDRLMNPLIDQYLYYLXRTQTTSGTTQQSRLXFSQAGPSSMXQQAXNWLPG |
| | PCYRQQRVSKTANDNNNSNFAWTXATKYHLNGRDSLVNPGPAMASHKDDEEKFFPMHGXL |
| | IFGKQGTGASNVDLDNVMITDEEEIRTTNPVATEQYGTVATNLQSSNTAPATGTVNSQGA |
| | LPGMVWQDRDVYLQGPIWAKIPHTDGHFHPSPLMGGFGLKHPPPQILIKNTPVPANPPTT |
| | FSPAKFASFITQYSTGQVSVEIEWELQKENSKRWNPEIQYTSNYNKSVNVDFTVDTNGVY |
| | SEPRPIGTRYLTRNL |
| | (42) . . . (42) Gly or Ser |
| | (168) . . . (168) Arg or Lys |
| | (310) . . . (310) Lys or Arg |
| | (410) . . . (410) Thr or Gln |
| | (446) . . . (446) Ser or Arg |
| | (461) . . . (461) Gln or Leu |
| | (471) . . . (471) Ala or Ser |
| | (475) . . . (475) Lys or Arg |
| | (504) . . . (504) Gly or Ala |
| | (539) . . . (539) Val or Asn |
| SEQ ID | MAADGYLPDWLEDNLSEGIREWWDLKPGAPKPKANQQKQDDGRGLVLPGYKYLGPFNGLDK |
| NO: 142 | GEPVNAADAAALEHDKAYDQQLKAGDNPYLRYNHADAEFQERLQEDTSFGGNLGRAVFQAKK |
| (Anc80L6 | RVLEPLGLVEEGAKTAPGKKRPVEQSPQEPDSSSGIGKKGQQPARKRLNFGQTGDSESVPDPQPL |
| 5 | GEPPAAPSGVGSNTMAAGGGAPMADNNEGADGVGNASGNWHCDSTWLGDRVITTSTRTW |
| (AKU895 | ALPTYNNHLYKQISSQSGGSTNDNTYFGYSTPWGYFDFNRFHCHFSPRDWQRLINNNWGFRPK |
| 95)) | KLNFKLFNIQVKEVTTNDGTTTIANNLTSTVQVFTDSEYQLPYVLGSAHQGCLPPFPADVFMIPQY |
| | GYLTLNNGSQAVGRSSFYCLEYFPSQMLRTGNNFQFSYTFEDVPFHSSYAHSQSLDRLMNPLIDQ |
| | YLYYLSRTQTTSGTAGNRTLQFSQAGPSSMANQAKNWLPGPCYRQQRVSKTTNQNNNSNFAW |
| | TGATKYHLNGRDSLVNPGPAMATHKDDEDKFFPMSGVLIFGKQGAGNSNVDLDNVMITNEEEI |
| | KTTNPVATEEYGTVATNLQSANTAPATGTVNSQGALPGMVWQDRDVYLQGPIWAKIPHTDGH |
| | FHPSPLMGGFGLKHPPPQILIKNTPVPANPPTTFSPAKFASFITQYSTGQVSVEIEWELQKENSKR |
| | WNPEIQYTSNYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 143 | GEPVNAADAAALEHDKAYDQQLKAGDNPYLRYNHADAEFQERLQEDTSFGGNLGRAVFQAKK |
| (Anc80L1) | RVLEPLGLVEEGAKTAPGKKRPVEQSPQEPDSSSGIGKKGQQPAKKRLNFGQTGDSESVPDPQPL |
| | GEPPAAPSGVGSNTMAAGGGAPMADNNEGADGVGNASGNWHCDSTWLGDRVITTSTRTW |
| | ALPTYNNHLYKQISSQSGASTNDNTYFGYSTPWGYFDFNRFHCHFSPRDWQRLINNNWGFRPK |
| | RLNFKLFNIQVKEVTTNDGTTTIANNLTSTVQVFTDSEYQLPYVLGSAHQGCLPPFPADVFMIPQY |
| | GYLTLNNGSQAVGRSSFYCLEYFPSQMLRTGNNFEFSYTFEDVPFHSSYAHSQSLDRLMNPLIDQ |
| | YLYYLSRTQTTSGTAGNRTLQFSQAGPSSMANQAKNWLPGPCYRQQRVSKTANQNNNSNFA |
| | WTGATKYHLNGRDSLVNPGPAMATHKDDEDKFFPMSGVLIFGKQGAGNSNVDLDNVMITSEE |
| | EIKTTNPVATEQYGTVATNLQSSNTAPATGTVNSQGALPGMVWQNRDVYLQGPIWAKIPHTD |
| | GHFHPSPLMGGFGLKHPPPQILIKNTPVPANPPTTFSPAKFASFITQYSTGQVSVEIEWELQKENS |
| | KRWNPEIQYTSNYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 144 | GEPVNAADAAALEHDKAYDQQLKAGDNPYLRYNHADAEFQERLQEDTSFGGNLGRAVFQAKK |
| (Anc80L2 | RVLEPLGLVEEGAKTAPGKKRPVEQSPQEPDSSSGIGKKGQQPARKRLNFGQTGDSESVPDPQPL |
| 7) | GEPPAAPSGVGSNTMAAGGGAPMADNNEGADGVGNASGNWHCDSTWLGDRVITTSTRTW |
| | ALPTYNNHLYKQISSQSGGSTNDNTYFGYSTPWGYFDFNRFHCHFSPRDWQRLINNNWGFRPK |
| | RLNFKLFNIQVKEVTTNDGTTTIANNLTSTVQVFTDSEYQLPYVLGSAHQGCLPPFPADVFMIPQY |
| | GYLTLNNGSQAVGRSSFYCLEYFPSQMLRTGNNFEFSYTFEDVPFHSSYAHSQSLDRLMNPLIDQ |
| | YLYYLSRTQTTSGTAGNRTLQFSQAGPSSMANQAKNWLPGPCYRQQRVSKTANQNNNSNFA |
| | WTGATKYHLNGRDSLVNPGPAMATHKDDEDKFFPMSGVLIFGKQGAGNSNVDLDNVMITNEE |
| | EIKTTNPVATEQYGTVATNLQSANTAPATGTVNSQGALPGMVWQDRDVYLQGPIWAKIPHTD |
| | GHFHPSPLMGGFGLKHPPPQILIKNTPVPANPPTTFSPAKFASFITQYSTGQVSVEIEWELQKENS |
| | KRWNPEIQYTSNYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 145 | GEPVNAADAAALEHDKAYDQQLKAGDNPYLRYNHADAEFQERLQEDTSFGGNLGRAVFQAKK |
| (Anc80L3 | RVLEPLGLVEEGAKTAPGKKRPVEQSPQEPDSSSGIGKKGQQPAKKRLNFGQTGDSESVPDPQPL |
| 3) | GEPPAAPSGVGSNTMAAGGGAPMADNNEGADGVGNASGNWHCDSTWLGDRVITTSTRTW |
| | ALPTYNNHLYKQISSQSGGSTNDNTYFGYSTPWGYFDFNRFHCHFSPRDWQRLINNNWGFRPK |
| | KLNFKLFNIQVKEVTTNDGTTTIANNLTSTVQVFTDSEYQLPYVLGSAHQGCLPPFPADVFMIPQY |
| | GYLTLNNGSQAVGRSSFYCLEYFPSQMLRTGNNFEFSYTFEDVPFHSSYAHSQSLDRLMNPLIDQ |
| | YLYYLSRTQTTSGTAGNRTLQFSQAGPSSMANQAKNWLPGPCYRQQRVSKTANQNNNSNFA |
| | WTGATKYHLNGRDSLVNPGPAMATHKDDEDKFFPMSGVLIFGKQGAGNSNVDLDNVMITSEE |
| | EIKTTNPVATEQYGTVATNLQSSNTAPATGTVNSQGALPGMVWQNRDVYLQGPIWAKIPHTD |
| | GHFHPSPLMGGFGLKHPPPQILIKNTPVPANPPTTFSPAKFASFITQYSTGQVSVEIEWELQKENS |
| | KRWNPEIQYTSNYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 146 | GEPVNAADAAALEHDKAYDQQLKAGDNPYLRYNHADAEFQERLQEDTSFGGNLGRAVFQAKK |
| (Anc80L3 | RVLEPLGLVEEGAKTAPGKKRPVEQSPQEPDSSSGIGKKGQQPAKKRLNFGQTGDSESVPDPQPL |
| 6) | GEPPAAPSGVGSNTMASGGGAPMADNNEGADGVGNASGNWHCDSTWLGDRVITTSTRTWA |
| | LPTYNNHLYKQISSQSGGSTNDNTYFGYSTPWGYFDFNRFHCHFSPRDWQRLINNNWGFRPKK |
| | LNFKLFNIQVKEVTTNDGTTTIANNLTSTVQVFTDSEYQLPYVLGSAHQGCLPPFPADVFMIPQY |
| | GYLTLNNGSQAVGRSSFYCLEYFPSQMLRTGNNFEFSYTFEDVPFHSSYAHSQSLDRLMNPLIDQ |
| | YLYYLSRTQTTSGTAGNRTLQFSQAGPSSMANQAKNWLPGPCYRQQRVSKTANQNNNSNFA |
| | WTGATKYHLNGRDSLVNPGPAMATHKDDEDKFFPMSGVLIFGKQGAGNSNVDLDNVMITSEE |
| | EIKTTNPVATEEYGTVATNLQSSNTAPATGTVNSQGALPGMVWQNRDVYLQGPIWAKIPHTDG |
| | HFHPSPLMGGFGLKHPPPQILIKNTPVPANPPTTFSPAKFASFITQYSTGQVSVEIEWELQKENSK |
| | RWNPEIQYTSNYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 147 | GEPVNAADAAALEHDKAYDQQLKAGDNPYLRYNHADAEFQERLQEDTSFGGNLGRAVFQAKK |
| (Anc80L4 | RVLEPLGLVEEGAKTAPGKKRPVEQSPQEPDSSSGIGKKGQQPAKKRLNFGQTGDSESVPDPQPL |
| 4) | GEPPAAPSGVGSNTMASGGGAPMADNNEGADGVGNASGNWHCDSTWLGDRVITTSTRTWA |
| | LPTYNNHLYKQISSQSGGSTNDNTYFGYSTPWGYFDFNRFHCHFSPRDWQRLINNNWGFRPKK |
| | LNFKLFNIQVKEVTTNDGTTTIANNLTSTVQVFTDSEYQLPYVLGSAHQGCLPPFPADVFMIPQY |
| | GYLTLNNGSQAVGRSSFYCLEYFPSQMLRTGNNFQFSYTFEDVPFHSSYAHSQSLDRLMNPLIDQ |
| | YLYYLSRTQTTSGTAGNRELQFSQAGPSSMANQAKNWLPGPCYRQQRVSKTTNQNNNSNFAW |
| | TGATKYHLNGRDSLVNPGPAMATHKDDEDKFFPMSGVLIFGKQGAGNSNVDLDNVMITNEEEI |
| | KTTNPVATEQYGTVATNLQSANTAPATGTVNSQGALPGMVWQDRDVYLQGPIWAKIPHTDG |
| | HFHPSPLMGGFGLKHPPPQILIKNTPVPANPPTTFSPAKFASFITQYSTGQVSVEIEWELQKENSK |
| | RWNPEIQYTSNYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 148 | GEPVNAADAAALEHDKAYDQQLKAGDNPYLRYNHADAEFQERLQEDTSFGGNLGRAVFQAKK |
| (Anc80L5 | RVLEPLGLVEEGAKTAPGKKRPVEQSPQEPDSSSGIGKKGQQPAKKRLNFGQTGDSESVPDPQPL |
| 9) | GEPPAAPSGVGSNTMASGGGAPMADNNEGADGVGNASGNWHCDSTWLGDRVITTSTRTWA |
| | LPTYNNHLYKQISSQSGASTNDNTYFGYSTPWGYFDFNRFHCHFSPRDWQRLINNNWGFRPKR |
| | LNFKLFNIQVKEVTTNDGTTTIANNLTSTVQVFTDSEYQLPYVLGSAHQGCLPPFPADVFMIPQY |
| | GYLTLNNGSQAVGRSSFYCLEYFPSQMLRTGNNFQFSYTFEDVPFHSSYAHSQSLDRLMNPLIDQ |
| | YLYYLSRTQTTSGTAGNRELQFSQAGPSSMANQAKNWLPGPCYRQQRVSKTTNQNNNSNFAW |
| | TGATKYHLNGRDSLVNPGPAMATHKDDEDKFFPMSGVLIFGKQGAGNSNVDLDNVMITNEEEI |
| | KTTNPVATEEYGTVATNLQSANTAPATGTVNSQGALPGMVWQNRDVYLQGPIWAKIPHTDGH |
| | FHPSPLMGGFGLKHPPPQILIKNTPVPANPPTTFSPAKFASFITQYSTGQVSVEIEWELQKENSKR |
| | WNPEIQYTSNYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 149 | GEPVNAADAAALEHDKAYDQQLKAGDNPYLRYNHADAEFQERLQEDTSFGGNLGRAVFQAKK |
| (Anc80L6 | RVLEPLGLVEEGAKTAPGKKRPVEQSPQEPDSSSGIGKKGQQPARKRLNFGQTGDSESVPDPQPL |
| 0) | GEPPAAPSGVGSNTMAAGGGAPMADNNEGADGVGNASGNWHCDSTWLGDRVITTSTRTW |
| | ALPTYNNHLYKQISSQSGGSTNDNTYFGYSTPWGYFDFNRFHCHFSPRDWQRLINNNWGFRPK |
| | RLNFKLFNIQVKEVTTNDGTTTIANNLTSTVQVFTDSEYQLPYVLGSAHQGCLPPFPADVFMIPQY |
| | GYLTLNNGSQAVGRSSFYCLEYFPSQMLRTGNNFEFSYTFEDVPFHSSYAHSQSLDRLMNPLIDQ |
| | YLYYLSRTQTTSGTAGNRELQFSQAGPSSMANQAKNWLPGPCYRQQRVSKTTNQNNNSNFAW |
| | TGATKYHLNGRDSLVNPGPAMATHKDDEDKFFPMSGVLIFGKQGAGNSNVDLDNVMITSEEEI |
| | KTTNPVATEEYGTVATNLQSSNTAPATGTVNSQGALPGMVWQERDVYLQGPIWAKIPHTDGH |
| | FHPSPLMGGFGLKHPPPQILIKNTPVPANPPTTFSPAKFASFITQYSTGQVSVEIEWELQKENSKR |
| | WNPEIQYTSNYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 150 | GEPVNAADAAALEHDKAYDQQLKAGDNPYLRYNHADAEFQERLQEDTSFGGNLGRAVFQAKK |
| (Anc80L6 | RVLEPLGLVEEGAKTAPGKKRPVEQSPQEPDSSSGIGKKGQQPARKRLNFGQTGDSESVPDPQPL |
| 2) | GEPPAAPSGVGSNTMASGGGAPMADNNEGADGVGNASGNWHCDSTWLGDRVITTSTRTWA |
| | LPTYNNHLYKQISSQSGGSTNDNTYFGYSTPWGYFDFNRFHCHFSPRDWQRLINNNWGFRPKK |
| | LNFKLFNIQVKEVTTNDGTTTIANNLTSTVQVFTDSEYQLPYVLGSAHQGCLPPFPADVFMIPQY |
| | GYLTLNNGSQAVGRSSFYCLEYFPSQMLRTGNNFEFSYTFEDVPFHSSYAHSQSLDRLMNPLIDQ |
| | YLYYLSRTQTTSGTAGNRELQFSQAGPSSMANQAKNWLPGPCYRQQRVSKTTNQNNNSNFAW |
| | TGATKYHLNGRDSLVNPGPAMATHKDDEDKFFPMSGVLIFGKQGAGNSNVDLDNVMITSEEEI |
| | KTTNPVATEEYGTVATNLQSANTAPATGTVNSQGALPGMVWQDRDVYLQGPIWAKIPHTDGH |
| | FHPSPLMGGFGLKHPPPQILIKNTPVPANPPTTFSPAKFASFITQYSTGQVSVEIEWELQKENSKR |
| | WNPEIQYTSNYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFNGLDK |
| NO: 151 | GEPVNAADAAALEHDKAYDQQLKAGDNPYLRYNHADAEFQERLQEDTSFGGNLGRAVFQAKK |
| (Anc82DI) | RVLEPLGLVEEGAKTAPGKKRPVEQSPQREPDSSTGIGKSGQQPAKKRLNFGQTGDSESVPDPQ |
| | PLGEPPAAPSGVGSNTMASGGGAPMADNNEGADGVGNSSGNWHCDSTWLGDRVITTSTRT |
| | WALPTYNNHLYKQISNGTSGGSTNDNTYFGYSTPWGYFDFNRFHCHFSPRDWQRLINNNWGF |
| | RPKRLNFKLFNIQVKEVTTNEGTKTIANNLTSTVQVFTDSEYQLPYVLGSAHQGCLPPFPADVFMI |
| | PQYGYLTLNNGSQAVGRSSFYCLEYFPSQMLRTGNNFQFSYTFEDVPFHSSYAHSQSLDRLMNP |
| | LIDQYLYYLSRTQTTGGTAGTQTLQFSQAGPSSMANQARNWVPGPCYRQQRVSTTTNQNNNS |
| | NFAWTGATKYHLNGRDSLMNPGVAMASHKDDEDRFFPSSGVLIFGKQGAGNDNVDYSNVMI |
| | TSEEEIKTTNPVATEEYGVVATNHQSANTQAQTGTVQNQGILPGMVWQNRDVYLQGPIWAKIP |
| | HTDGNFHPSPLMGGFGLKHPPPQILIKNTPVPADPPTTFNQAKLNSFITQYSTGQVSVEIEWELQ |
| | KENSKRWNPEIQYTSNYYKSTNVDFAVNTEGVYSEPRPIGTRYLTRNL |
| SEQ ID | MAADGYLPDWLEDNLSEGIREWWDLKPGAPKPKANQQKQDNGRGLVLPGYKYLGPFNGLDK |
| NO: 152 | GEPVNAADAAALEHDKAYDQQLQAGDNPYLRYNHADAEFQERLQEDTSFGGNLGRAVFQAKK |
| (AAV rh. | RVLEPLGLVESPVKTAPGKKRPVEPSPQRSPDSSTGIGKKGQQPAKKRLNFGQTGDSESVPDPQP |
| 74) | IGEPPAGPSGLGSGTMAAGGGAPMADNNEGADGVGSSSGNWHCDSTWLGDRVITTSTRTWA |
| | LPTYNNHLYKQISNGTSGGSTNDNTYFGYSTPWGYFDFNRFHCHFSPRDWQRLINNNWGFRPK |
| | RLNFKLFNIQVKEVTQNEGTKTIANNLTSTIQVFTDSEYQLPYVLGSAHQGCLPPFPADVFMIPQY |
| | GYLTLNNGSQAVGRSSFYCLEYFPSQMLRTGNNFEFSYNFEDVPFHSSYAHSQSLDRLMNPLIDQ |
| | YLYYLSRTQSTGGTAGTQQLLFSQAGPNNMSAQAKNWLPGPCYRQQRVSTTLSQNNNSNFAW |
| | TGATKYHLNGRDSLVNPGVAMATHKDDEERFFPSSGVLMFGKQGAGKDNVDYSSVMLTSEEEI |
| | KTTNPVATEQYGVVADNLQQQNAAPIVGAVNSQGALPGMVWQNRDVYLQGPIWAKIPHTDG |
| | NFHPSPLMGGFGLKHPPPQILIKNTPVPADPPTTFNQAKLASFITQYSTGQVSVEIEWELQKENSK |
| | RWNPEIQYTSNYYKSTNVDFAVNTEGTYSEPRPIGTRYLTRNL |

What is claimed is:

1. In a method of treating a patient by non-systemic administration of a recombinant adeno-associated virus (rAAV) to a target site, the improvement comprising:
adjunctively administering to the patient via systemic administration a composition comprising at least one antibody that is capable of neutralizing the non-systemically administered rAAV.

2. The method of claim 1, wherein the at least one antibody binds to at least one capsid protein of the rAAV.

3. The method of any one of claims 1-2, wherein the rAAV has a capsid protein of an AAV selected from: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

4. The method of claim 3, wherein the at least one antibody is an IgG antibody.

5. The method of any one of claims 1-4, wherein the composition is capable of neutralizing an rAAV selected from AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

6. The method of any one of claims 1-5, wherein the composition comprises a monoclonal antibody.

7. The method of any one of claims 1-6, wherein the composition comprises polyclonal antibodies comprising a mixture of monoclonal antibodies.

8. The method of any one of claims 1-5, wherein the composition comprises polyclonal antibodies obtained from at least 100 donors.

9. The method of claim 6, wherein the composition comprises polyclonal antibodies obtained from at least 500, at least 1000, at least 5000, or at least 10,000 donors.

10. The method of any one of claims 1-9, wherein the composition is intravenous immunoglobulin (IVIG).

11. The method of claim 10, wherein the IVIG is a pool of antibodies obtained from: Gammagard™ Liquid® (Baxter HealthCare Corp), Gammagard™ S/D, Gammaplex™, Bivigam™, Carimune™ NF, Gamunex-C, Gammaked™, Flebogamma™ DIF, Octagam™, and Privigen™.

12. The method of any one of claims 1-11, wherein the composition comprises recombinant polyclonal antibodies.

13. The method of any one of claims 1-11, wherein the composition comprises recombinant IVIG (rIVIG).

14. The method of any one of claim 1-3 or 5, wherein the at least one antibody is an antigen-binding fragment selected from: a single chain Fv (scFv), a Fab, a (Fab′)₂, and an (ScFv)₂.

15. The method of any one of claim 1-3 or 5, wherein the composition comprises: a bispecific antibody, a single-domain antibody (dAb), a diabody (db), a nanobody, and a unibody.

16. The method of any one of claims 1-9, wherein the composition is administered by intravenous injection or infusion.

17. The method of any one of claims 1-16, wherein the composition is IVIG and the IVIG is administered at a dose ranging from 1-2000 mg/kg patient weight.

18. The method of claim 17, wherein the IVIG is administered at a dose ranging from 1-2 mg/kg patient weight.

19. The method of claim 17, wherein the IVIG is administered at a dose ranging from 1-10 mg/ml.

20. The method of claim 17, wherein the IVIG is administered at a dose ranging from 1-4 mg/ml.

21. The method of claim 17, wherein IVIG is administered as a single-dose.

22. The method of any one of claims 17-21, wherein the IVIG is administered daily.

23. The method of any one of claims 17-22, wherein the IVIG is administered 2 times a day, three times a day, or four times a day.

24. The method of any one of claims 17-23, wherein the IVIG is administered 2 times a day, three times a day, or four times a day.

25. The method of any one of claims 19-24, wherein the IVIG is administered for no more than one week.

26. The method of any one of claims 17-25, wherein the IVIG is administered for at least 5 minutes, at least 10 minutes, at least 15 minutes, at least 20 minutes, at least 25 minutes, or at least 30 minutes.

27. The method of any one of claims 1-26, wherein the composition is administered before administration of the rAAV to the patient.

28. The method of claim 27, wherein the composition is administered at least 30 minutes before, at least 1 hour before, at least 2 hours before, at least 4 hours before at least 6 hours before, or at least 24 hours before, administration of the rAAV to the patient.

29. The method of claim 27, wherein the composition is administered no more than one week before administration of the rAAV to the patient.

30. The method of any one of claims 1-30, wherein the composition is administered after administration of the rAAV to the patient.

31. The method of claim 30, wherein the composition is administered no more than 30 minutes after, at least 1 hour after, at least 2 hours after, at least 4 hours after, at least 6 hours after, at least 24 hours after, or no more than one week after administration of the rAAV to the patient.

32. The method of any one of claims 1-3128, wherein the composition is administered on the same day as administration of the rAAV to the patient.

33. The method of any one of claims 1-32, wherein the composition is administered at a sufficient amount to alter biodistribution of the rAAV following administration to the patient.

34. The method of claim 33, wherein altering rAAV biodistribution reduces infection of liver cells.

35. The method of claim 33, wherein altering rAAV biodistribution reduces infection of cells outside of the target site.

36. The method of any one of claims 1-35, wherein the composition is administered at a sufficient amount to reduce liver toxicity of the rAAV following administration to the patient.

37. The method of any one of claims 1-35, wherein the composition is administered at a sufficient amount to reduce toxicity of the rAAV outside of the target site following administration to the patient.

38. The method of any one of claims 1-37, wherein the composition is administered at a sufficient amount to neutralize rAAV released from the target site.

39. The method of any one of claim 1-38, wherein the target site is selected from: brain, spinal cord, muscle, eye, and ear of the patient.

40. The method of any one of claims 1-39, wherein the target site is not within the central nervous system of the patient.

41. A method of treating a patient comprising the steps of:
non-systemically administering to a target site of the patient a first pharmaceutical composition comprising a recombinant adeno associated virus (rAAV); and
adjunctively administering to the patient via systemic administration a second pharmaceutical composition comprising at least one antibody that is capable of neutralizing the non-systemically administered rAAV.

42. The method of claim 41, wherein the at least one antibody binds to a capsid protein of the rAAV.

43. The method of any one of claims 41-42, wherein the rAAV has a capsid protein of an AAV selected from: AAV2; AAV1; AAV6; AAV3; AAV LK03; AAV7; AAV8; AAV hu.37; AAV rh. 10; AAV9; AAV hu. 68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; Anc80DI; and a variant thereof.

44. The method of claim 43, wherein the AAV is AAV8 or AAV9.

45. The method of claim 43, wherein the AAV is rh.10.

46. The method of claim 43, wherein the AAV is hu.68.

47. The method of claim 43, wherein the AAV is Anc80L65.

48. The method of any one of claims 41-47, wherein the antibody is an IgG antibody.

49. The method of any one of claims 41-48, wherein the second pharmaceutical composition is capable of neutralizing an AAV selected from AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

50. The method of any one of claims 40-49, wherein the at least one antibody is a monoclonal antibody.

51. The method of any one of claims 40-50, wherein the second pharmaceutical composition comprises polyclonal antibodies comprising a mixture of monoclonal antibodies.

52. The method of any one of claims 41-51, wherein the second pharmaceutical composition comprises polyclonal antibodies obtained from at least 100 donors.

53. The method of claim 52, wherein the second pharmaceutical composition comprises polyclonal antibodies obtained from at least 500, 1000, 5000, or 10,000 donors.

54. The method of any one of claims 41-53, wherein the second pharmaceutical composition comprises intravenous immunoglobulin (IVIG).

55. The method of claim 55, wherein the IVIG is a pool of antibodies obtained from: Gammagard™ Liquid® (Baxter HealthCare Corp), Gammagard™ S/D, Gammaplex™, Bivigam™, Carimune™ NF, Gamunex-C, Gammaked™, Flebogamma™ DIF, Octagam™, and Privigen™.

56. The method of any one of claims 41-55, wherein the second pharmaceutical composition comprises recombinant polyclonal antibodies.

57. The method of any one of claims 41-55, wherein the second pharmaceutical composition comprises recombinant IVIG (rIVIG).

58. The method of any one of claims 41-57, wherein the at least one antibody comprises an antigen-binding fragment selected from: a single chain Fv (scFv), a Fab, a (Fab′)₂, and an (ScFv)₂.

59. The method of any one of claims 41-58, wherein the at least one antibody comprises: a bispecific antibody, a single-domain antibody (dAb), a diabody (db), a nanobody, a unibody, and a diabody.

60. The method of any one of claims 41-59, wherein the first pharmaceutical composition is administered locally.

61. The method of claim 60, wherein the target site is within the central nervous system.

62. The method of claim 61, wherein the target site is selected from: brain, spinal cord, muscle, eye, and ear of the patient.

63. The method of any one of claim 61, wherein the first pharmaceutical composition is administered by: intrathecal injection or infusion, intraocular injection, intravitreal injection or infusion, inner ear injection or infusion, intracerebroventricular injection or infusion, intracisternal magna injection (ICM) or infusion, subretinal injection or infusion, or intramuscular injection or infusion.

64. The method of claim 63, wherein intrathecal injection or infusion is lumbar injection or infusion.

65. The method of claim 61, wherein the first pharmaceutical composition is administered to the brain or spinal cord by intracerebral, intrathecal, intracranial, intracerebroventricular, or cisterna magna administration.

66. The method of any one of claims 41-65, wherein the second pharmaceutical composition is administered systemically.

67. The method of claim 66, wherein the second pharmaceutical composition is administered by intravenous injection or infusion.

68. The method of any one of claims 41-67, wherein the second pharmaceutical composition comprises IVIG and the IVIG is administered at a dose ranging from 1-2000 mg/kg.

69. The method of claim 68, wherein the IVIG is administered at a dose ranging from 1-2 mg/kg.

70. The method of claim 68, wherein the IVIG is administered at a dose ranging from 1-10 mg/ml.

71. The method of claim 68, wherein the IVIG is administered at a dose ranging from 1-4 mg/ml.

72. The method of claim 68, wherein IVIG is administered as a single-dose.

73. The method of any one of claims 68-72, wherein the IVIG is administered daily.

74. The method of any one of claims 68-72, wherein the IVIG is administered at least 2 times a day.

75. The method of any one of claims 41-74, wherein the second pharmaceutical composition is administered before administration of the first pharmaceutical composition to the patient.

76. The method of claim 75, wherein the second pharmaceutical composition is administered at least 30 minutes before, at least 1 hour before, or at least 2 hours before administration of the first pharmaceutical composition to the patient.

77. The method of claim 75, wherein the second pharmaceutical composition is administered at least one day before, at least one week before, or at least two weeks before administration of the first pharmaceutical composition to the patient.

78. The method of any one of claims 41-74, wherein the second pharmaceutical composition is administered after administration of the first pharmaceutical composition to the patient.

79. The method of claim 78, wherein the second pharmaceutical composition is administered at least 30 minutes after, at least 1 hour after, or at least 2 hours after administration of the first pharmaceutical composition to the patient.

80. The method of claim 78, wherein the second pharmaceutical composition is administered at least one day after, at least two days after, or at least one week after administration of the first pharmaceutical composition to the patient.

81. The method of any one of claims 41-74, wherein the first pharmaceutical composition and the second pharmaceutical composition are administered on the same day.

82. The method of any one of claims 41-81, wherein the second pharmaceutical composition is administered at a sufficient amount to alter biodistribution of the rAAV following administration to the patient.

83. The method of claim 82, wherein altering AAV biodistribution reduces rAAV infection of liver cells.

84. The method of claim 82, wherein altering AAV biodistribution reduces rAAV infection of cells outside of the target site.

85. The method of any one of claims 41-84, wherein the second pharmaceutical composition is administered at a sufficient amount to reduce liver toxicity of the rAAV following administration to the patient.

86. The method of any one of claims 41-85, wherein the second pharmaceutical composition is administered at a sufficient amount to reduce toxicity of the rAAV outside of the target site following administration to the patient.

87. The method of any one of claim 83-85, wherein the target site is selected from: brain, spinal cord, muscle, eye, and ear of the patient.

88. The method of any one of claims 41-87, wherein the target site is not within the central nervous system.

89. A kit for a combination therapy comprising: a first pharmaceutical composition comprising a recombinant adeno associated virus (rAAV); and a second pharmaceutical composition comprising at least one antibody capable of neutralizing non-systemically administered rAAV of the first pharmaceutical composition.

90. The kit of claim 89, wherein the at least one antibody binds to a capsid protein of the rAAV.

91. The kit of any one of claims 89-90, wherein the capsid protein of an AAV is selected from AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

92. The kit of any one of claim 89, wherein the at least one antibody comprises IgG.

93. The kit of any one of claims 89-92, wherein the second pharmaceutical composition is capable of neutralizing an AAV selected from AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

94. The kit of any one of claims 89-93, wherein the at least one antibody is a monoclonal antibody.

95. The kit of any one of claims 89-94, wherein the second pharmaceutical composition comprises polyclonal antibodies obtained from at least 100 donors.

96. The kit of claim 95, wherein the second pharmaceutical composition comprises polyclonal antibodies obtained from at least 500, 1000, 5000, or 10,000 donors.

97. The kit of any one of claims 89-96, wherein the second pharmaceutical composition comprises intravenous immunoglobulin (IVIG).

98. The kit of any one of claims 89-96, wherein the second pharmaceutical composition comprises recombinant intravenous immunoglobulin (rIVIG).

99. The kit of claim 98, wherein the IVIG is a pool of antibodies obtained from: Gammagard™ Liquid® (Baxter HealthCare Corp), Gammagard™ S/D, Gammaplex™, Bivigam™, Carimune™ NF, Gamunex-C, Gammaked™, Flebogamma™ DIF, Octagam™, and Privigen™.

100. The kit of any one of claims 89-99, wherein the second pharmaceutical composition comprises recombinant polyclonal antibodies.

101. The kit of any one of claims 89-100, wherein the second pharmaceutical composition comprises an AAV-neutralizing antigen-binding fragment selected from: a single chain Fv (scFv), a Fab, a (Fab′)₂, and an (ScFv)₂.

102. The kit of any one of claims 89-101, wherein the second pharmaceutical composition comprises: a bispecific antibody, a single-domain antibody (dAb), a diabody (db), a nanobody, a unibody, and a diabody.
